(19) United States
(12) Patent Application Publication
(10) Pub. No.: US 2023/0348877 A1
(43) Pub. Date: Nov. 2, 2023

(54) BASE EDITING ENZYMES

(71) Applicant: METAGENOMI, INC., Emeryville, CA (US)

(72) Inventors: Jyun-Liang LIN, Emeryville, CA (US); Alan BROOKS, Emeryville, CA (US); Cristina BUTTERFIELD, Emeryville, CA (US); Christopher BROWN, Emeryville, CA (US); Cindy CASTELLE, Emeryville, CA (US); Brian C. Thomas, Emeryville, CA (US)

(21) Appl. No.: 18/180,633

(22) Filed: Mar. 8, 2023

Related U.S. Application Data
(63) Continuation of application No. PCT/US2021/049931, filed on Sep. 10, 2021.
(60) Provisional application No. 63/077,060, filed on Sep. 11, 2020.

(51) Int. Cl. C12N 9/22; C12N 15/11

(52) U.S. Cl. CPC C12N 9/22; C12N 15/11; C12N 2310/20

(57) ABSTRACT

The present disclosure provides for endonuclease enzymes having distinguishing domain features, as well as methods of using such enzymes or variants thereof.

### CROSS-REFERENCE

This application is a continuation application of International Application No. PCT/US2021/049931 filed Sep. 10, 2021, entitled “BASE EDITING ENZYMES”, which claims the benefit of U.S. Provisional Application No. 63/077,060, filed on Sep. 11, 2020, entitled “BASE EDITING ENZYMES”, each of which is incorporated in its entirety herein.

### BACKGROUND

Cas enzymes along with their associated Clustered Regularly Interspaced Short Palindromic Repeats (CRISPR) guide ribonucleic acids (RNAs) appear to be a pervasive (˜45% of bacteria, ˜84% of archaea) component of prokaryotic immune systems, serving to protect such microorganisms against non-self nucleic acids, such as infectious viruses and plasmids by CRISPR-RNA guided nucleic acid cleavage. While the deoxyribonucleic acid (DNA) elements encoding CRISPR RNA elements may be relatively conserved in structure and length, their CRISPR-associated (Cas) proteins are highly diverse, containing a wide variety of nucleic acid-interacting domains. While CRISPR DNA elements have been observed as early as 1987, the programmable endonuclease cleavage ability of CRISPR/Cas complexes has only been recognized relatively recently, leading to the use of recombinant CRISPR/Cas systems in diverse DNA manipulation and gene editing applications.

### SEQUENCE LISTING

The instant application contains a Sequence Listing which has been submitted electronically in XML format and is hereby incorporated by reference in its entirety. Said XML copy, created on Mar. 3, 2023, is named 55921-715303.xml and is 341,755 bytes in size.

### SUMMARY

In some aspects, the present disclosure provides for an engineered nucleic acid editing system, comprising: an endonuclease comprising a RuvC domain and an HNH domain, wherein said endonuclease is derived from an uncultivated microorganism, wherein said endonuclease is a class 2, type II Cas endonuclease, wherein said endonuclease is configured to be deficient in nuclease activity; a base editor coupled to said endonuclease; and an engineered guide ribonucleic acid structure configured to form a complex with said endonuclease comprising: a guide ribonucleic acid sequence configured to hybridize to a target deoxyribonucleic acid sequence; and a ribonucleic acid sequence configured to bind to said endonuclease. In some embodiments, said endonuclease comprises an aspartate to alanine mutation at residue 9 relative to SEQ ID NO: 23, residue 10 relative to SEQ ID NO: 22, or residue 28 relative to SEQ ID NO: 21 when optimally aligned. In some embodiments, said endonuclease comprises a sequence with at least 95% sequence identity to any one of SEQ ID NOs: 21-23 or a variant thereof. In some aspects, the present disclosure provides for an engineered nucleic acid editing system comprising: an endonuclease having at least 95% sequence identity to any one of SEQ ID NOs: 21-23 or a variant thereof, wherein said endonuclease comprises a RuvC domain lacking nuclease activity; a base editor coupled to said endonuclease; and an engineered guide ribonucleic acid structure configured to form a complex with said endonuclease comprising: a guide ribonucleic acid sequence configured to hybridize to a target deoxyribonucleic acid sequence; and a ribonucleic acid sequence configured to bind to said endonuclease. In some aspects, the present disclosure provides for an engineered nucleic acid editing system comprising: an endonuclease configured to bind to a protospacer adjacent motif (PAM) sequence comprising SEQ ID NOs: 145-147, wherein said endonuclease is a class 2, type II Cas endonuclease, and wherein said endonuclease is configured to be deficient in nuclease activity; and a base editor coupled to said endonuclease; and an engineered guide ribonucleic acid structure configured to form a complex with said endonuclease comprising: a guide ribonucleic acid sequence configured to hybridize to a target deoxyribonucleic acid sequence; and a ribonucleic acid sequence configured to bind to said endonuclease. In some embodiments, said endonuclease further comprises a RuvC and an HNH domain. In some embodiments, said ribonucleic acid sequence configured to bind to said endonuclease comprises a tracr sequence. In some embodiments, said endonuclease comprises a nickase mutation. In some embodiments, said RuvC domain lacks nuclease activity. In some embodiments, said endonuclease is configured to cleave one strand of a double-stranded target deoxyribonucleic acid. In some embodiments, said endonuclease has less than 80% identity to a Cas9 endonuclease. In some embodiments, said ribonucleic acid sequence comprises a sequence with at least 80% sequence identity to about 60 to 90 consecutive non-degenerate nucleotides selected from any one of SEQ ID NOs: 27-29, or a sequence with at least 80% sequence identity to non-degenerate nucleotides of any one of SEQ ID NOs: 27-29.

In some aspects, the present disclosure provides for an engineered nucleic acid editing system comprising, an engineered guide ribonucleic acid structure comprising: a guide ribonucleic acid sequence configured to hybridize to a target deoxyribonucleic acid sequence; and a ribonucleic acid sequence configured to bind to an endonuclease, wherein said ribonucleic acid sequence comprises a sequence with at least 80% sequence identity to about 60 to 90 consecutive non-degenerate nucleotides selected from any one of SEQ ID NOs: 27-29 or a sequence with at least 80% sequence identity to non-degenerate nucleotides of any one of SEQ ID NOs: 27-29; and a class 2, type II Cas endonuclease configured to bind to said engineered guide ribonucleic acid. In some embodiments, said endonuclease is configured to bind to a protospacer adjacent motif (PAM) sequence selected from the group consisting of SEQ ID NOs: 145-147. In some embodiments, said base editor comprises a sequence with at least 70%, 80%, 90% or 95% identity to any one of SEQ ID NOs: 1-17 or a variant thereof. In some embodiments, said base editor is an adenine deaminase. In some embodiments, said adenosine deaminase comprises a sequence with at least 95% identity to any one of SEQ ID NOs: 8 or 164 or a variant thereof. In some embodiments, said base editor is a cytosine deaminase. In some embodiments, said cytosine deaminase comprises a sequence with at least 95% identity to any one of SEQ ID NOs: 1-7, 9-17 or a variant thereof. In some embodiments, the engineered nucleic acid editing system further comprises a uracil DNA glycosylase inhibitor. In some embodiments, said uracil DNA glycosylase inhibitor comprises a sequence with at least 70%, 80%, 90% or 95% identity to SEQ ID NO: 18 or a variant thereof. In some embodiments, said engineered guide ribonucleic acid structure comprises at least two ribonucleic acid polynucleotides. In some embodiments, said engineered guide ribonucleic acid structure comprises one ribonucleic acid polynucleotide comprising said guide ribonucleic acid sequence and said tracr ribonucleic acid sequence. In some embodiments, said guide ribonucleic acid sequence is complementary to a prokaryotic, bacterial, archaeal, eukaryotic, fungal, plant, mammalian, or human genomic sequence. In some embodiments, said guide ribonucleic acid sequence is 15-24 nucleotides in length. In some embodiments, said endonuclease comprises one or more nuclear localization sequences (NLSs) proximal to an N- or C-terminus of said endonuclease. In some embodiments, said NLS comprises a sequence with at least 90% identity to a selected from SEQ ID NOs: 148-163 or a variant thereof. In some embodiments, said endonuclease is covalently coupled directly to said base editor or covalently coupled to said base editor through a linker. In some embodiments, a polypeptide comprises said endonuclease and said base editor. In some embodiments, said endonuclease is configured to cleave one strand of a double-stranded target deoxyribonucleic acid. In some embodiments, said system further comprises a source of Mg²⁺. In some embodiments: said endonuclease comprises a sequence having at least 70%, at least 80%, or at least 90% sequence identity to SEQ ID NO: 21 or a variant thereof; said guide RNA structure comprises a sequence having at least 70%, at least 80%, or at least 90% sequence identity to SEQ ID NO: 27; said endonuclease is configured to bind to a PAM comprising SEQ ID NO: 145; or said base editor comprises a sequence having at least 70%, at least 80%, or at least 90% sequence identity to SEQ ID NO: 164 or a variant thereof. In some embodiments: said endonuclease comprises a sequence at least 70%, at least 80%, or at least 90% identical to SEQ ID NO: 22 or a variant thereof, said guide RNA structure comprises a sequence having at least 70%, at least 80%, or at least 90% sequence identity to SEQ ID NO: 28; and said endonuclease is configured to bind to a PAM comprising SEQ ID NO: 146. In some embodiments: said endonuclease comprises a sequence at least 70%, at least 80%, or at least 90% identical to SEQ ID NO: 23 or a variant thereof; said guide RNA structure comprises a sequence having at least 70%, at least 80%, or at least 90% sequence identity to SEQ ID NO: 29; and said endonuclease is configured to bind to a PAM comprising SEQ ID NO: 147. In some embodiments, said base editor comprises an adenine deaminase. In some embodiments, said adenine deaminase comprises a sequence having at least 70%, at least 80%, or at least 90% sequence identity to any one of SEQ ID NOs: 8 or 164. In some embodiments, said base editor comprises a cytosine deaminase. In some embodiments, said cytosine deaminase comprises a sequence having at least 70%, at least 80%, or at least 90% sequence identity to any one of SEQ ID NOs: 1-7 or 9-17. In some embodiments, the engineered nucleic acid editing system further comprises a uracil DNA glycosylation inhibitor optionally coupled to said endonuclease or said base editor. In some embodiments, said uracil DNA glycosylation inhibitor comprises a sequence having at least 70%, at least 80%, or at least 90% sequence identity to SEQ ID NO: 18 or a variant thereof. In some embodiments, said sequence identity is determined by a BLASTP, CLUSTALW, MUSCLE, MAFFT, or Smith-Waterman homology search algorithm. In some embodiments, said sequence identity is determined by said BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some aspects, the present disclosure provides for a nucleic acid comprising an engineered nucleic acid sequence optimized for expression in an organism, wherein said nucleic acid encodes a class 2, type II Cas endonuclease coupled to a base editor, and wherein said endonuclease is derived from an uncultivated microorganism.

In some aspects, the present disclosure provides for a nucleic acid comprising an engineered nucleic acid sequence optimized for expression in an organism, wherein said nucleic acid encodes an endonuclease having at least 70% sequence identity to any one of SEQ ID NOs: 21-23 coupled to a base editor. In some embodiments, said endonuclease comprises a sequence encoding one or more nuclear localization sequences (NLSs) proximal to an N- or C-terminus of said endonuclease. In some embodiments, said NLS comprises a sequence with at least 90% identity to any one of SEQ ID NOs: 148-163 or a variant thereof. In some embodiments, said organism is prokaryotic, bacterial, eukaryotic, fungal, plant, mammalian, rodent, or human.

In some aspects, the present disclosure provides for a vector comprising a nucleic acid sequence encoding a class 2, type II Cas endonuclease coupled to a base editor, wherein said endonuclease is derived from an uncultivated microorganism. In some embodiments, the endonuclease comprises any of the nucleases of any of the aspects or embodiments described herein.

In some aspects, the present disclosure provides for a vector comprising the nucleic acid of any of the aspects or embodiments described herein. In some embodiments, the vector further comprises a nucleic acid encoding an engineered guide ribonucleic acid structure configured to form a complex with said endonuclease comprising: a guide ribonucleic acid sequence configured to hybridize to a target deoxyribonucleic acid sequence; and a tracr ribonucleic acid sequence configured to binding to said endonuclease. In some embodiments, the vector is a plasmid, a minicircle, a CELiD, an adeno-associated virus (AAV) derived virion, or a lentivirus.

In some aspects, the present disclosure provides for a cell comprising the vector of any of the aspects or embodiments described herein.

In some aspects, the present disclosure provides for a method of manufacturing an endonuclease, comprising cultivating said cell of any of the aspects or embodiments described herein.

In some aspects, the present disclosure provides for a method for modifying a double-stranded deoxyribonucleic acid polynucleotide comprising contacting said double-stranded deoxyribonucleic acid polynucleotide with a complex comprising: an endonuclease comprising a RuvC domain and an HNH domain, wherein said endonuclease is derived from an uncultivated microorganism, wherein said endonuclease is a class 2, type II Cas endonuclease, and said endonuclease is configured to be deficient in nuclease activity; a base editor coupled to said endonuclease; and an engineered guide ribonucleic acid structure configured to bind to said endonuclease and said double-stranded deoxyribonucleic acid polynucleotide; wherein said double-stranded deoxyribonucleic acid polynucleotide comprises a protospacer adjacent motif (PAM). In some embodiments, said endonuclease comprises a nickase mutation. In some embodiments, said RuvC domain lacks nuclease activity. In some embodiments, said endonuclease is configured to cleave one strand of a double-stranded target deoxyribonucleic acid. In some embodiments, said endonuclease comprising a RuvC domain and an HNH domain is covalently coupled directly to said base editor or covalently coupled to said base editor through a linker. In some embodiments, said endonuclease comprising a RuvC domain and an HNH domain comprises a sequence with at least 95% sequence identity to any one of SEQ ID NOs: 21-23 or a variant thereof.

In some aspects, the present disclosure provides for a method for modifying a double-stranded deoxyribonucleic acid polynucleotide, comprising contacting said double-stranded deoxyribonucleic acid polynucleotide with a complex comprising: a class 2, type II Cas endonuclease, a base editor coupled to said endonuclease, and an engineered guide ribonucleic acid structure configured to bind to said endonuclease and said double-stranded deoxyribonucleic acid polynucleotide; wherein said double-stranded deoxyribonucleic acid polynucleotide comprises a protospacer adjacent motif (PAM); and wherein said PAM comprises a sequence selected from the group consisting of SEQ ID NOs: 145-147. In some embodiments, said class 2, type II Cas endonuclease is covalently coupled to said base editor or coupled to said base editor through a linker. In some embodiments, said base editor comprises a sequence with at least 70%, at least 80%, at least 90% or at least 95% identity to a sequence selected from SEQ ID NOs: 1-17 or a variant thereof. In some embodiments, said base editor comprises an adenine deaminase; said double-stranded deoxyribonucleic acid polynucleotide comprises an adenine; and modifying said double-stranded deoxyribonucleic acid polypeptide comprises converting said adenine to guanine. In some embodiments, said adenine deaminase comprises a sequence with at least 70%, 80%, 90% or 95% sequence identity to any one of SEQ ID NOs: 8 or 164 or a variant thereof. In some embodiments, said base editor comprises a cytosine deaminase; said double-stranded deoxyribonucleic acid polynucleotide comprises a cytosine; and modifying said double-stranded deoxyribonucleic acid polypeptide comprises converting said cytosine to uracil. In some embodiments, said cytosine deaminase comprises a sequence with at least 70%, 80%, 90% or 95% sequence identity to any one of SEQ ID NOs: 1-7 or 9-17. In some embodiments, said complex further comprises a uracil DNA glycosylase inhibitor. In some embodiments, said uracil DNA glycosylase inhibitor comprises a sequence with at least 70%, 80%, 90% or 95% identity to SEQ ID NO: 18 or a variant thereof. In some embodiments, said double-stranded deoxyribonucleic acid polynucleotide comprises a first strand comprising a sequence complementary to a sequence of said engineered guide ribonucleic acid structure and a second strand comprising said PAM. In some embodiments, said PAM is directly adjacent to the 3′ end of said sequence complementary to said sequence of said engineered guide ribonucleic acid structure. In some embodiments, said class 2, type II Cas endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas 13d endonuclease. In some embodiments, said class 2, type II Cas endonuclease is derived from an uncultivated microorganism. In some embodiments, said double-stranded deoxyribonucleic acid polynucleotide is a eukaryotic, plant, fungal, mammalian, rodent, or human double-stranded deoxyribonucleic acid polynucleotide.

In some aspects, the present disclosure provides for a method of modifying a target nucleic acid locus, said method comprising delivering to said target nucleic acid locus said engineered nucleic acid editing system of any of the aspects or embodiments described herein, wherein said endonuclease is configured to form a complex with said engineered guide ribonucleic acid structure, and wherein said complex is configured such that upon binding of said complex to said target nucleic acid locus, said complex modifies a nucleotide of said target nucleic locus. In some embodiments, said engineered nucleic acid editing system comprises an adenine deaminase, said nucleotide is an adenine, and modifying said target nucleic acid locus comprises converting said adenine to a guanine. In some embodiments, said engineered nucleic acid editing system comprises a cytidine deaminase and a uracil DNA glycosylase inhibitor, said nucleotide is a cytosine and modifying said target nucleic acid locus comprises converting said adenine to a uracil. In some embodiments, said target nucleic acid locus comprises genomic DNA, viral DNA, or bacterial DNA. In some embodiments, said target nucleic acid locus is in vitro. In some embodiments, said target nucleic acid locus is within a cell. In some embodiments, said cell is a prokaryotic cell, a bacterial cell, a eukaryotic cell, a fungal cell, a plant cell, an animal cell, a mammalian cell, a rodent cell, a primate cell, or a human cell. In some embodiments, said cell is within an animal. In some embodiments, said cell is within a cochlea. In some embodiments, said cell is within an embryo. In some embodiments, said embryo is a two-cell embryo. In some embodiments, said embryo is a mouse embryo. In some embodiments, delivering said engineered nucleic acid editing system to said target nucleic acid locus comprises delivering the nucleic acid of any of the aspects or embodiments described herein or the vector of any of the aspects or embodiments described herein. In some embodiments, said engineered nucleic acid editing system to said target nucleic acid locus comprises delivering a nucleic acid comprising an open reading frame encoding said endonuclease. In some embodiments, said nucleic acid comprises a promoter to which said open reading frame encoding said endonuclease is operably linked. In some embodiments, delivering said engineered nucleic acid editing system to said target nucleic acid locus comprises delivering a capped mRNA containing said open reading frame encoding said endonuclease. In some embodiments, delivering said engineered nucleic acid editing system to said target nucleic acid locus comprises delivering a translated polypeptide. In some embodiments, delivering said engineered nucleic acid editing system to said target nucleic acid locus comprises delivering a deoxyribonucleic acid (DNA) encoding said engineered guide ribonucleic acid structure operably linked to a ribonucleic acid (RNA) pol III promoter.

In some aspects, the present disclosure provides for an engineered nucleic acid editing polypeptide, comprising: an endonuclease comprising a RuvC domain and an HNH domain, wherein said endonuclease is derived from an uncultivated microorganism, wherein said endonuclease is a class 2, type II Cas endonuclease, wherein said endonuclease is configured to be deficient in nuclease activity; and a base editor coupled to said endonuclease. In some embodiments, said endonuclease comprises a nickase mutation. In some embodiments, said RuvC domain lacks nuclease activity. In some embodiments, said endonuclease is configured to cleave one strand of a double-stranded target deoxyribonucleic acid. In some embodiments, said endonuclease comprises a sequence with at least 95% sequence identity to any one of SEQ ID NOs: 21-23 or a variant thereof.

In some aspects, the present disclosure provides for an engineered nucleic acid editing polypeptide, comprising: an endonuclease having at least 95% sequence identity to any one of SEQ ID NOs: 21-23 or a variant thereof, wherein said endonuclease is configured to be deficient in nuclease activity; and a base editor coupled to said endonuclease. In some aspects, the present disclosure provides for an engineered nucleic acid editing polypeptide, comprising: an endonuclease configured to bind to a protospacer adjacent motif (PAM) sequence comprising SEQ ID NOs: 145-147, wherein said endonuclease is a class 2, type II Cas endonuclease, wherein said endonuclease is configured to be deficient in nuclease activity; and a base editor coupled to said endonuclease. In some embodiments, said endonuclease comprises a nickase mutation. In some embodiments, said endonuclease comprises a RuvC domain lacking nuclease activity. In some embodiments, said endonuclease is configured to cleave one strand of a double-stranded target deoxyribonucleic acid. In some embodiments, said endonuclease is derived from an uncultivated microorganism. In some embodiments, said endonuclease has less than 80% identity to a Cas9 endonuclease. In some embodiments, said endonuclease further comprises a RuvC and an HNH domain. In some embodiments, said tracr ribonucleic acid sequence comprises a sequence with at least 80% sequence identity to about 60 to 90 consecutive non-degenerate nucleotides selected from any one of SEQ ID NOs: 27-29, or a sequence with at least 80% sequence identity to non-degenerate nucleotides of any one of SEQ ID NOs: 27-29. In some embodiments, said base editor comprises a sequence with at least 70%, 80%, 90% or 95% sequence identity to any one of SEQ ID NOs: 1-17 or a variant thereof. In some embodiments, said base editor is an adenine deaminase. In some embodiments, said adenosine deaminase comprises a sequence with at least 70%, 80%, 90% or 95% sequence identity to any one of SEQ ID NOs: 8 or 164. In some embodiments, said base editor is a cytosine deaminase. In some embodiments, said cytosine deaminase comprises a sequence with at least 70%, 80%, 90% or 95% sequence identity to any one of SEQ ID NOs: 1-7 or 9-17.

In some aspects, the present disclosure provides for an engineered nucleic acid editing polypeptide, comprising: an endonuclease, wherein said endonuclease is configured to be deficient in endonuclease activity; and a base editor coupled to said endonuclease, wherein said base editor comprises a sequence with at least 70%, 80%, 90% or 95% sequence identity to any one of SEQ ID NOs: 1-7 or a variant thereof. In some embodiments, said endonuclease is configured to cleave one strand of a double-stranded target deoxyribonucleic acid. In some embodiments, said endonuclease is configured to be catalytically dead. In some embodiments, said endonuclease is a Cas endonuclease. In some embodiments, said Cas endonuclease is a Class II, type II Cas endonuclease or a Class II, type V Cas endonuclease. In some embodiments, said endonuclease comprises a sequence having at least 70%, 80%, 90% or 95% sequence identity to any one of SEQ ID NOs: 21-23 or a variant thereof. In some embodiments, said endonuclease comprises a nickase mutation. In some embodiments, said endonuclease comprises an aspartate to alanine mutation at residue 9 relative to SEQ ID NO: 23, residue 10 relative to SEQ ID NO: 22, or residue 28 relative to SEQ ID NO: 21 when optimally aligned. In some embodiments, said endonuclease is configured to bind to a protospacer adjacent motif (PAM) sequence selected from the group consisting of SEQ ID NOs: 145-147. In some embodiments, said base editor is an adenine deaminase. In some embodiments, said adenosine deaminase comprises a sequence with at least 70%, 80%, 90% or 95% identity to any one SEQ ID NOs: 8 or 164 or a variant thereof. In some embodiments, said base editor is a cytosine deaminase. In some embodiments, said cytosine deaminase comprises a sequence with at least 70%, 80%, 90% or 95% identity to any one of SEQ ID NOs: 8 or 164. In some embodiments, said engineered nucleic acid editing system further comprises a uracil DNA glycosylase inhibitor coupled to said endonuclease or said base editor. In some embodiments, said uracil DNA glycosylase inhibitor comprises a sequence with at least 70%, 80%, 90% or 95% identity to SEQ ID NO: 18 or a variant thereof. In some embodiments, said endonuclease comprises one or more nuclear localization sequences (NLSs) proximal to an N- or C-terminus of said endonuclease. In some embodiments, said NLS comprises a sequence with at least 90% identity to any one of SEQ ID NOs: 148-163 or a variant thereof. In some embodiments, said endonuclease is covalently coupled directly to said base editor or covalently coupled to said base editor through a linker.

In some aspects, the present disclosure provides for a nucleic acid comprising an engineered nucleic acid sequence optimized for expression in an organism, wherein said nucleic acid encodes a sequence having at least 70%, 80%, 90% or 95% sequence identity to any one of SEQ ID NOs: 1-7 or a variant thereof. In some embodiments, said organism is prokaryotic, bacterial, eukaryotic, fungal, plant, mammalian, rodent, or human.

In some aspects, the present disclosure provides for a system comprising: (a) the nucleic acid editing polypeptide of any of the aspects or embodiments described herein; and (b) an engineered guide ribonucleic acid structure configured to form a complex with said nucleic acid editing polypeptide comprising: a guide ribonucleic acid sequence configured to hybridize to a target deoxyribonucleic acid sequence; and a ribonucleic acid sequence configured to bind to said endonuclease. In some embodiments, said engineered guide ribonucleic acid sequence comprises a sequence with at least 80% sequence identity to non-degenerate nucleotides of any one of SEQ ID NOs: 27-29.

In some aspects, the present disclosure provides for a method of modifying a target nucleic acid locus, said method comprising delivering to said target nucleic acid locus said engineered nucleic acid editing polypeptide of any of the aspects or embodiments described herein, or said system of any of the aspects or embodiments described herein, wherein said complex is configured such that upon binding of said complex to said target nucleic acid locus, said complex modifies a nucleotide of said target nucleic locus.

In some aspects, the present disclosure provides for an engineered nucleic acid editing system, comprising: (a) an endonuclease comprising a RuvC domain and an HNH domain, wherein the endonuclease is derived from an uncultivated microorganism, wherein the endonuclease is a class 2, type II Cas endonuclease, and wherein the RuvC domain lacks nuclease activity; (b) a base editor coupled to the endonuclease; and (c) an engineered guide ribonucleic acid structure configured to form a complex with the endonuclease comprising: (i) a guide ribonucleic acid sequence configured to hybridize to a target deoxyribonucleic acid sequence; and (ii) a tracr ribonucleic acid sequence configured to bind to the endonuclease. In some embodiments, the endonuclease comprises a sequence with at least 95% sequence identity to any one of SEQ ID NOs: 21-23.

In some aspects, the present disclosure provides for an engineered nucleic acid editing system comprising: (a) an endonuclease having at least 95% sequence identity to any one of SEQ ID NOs: 21-23, wherein the endonuclease comprises a RuvC domain lacking nuclease activity; a base editor coupled to the endonuclease; and an engineered guide ribonucleic acid structure configured to form a complex with the endonuclease comprising: (i) a guide ribonucleic acid sequence configured to hybridize to a target deoxyribonucleic acid sequence; and (ii) a tracr ribonucleic acid sequence configured to bind to the endonuclease.

In some aspects, the present disclosure provides for an engineered nucleic acid editing system comprising: (a) an endonuclease configured to bind to a protospacer adjacent motif (PAM) sequence comprising SEQ ID NOs: 145-147, wherein the endonuclease is a class 2, type II Cas endonuclease, and wherein the endonuclease comprises a RuvC domain lacking nuclease activity; and (b) a base editor coupled to the endonuclease; and (c) an engineered guide ribonucleic acid structure configured to form a complex with the endonuclease comprising: (i) a guide ribonucleic acid sequence configured to hybridize to a target deoxyribonucleic acid sequence; and (ii) a tracr ribonucleic acid sequence configured to bind to the endonuclease.

In some embodiments, the endonuclease is derived from an uncultivated microorganism. In some embodiments, the endonuclease has less than 80% identity to a Cas9 endonuclease. In some embodiments, the endonuclease further comprises an HNH domain. In some embodiments, the tracr ribonucleic acid sequence comprises a sequence with at least 80% sequence identity to about 60 to 90 consecutive nucleotides selected from any one of SEQ ID NOs: 27-29.

In some aspects, the present disclosure provides an engineered nucleic acid editing system comprising, (a) an engineered guide ribonucleic acid structure comprising: (i) a guide ribonucleic acid sequence configured to hybridize to a target deoxyribonucleic acid sequence; and (ii) a tracr ribonucleic acid sequence configured to bind to an endonuclease, wherein the tracr ribonucleic acid sequence comprises a sequence with at least 80% sequence identity to about 60 to 90 consecutive nucleotides selected from any one of SEQ ID NOs: 27-29; and a class 2, type II Cas endonuclease configured to bind to the engineered guide ribonucleic acid.

In some embodiments, the endonuclease is configured to bind to a protospacer adjacent motif (PAM) sequence selected from the group consisting of SEQ ID NOs: 145-147. In some embodiments, the base editor comprises a sequence with at least 70%, 80%, 90% or 95% identity to any one of SEQ ID NOs: 1-7. In some embodiments, the base editor is an adenine deaminase. In some embodiments, the adenosine deaminase comprises a sequence with at least 95% identity to SEQ ID NO: 8. In some embodiments, the base editor is a cytosine deaminase. In some embodiments, the cytosine deaminase comprises a sequence with at least 95% identity to SEQ ID NO: 9. In some embodiments, the cytosine deaminase comprises a sequence with at least 95% identity to any one of SEQ ID NOs: 10-17.

In some embodiments, the engineered nucleic acid editing system further comprises a uracil DNA glycosylase inhibitor. In some embodiments, the uracil DNA glycosylase inhibitor comprises a sequence with at least 70%, 80%, 90% or 95% identity to SEQ ID NO: 18.

In some embodiments, the engineered guide ribonucleic acid structure comprises at least two ribonucleic acid polynucleotides. In some embodiments, the engineered guide ribonucleic acid structure comprises one ribonucleic acid polynucleotide comprising the guide ribonucleic acid sequence and the tracr ribonucleic acid sequence. In some embodiments, the guide ribonucleic acid sequence is complementary to a prokaryotic, bacterial, archaeal, eukaryotic, fungal, plant, mammalian, or human genomic sequence. In some embodiments, the guide ribonucleic acid sequence is 15-24 nucleotides in length. In some embodiments, the endonuclease comprises one or more nuclear localization sequences (NLSs) proximal to an N- or C-terminus of said endonuclease. In some embodiments, the endonuclease is covalently coupled directly to the base editor or covalently coupled to the base editor through a linker. In some embodiments, a polypeptide comprises the endonuclease and the base editor. In some embodiments, the endonuclease is configured to cleave one strand of a double-stranded target deoxyribonucleic acid. In some embodiments, the system further comprises a source of Mg²⁺.

In some embodiments, the endonuclease comprises a sequence at least 70%, at least 80%, or at least 90% identical to SEQ ID NO: 21; the guide RNA structure comprises a sequence at least 70%, at least 80%, or at least 90% identical to SEQ ID NO: 27; and the endonuclease is configured to bind to a PAM comprising SEQ ID NO: 145.

In some embodiments, the endonuclease comprises a sequence at least 70%, at least 80%, or at least 90% identical to SEQ ID NO: 22; the guide RNA structure comprises a sequence at least 70%, at least 80%, or at least 90% identical to SEQ ID NO: 28; and the endonuclease is configured to bind to a PAM comprising SEQ ID NO: 146.

In some embodiments, the endonuclease comprises a sequence at least 70%, at least 80%, or at least 90% identical to SEQ ID NO: 23; the guide RNA structure comprises a sequence at least 70%, at least 80%, or at least 90% identical to SEQ ID NO: 29; and the endonuclease is configured to bind to a PAM comprising SEQ ID NO: 147.

In some embodiments, the base editor comprises an adenine deaminase. In some embodiments, the adenine deaminase comprises SEQ ID NO: 8. In some embodiments, the base editor comprises a cytosine deaminase. In some embodiments, the cytosine deaminase comprises SEQ ID NO: 9. In some embodiments, the engineered nucleic acid editing system described herein further comprises a uracil DNA glycosylation inhibitor. In some embodiments, the uracil DNA glycosylation inhibitor comprises SEQ ID NO: 18.

In some embodiments, the sequence identity is determined by a BLASTP, CLUSTALW, MUSCLE, MAFFT, or Smith-Waterman homology search algorithm. In some embodiments, the sequence identity is determined by said BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some aspects, the present disclosure provides a nucleic acid comprising an engineered nucleic acid sequence optimized for expression in an organism, wherein the nucleic acid encodes a class 2, type II Cas endonuclease coupled to a base editor, and wherein the endonuclease is derived from an uncultivated microorganism.

In some aspects, the present disclosure provides a nucleic acid comprising an engineered nucleic acid sequence optimized for expression in an organism, wherein the nucleic acid encodes an endonuclease having at least 70% sequence identity to any one of SEQ ID NOs: 21-23 coupled to a base editor. In some embodiments, the endonuclease comprises a sequence encoding one or more nuclear localization sequences (NLSs) proximal to an N- or C-terminus of said endonuclease. In some embodiments, the NLS comprises a sequence with at least 90% identity to a selected from SEQ ID NOs: 148-163. In some embodiments, the organism is prokaryotic, bacterial, eukaryotic, fungal, plant, mammalian, rodent, or human.

In some aspects, the present disclosure provides a vector comprising a nucleic acid sequence encoding a class 2, type II Cas endonuclease coupled to a base editor, wherein said endonuclease is derived from an uncultivated microorganism. In some embodiments, the vector comprises the nucleic acid described herein. In some embodiments, the vector further comprises a nucleic acid encoding an engineered guide ribonucleic acid structure configured to form a complex with the endonuclease comprising: a guide ribonucleic acid sequence configured to hybridize to a target deoxyribonucleic acid sequence; and a tracr ribonucleic acid sequence configured to binding to the endonuclease. In some embodiments, the vector is a plasmid, a minicircle, a CELiD, an adeno-associated virus (AAV) derived virion, or a lentivirus. In some aspects, the present disclosure provides a cell comprising the vector described herein. In some aspects, the present disclosure provides a method of manufacturing an endonuclease, comprising cultivating the cell described herein.

In some aspects, the present disclosure provides a method for modifying a double-stranded deoxyribonucleic acid polynucleotide comprising contacting the double-stranded deoxyribonucleic acid polynucleotide with a complex comprising: an endonuclease comprising a RuvC domain and an HNH domain, wherein the endonuclease is derived from an uncultivated microorganism, wherein the endonuclease is a class 2, type II Cas endonuclease, and wherein the RuvC domain lacks nuclease activity; a base editor coupled to the endonuclease; and an engineered guide ribonucleic acid structure configured to bind to the endonuclease and the double-stranded deoxyribonucleic acid polynucleotide; wherein the double-stranded deoxyribonucleic acid polynucleotide comprises a protospacer adjacent motif (PAM).

In some embodiments, the endonuclease comprising a RuvC domain and an HNH domain is covalently coupled directly to the base editor or covalently coupled to the base editor through a linker. In some embodiments, the endonuclease comprising a RuvC domain and an HNH domain comprises a sequence with at least 95% sequence identity to any one of SEQ ID NOs: 21-23.

In some aspects, the present disclosure provides a method for modifying a double-stranded deoxyribonucleic acid polynucleotide, comprising contacting the double-stranded deoxyribonucleic acid polynucleotide with a complex comprising: a class 2, type II Cas endonuclease, a base editor coupled to the endonuclease, and an engineered guide ribonucleic acid structure configured to bind to the endonuclease and the double-stranded deoxyribonucleic acid polynucleotide; wherein the double-stranded deoxyribonucleic acid polynucleotide comprises a protospacer adjacent motif (PAM); and wherein the PAM comprises a sequence selected from the group consisting of SEQ ID NOs: 145-147.

In some embodiments, the class 2, type II Cas endonuclease is covalently coupled to the base editor or coupled to the base editor through a linker. In some embodiments, the base editor comprises a sequence with at least 70%, at least 80%, at least 90% or at least 95% identity to a sequence selected from SEQ ID NOs: 1-7. In some embodiments, the base editor comprises an adenine deaminase; the double-stranded deoxyribonucleic acid polynucleotide comprises an adenine; and modifying the double-stranded deoxyribonucleic acid polypeptide comprises converting the adenine to guanine. In some embodiments, the adenine deaminase comprises a sequence with at least 95% identity to SEQ ID NO: 8.

In some embodiments, the base editor comprises a cytosine deaminase; the double-stranded deoxyribonucleic acid polynucleotide comprises a cytosine; and modifying the double-stranded deoxyribonucleic acid polypeptide comprises converting the cytosine to uracil. In some embodiments, the cytosine deaminase comprises a sequence with at least 95% identity to SEQ ID NO: 9. In some embodiments, the cytosine deaminase comprises a sequence with at least 95% identity to any one of SEQ ID NOs: 10-17.

In some embodiments, the complex further comprises a uracil DNA glycosylase inhibitor. In some embodiments, the uracil DNA glycosylase inhibitor comprises a sequence with at least 70%, 80%, 90% or 95% identity to SEQ ID NO: 18. In some embodiments, the double-stranded deoxyribonucleic acid polynucleotide comprises a first strand comprising a sequence complementary to a sequence of the engineered guide ribonucleic acid structure and a second strand comprising said PAM. In some embodiments, the PAM is directly adjacent to the 3′ end of the sequence complementary to the sequence of the engineered guide ribonucleic acid structure.

In some embodiments, the class 2, type II Cas endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas 13d endonuclease. In some embodiments, the class 2, type II Cas endonuclease is derived from an uncultivated microorganism. In some embodiments, the double-stranded deoxyribonucleic acid polynucleotide is a eukaryotic, plant, fungal, mammalian, rodent, or human double-stranded deoxyribonucleic acid polynucleotide.

In some aspects, the present disclosure provides a method of modifying a target nucleic acid locus, said method comprising delivering to said target nucleic acid locus the engineered nucleic acid editing system described herein, wherein the endonuclease is configured to form a complex with the engineered guide ribonucleic acid structure, and wherein the complex is configured such that upon binding of the complex to the target nucleic acid locus, the complex modifies a nucleotide of the target nucleic locus.

In some embodiments, the engineered nucleic acid editing system comprises an adenine deaminase, the nucleotide is an adenine, and modifying the target nucleic acid locus comprises converting the adenine to a guanine. In some embodiments, the engineered nucleic acid editing system comprises a cytidine deaminase and a uracil DNA glycosylase inhibitor, the nucleotide is a cytosine and modifying the target nucleic acid locus comprises converting the adenine to a uracil. In some embodiments, the target nucleic acid locus comprises genomic DNA, viral DNA, or bacterial DNA. In some embodiments, the target nucleic acid locus is in vitro. In some embodiments, the target nucleic acid locus is within a cell. In some embodiments, the cell is a prokaryotic cell, a bacterial cell, a eukaryotic cell, a fungal cell, a plant cell, an animal cell, a mammalian cell, a rodent cell, a primate cell, or a human cell. In some embodiments, the cell is within an animal.

In some embodiments, the cell is within a cochlea. In some embodiments, the cell is within an embryo. In some embodiments, the embryo is a two-cell embryo. In some embodiments, the embryo is a mouse embryo. In some embodiments, delivering the engineered nucleic acid editing system to the target nucleic acid locus comprises delivering the nucleic acid described herein or the vector described herein. In some embodiments, delivering the engineered nucleic acid editing system to the target nucleic acid locus comprises delivering a nucleic acid comprising an open reading frame encoding the endonuclease.

In some embodiments, the nucleic acid comprises a promoter to which the open reading frame encoding the endonuclease is operably linked. In some embodiments, delivering the engineered nucleic acid editing system to said target nucleic acid locus comprises delivering a capped mRNA containing the open reading frame encoding the endonuclease. In some embodiments, delivering the engineered nucleic acid editing system to the target nucleic acid locus comprises delivering a translated polypeptide. In some embodiments, delivering the engineered nucleic acid editing system to the target nucleic acid locus comprises delivering a deoxyribonucleic acid (DNA) encoding the engineered guide ribonucleic acid structure operably linked to a ribonucleic acid (RNA) pol III promoter.

In some aspects, the present disclosure provides an engineered nucleic acid editing polypeptide, comprising: an endonuclease comprising a RuvC domain and an HNH domain, wherein the endonuclease is derived from an uncultivated microorganism, wherein the endonuclease is a class 2, type II Cas endonuclease, and wherein the RuvC domain lacks nuclease activity; and a base editor coupled to the endonuclease. In some embodiments, the endonuclease comprises a sequence with at least 95% sequence identity to any one of SEQ ID NOs: 21-23.

In some aspects, the present disclosure provides an engineered nucleic acid editing polypeptide, comprising: an endonuclease having at least 95% sequence identity to any one of SEQ ID NOs: 21-23, wherein the endonuclease comprises a RuvC domain lacking nuclease activity; and a base editor coupled to the endonuclease.

In some aspects, the present disclosure provides an engineered nucleic acid editing polypeptide, comprising: an endonuclease configured to bind to a protospacer adjacent motif (PAM) sequence comprising SEQ ID NOs: 145-147, wherein the endonuclease is a class 2, type II Cas endonuclease, and wherein the endonuclease comprises a RuvC domain lacks nuclease activity; and a base editor coupled to the endonuclease.

In some embodiments, the endonuclease is derived from an uncultivated microorganism. In some embodiments, the endonuclease has less than 80% identity to a Cas9 endonuclease. In some embodiments, the endonuclease further comprises an HNH domain. In some embodiments, the tracr ribonucleic acid sequence comprises a sequence with at least 80% sequence identity to about 60 to 90 consecutive nucleotides selected from any one of SEQ ID NOs: 27-29. In some embodiments, the base editor comprises a sequence with at least 70%, 80%, 90% or 95% identity to any one of SEQ ID NOs: 1-7. In some embodiments, the base editor is an adenine deaminase. In some embodiments, the adenosine deaminase comprises a sequence with at least 95% identity to SEQ ID NO: 8. In some embodiments, the base editor is a cytosine deaminase. In some embodiments, the cytosine deaminase comprises a sequence with at least 95% identity to SEQ ID NO: 9. In some embodiments, the adenosine cytosine deaminase comprises a sequence with at least 95% identity to any one of SEQ ID NOs: 10-17.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 depicts typical organizations of CRISPR/Cas loci of different classes and types.

FIG. 2 shows the structure of a base editor plasmid containing a T7 promoter driving expression of the systems described herein.

FIG. 3 shows plasmid maps for systems described herein. MGA contains TadA*(from ABE8.17m)-SV40 NLS and MGC contains APOBEC1 (from BE3)-uracil glycosylase inhibitor-SV40 NLS.

FIG. 4 shows predicted catalytic residues on RuvCI domains from endonucleases described herein which are mutated to disrupt nuclease activity for nickase generation.

FIG. 5 depicts how single guide RNA expression cassettes are cloned into systems described herein. A fragment may comprise a T7 plus spacer and the other fragment comprises spacer plus single guide scaffold sequence plus bidirectional terminator may be assembled into expression plasmids, resulting functional constructs that may express sgRNAs and base editors simultaneously.

FIGS. 6A and 6B show sgRNA designs for lacZ targeting in E. coli. The spacer length used for the systems described herein was 22 nucleotides. For selected systems described herein, three sgRNAs targeting lacZ in E. coli were designed to determine editing windows.

FIG. 7 shows results of a selected mutated effectors examined for nickase activity. Purified nickases supplemented with their cognate sgRNAs were used to nick a 600 bp fluorophore labeled (6-FAM) double-stranded DNA fragment. Reacted DNA products were resolved by 10% TBE-Urea denaturing gel and nickase activity was determined by whether it shows bands of 600 and 200 bases. By contrast, wild type effectors showed bands of 400 and 200 bases.

FIGS. 8A and 8B show Sanger sequencing results demonstrating base edits by systems described herein.

FIG. 9 shows how the systems described herein expand base-editing capabilities with the endonucleases and base editors described herein.

### BRIEF DESCRIPTION OF THE SEQUENCE LISTING

The Sequence Listing filed herewith provides exemplary polynucleotide and polypeptide sequences for use in methods, compositions and systems according to the disclosure. Below are exemplary descriptions of sequences therein.

SEQ ID NOs: 1-3 show the full-length peptide sequences of MG66 deaminases suitable for the engineered nucleic acid editing systems described herein.

SEQ ID NOs: 4-7 show the full-length peptide sequences of MG67 deaminases suitable for the engineered nucleic acid editing systems described herein.

SEQ ID NOs: 8-17 show the sequences of reference deaminases.

SEQ ID NO: 18 shows the sequence of a reference uracil DNA glycosylase inhibitor.

SEQ ID NO: 19 shows the sequence of an adenine base editor.

SEQ ID NO: 20 shows the sequence of a cytosine base editor.

SEQ ID NOs: 21-23 show the full-length peptide sequences of MG nickases suitable for the engineered nucleic acid editing systems described herein.

SEQ ID NOs: 24-26 shows the protospacer and PAM used in in vitro nickase assays described herein.

SEQ ID NOs: 27-29 show the peptide sequences of single guide RNA used in in vitro nickase assays described herein.

SEQ ID NOs: 30-53 show the sequences of spacers when targeting E. coli lacZ.

SEQ ID NOs: 54-61 show the sequences of primers when conducting site directed mutagenesis.

SEQ ID NOs: 62-63 show the sequences of primers for lacZ sequencing.

SEQ ID NOs: 64-139 show the sequences of primers used during amplification.

SEQ ID NOs: 140-142 show the sequences of primers for lacZ sequencing.

SEQ ID NOs: 143-144 show the sequences of primers used during amplification.

SEQ ID NOs: 145-147 show protospacer adjacent motifs suitable for the engineered nucleic acid editing systems described herein.

SEQ ID NOs: 148-163 show nuclear localization sequences (NLS's) suitable for the engineered nucleic acid editing systems described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, techniques of immunology, biochemistry, chemistry, molecular biology, microbiology, cell biology, genomics and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which is entirely incorporated by reference herein).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

As used herein, a “cell” generally refers to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide,” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [αS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Labeling may also be carried out with quantum dots. Detectable labels may include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

The terms “transfection” or “transfected” generally refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88.

The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

As used herein, the “non-native” can generally refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that may also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The term “promoter”, as used herein, generally refers to the regulatory DNA region which controls transcription or expression of a gene and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic necessary elements to promote transcriptional expression of an operably linked polynucleotide. Eukaryotic basal promoters typically, though not necessarily, contain a TATA-box and/or a CAAT box.

The term “expression”, as used herein, generally refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refer to juxtaposition of genetic elements, e.g., a promoter, an enhancer, a polyadenylation sequence, etc., wherein the elements are in a relationship permitting them to operate in the expected manner. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein, “an expression cassette” and “a nucleic acid cassette” are used interchangeably generally to refer to a combination of nucleic acid sequences or elements that are expressed together or are operably linked for expression. In some cases, an expression cassette refers to the combination of regulatory elements and a gene or genes to which they are operably linked for expression.

A “functional fragment” of a DNA or protein sequence generally refers to a fragment that retains a biological activity (either functional or structural) that is substantially similar to a biological activity of the full-length DNA or protein sequence. A biological activity of a DNA sequence may be its ability to influence expression in a manner known to be attributed to the full-length sequence.

As used herein, an “engineered” object generally indicates that the object has been modified by human intervention. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

As used herein, “synthetic” and “artificial” are used interchangeably to refer to a protein or a domain thereof that has low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains.

The term “tracrRNA” or “tracr sequence”, as used herein, can generally refer to a nucleic acid with at least about 5%, 10%, 20%, 30%, 40%, 50%, 60%, 70%, 80%, 90%, 95%, or 100% sequence identity and/or sequence similarity to a wild type exemplary tracrRNA sequence (e.g., a tracrRNA from S. pyogenes S. aureus, etc.). tracrRNA can refer to a nucleic acid with at most about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes S. aureus, etc.). tracrRNA may refer to a modified form of a tracrRNA that can comprise a nucleotide change such as a deletion, insertion, or substitution, variant, mutation, or chimera. A tracrRNA may refer to a nucleic acid that can be at least about 60% identical to a wild type exemplary tracrRNA (e.g., a tracrRNA from S. pyogenes S. aureus, etc.) sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA (e.g., a tracrRNA from S. pyogenes S. aureus, etc.) sequence over a stretch of at least 6 contiguous nucleotides. Type II tracrRNA sequences can be predicted on a genome sequence by identifying regions with complementarity to part of the repeat sequence in an adjacent CRISPR array.

As used herein, a “guide nucleic acid” can generally refer to a nucleic acid that may hybridize to another nucleic acid. A guide nucleic acid may be RNA. A guide nucleic acid may be DNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence.” A nucleic acid-targeting segment may comprise a sub-segment that may be referred to as a “protein binding segment” or “protein binding sequence” or “Cas protein binding segment”.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at blast.ncbi.nlm.nih.gov); CLUSTALW with parameters of; the Smith-Waterman homology search algorithm with parameters of a match of 2, a mismatch of −1, and a gap of −1; MUSCLE with default parameters; MAFFT with parameters retree of 2 and maxiterations of 1000; Novafold with default parameters; HMMER hmmalign with default parameters.

As used herein, the term “RuvC_III domain” generally refers to a third discontinuous segment of a RuvC endonuclease domain (the RuvC nuclease domain being comprised of three discontiguous segments, RuvC_I, RuvC_II, and RuvC_III). A RuvC domain or segments thereof can generally be identified by alignment to known domain sequences, structural alignment to proteins with annotated domains, or by comparison to Hidden Markov Models (HMMs) built based on known domain sequences (e.g., Pfam HMM PF18541 for RuvC_III).

As used herein, the term “HNH domain” generally refers to an endonuclease domain having characteristic histidine and asparagine residues. An HNH domain can generally be identified by alignment to known domain sequences, structural alignment to proteins with annotated domains, or by comparison to Hidden Markov Models (HMMs) built based on known domain sequences (e.g., Pfam HMM PF01844 for domain HNH).

As used herein, the term “base editor” generally refers to an enzyme that catalyzes the conversion of one target base or base pair into another (e.g. A:T to G:C, C:G to T:A) without requiring the creation and repair of a double-strand break. In some embodiments, the base editor is a deaminase.

As used herein, the term “deaminase” generally refers to a protein or enzyme that catalyzes a deamination reaction. In some embodiments, the deaminase is an adenosine deaminase, which catalyzes the hydrolytic deamination of adenine or adenosine (e.g., an engineered adenosine deaminase that deaminates adenosine in DNA). In some embodiments, the deaminase or deaminase domain is a cytidine (or cytosine) deaminase, catalyzing the hydrolytic deamination of cytidine (or cytosine) or deoxycytidine to uridine (or uracil) or deoxyuridine, respectively. In some embodiments, the deaminase or deaminase domain is a cytidine (or cytosine) deaminase domain, catalyzing the hydrolytic deamination of cytosine (or cytosine) to uracil (or uridine). In some embodiments, the deaminase or deaminase domain is a naturally-occurring deaminase from an organism, such as a human, chimpanzee, gorilla, monkey, cow, dog, rat, mouse, or bacterium (e.g. E. coli). In some embodiments, the deaminase or deaminase domain is a variant of a naturally-occurring deaminase from an organism that does not occur in nature.

The term “optimally aligned” in the context of two or more nucleic acids or polypeptide sequences, generally refers to two (e.g., in a pairwise alignment) or more (e.g., in a multiple sequence alignment) sequences that have been aligned to maximal correspondence of amino acids residues or nucleotides, for example, as determined by the alignment producing a highest or “optimized” percent identity score.

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues) without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of the endonuclease protein sequences described herein. In some embodiments, such conservatively substituted variants are functional variants. Such functional variants can encompass sequences with substitutions such that the activity of one or more critical active site residues or guide RNA binding residues of the endonuclease are not disrupted.

Also included in the current disclosure are variants of any of the enzymes described herein with substitution of one or more catalytic residues to decrease or eliminate activity of the enzyme (e.g. decreased-activity variants). In some embodiments, a decreased activity variant as a protein described herein comprises a disrupting substitution of at least one, at least two, or all three catalytic residues. In some embodiments, any of the endonucleases described herein can comprise a nickase mutation. In some embodiments, any of the endonucleases described herein can comprise a RuvC domain lacking nuclease activity. In some embodiments, any of the endonucleases described herein can be configured to cleave one strand of a double-stranded target deoxyribonucleic acid. In some embodiments, any of the endonucleases described herein can comprise can be configured to lack endonuclease activity.

Conservative substitution tables providing functionally similar amino acids are available from a variety of references (see, for e.g., Creighton, Proteins: Structures and Molecular Properties (W H Freeman & Co.; 2nd edition (December 1993)). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

Overview

The discovery of new Cas enzymes with unique functionality and structure may offer the potential to further disrupt deoxyribonucleic acid (DNA) editing technologies, improving speed, specificity, functionality, and ease of use. Relative to the predicted prevalence of Clustered Regularly Interspaced Short Palindromic Repeats (CRISPR) systems in microbes and the sheer diversity of microbial species, comparatively few functionally characterized CRISPR/Cas enzymes exist in the literature. This is partly because a huge number of microbial species may not be readily cultivated in laboratory conditions. Metagenomic sequencing from natural environmental niches that represent large numbers of microbial species may offer the potential to drastically increase the number of new CRISPR/Cas systems known and speed the discovery of new oligonucleotide editing functionalities. A recent example of the fruitfulness of such an approach is demonstrated by the 2016 discovery of CasX/CasY CRISPR systems from metagenomic analysis of natural microbial communities.

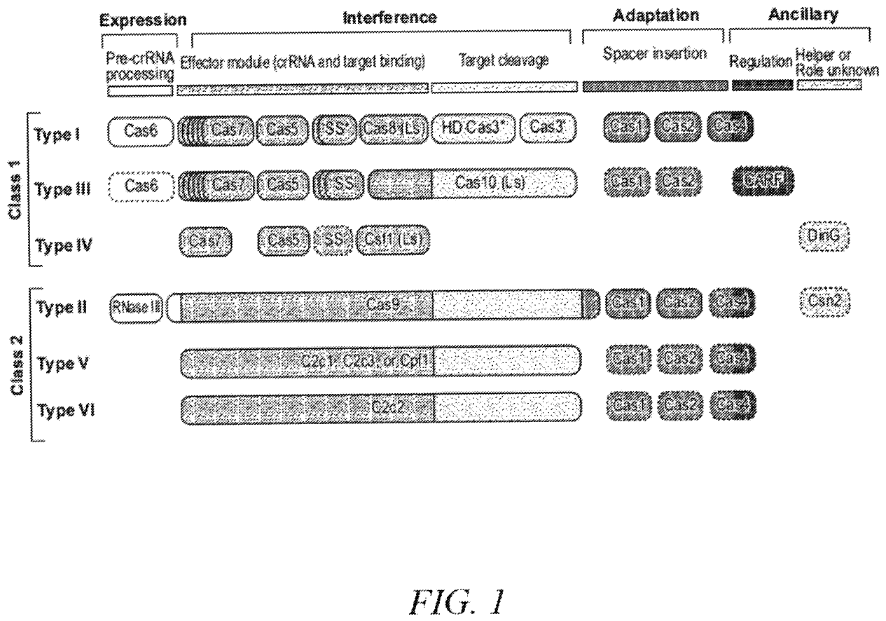

CRISPR/Cas systems are RNA-directed nuclease complexes that have been described to function as an adaptive immune system in microbes. In their natural context, CRISPR/Cas systems occur in CRISPR (clustered regularly interspaced short palindromic repeats) operons or loci, which generally comprise two parts: (i) an array of short repetitive sequences (30-40 bp) separated by equally short spacer sequences, which encode the RNA-based targeting element; and (ii) ORFs encoding the Cas encoding the nuclease polypeptide directed by the RNA-based targeting element alongside accessory proteins/enzymes. Efficient nuclease targeting of a particular target nucleic acid sequence generally requires both (i) complementary hybridization between the first 6-8 nucleic acids of the target (the target seed) and the crRNA guide; and (ii) the presence of a protospacer-adjacent motif (PAM) sequence within a defined vicinity of the target seed (the PAM usually being a sequence not commonly represented within the host genome). Depending on the exact function and organization of the system, CRISPR-Cas systems are commonly organized into 2 classes, 5 types and 16 subtypes based on shared functional characteristics and evolutionary similarity (see FIG. 1).

Class I CRISPR-Cas systems have large, multisubunit effector complexes, and comprise Types I, III, and IV.

Type I CRISPR-Cas systems are considered of moderate complexity in terms of components. In Type I CRISPR-Cas systems, the array of RNA-targeting elements is transcribed as a long precursor crRNA (pre-crRNA) that is processed at repeat elements to liberate short, mature crRNAs that direct the nuclease complex to nucleic acid targets when they are followed by a suitable short consensus sequence called a protospacer-adjacent motif (PAM). This processing occurs via an endoribonuclease subunit (Cas6) of a large endonuclease complex called Cascade, which also comprises a nuclease (Cas3) protein component of the crRNA-directed nuclease complex. Cas I nucleases function primarily as DNA nucleases.

Type III CRISPR systems may be characterized by the presence of a central nuclease, known as Cas10, alongside a repeat-associated mysterious protein (RAMP) that comprises Csm or Cmr protein subunits. Like in Type I systems, the mature crRNA is processed from a pre-crRNA using a Cas6-like enzyme. Unlike type I and II systems, type III systems appear to target and cleave DNA-RNA duplexes (such as DNA strands being used as templates for an RNA polymerase).

Type IV CRISPR-Cas systems possess an effector complex that consists of a highly reduced large subunit nuclease (csf1), two genes for RAMP proteins of the Cas5 (csf3) and Cas7 (csf2) groups, and, in some cases, a gene for a predicted small subunit; such systems are commonly found on endogenous plasmids.

Class II CRISPR-Cas systems generally have single-polypeptide multidomain nuclease effectors, and comprise Types II, V and VI.

Type II CRISPR-Cas systems are considered the simplest in terms of components. In Type II CRISPR-Cas systems, the processing of the CRISPR array into mature crRNAs does not require the presence of a special endonuclease subunit, but rather a small trans-encoded crRNA (tracrRNA) with a region complementary to the array repeat sequence; the tracrRNA interacts with both its corresponding effector nuclease (e.g. Cas9) and the repeat sequence to form a precursor dsRNA structure, which is cleaved by endogenous RNAse III to generate a mature effector enzyme loaded with both tracrRNA and crRNA. Cas II nucleases are known as DNA nucleases. Type 2 effectors generally exhibit a structure consisting of a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand.

Type V CRISPR-Cas systems are characterized by a nuclease effector (e.g. Cas12) structure similar to that of Type II effectors, comprising a RuvC-like domain. Similar to Type II, most (but not all) Type V CRISPR systems use a tracrRNA to process pre-crRNAs into mature crRNAs; however, unlike Type II systems which requires RNAse III to cleave the pre-crRNA into multiple crRNAs, type V systems are capable of using the effector nuclease itself to cleave pre-crRNAs. Like Type-II CRISPR-Cas systems, Type V CRISPR-Cas systems are again known as DNA nucleases. Unlike Type II CRISPR-Cas systems, some Type V enzymes (e.g., Cas12a) appear to have a robust single-stranded nonspecific deoxyribonuclease activity that is activated by the first crRNA directed cleavage of a double-stranded target sequence.

Type VI CRIPSR-Cas systems have RNA-guided RNA endonucleases. Instead of RuvC-like domains, the single polypeptide effector of Type VI systems (e.g. Cas13) comprises two HEPN ribonuclease domains. Differing from both Type II and V systems, Type VI systems also appear to not need a tracrRNA for processing of pre-crRNA into crRNA. Similar to type V systems, however, some Type VI systems (e.g., C2C2) appear to possess robust single-stranded nonspecific nuclease (ribonuclease) activity activated by the first crRNA directed cleavage of a target RNA.

Because of their simpler architecture, Class II CRISPR-Cas have been most widely adopted for engineering and development as designer nuclease/genome editing applications.

One of the early adaptations of such a system for in vitro use can be found in Jinek et al. (Science. 2012 Aug. 17; 337(6096):816-21, which is entirely incorporated herein by reference). The Jinek study first described a system that involved (i) recombinantly-expressed, purified full-length Cas9 (e.g., a Class II, Type II Cas enzyme) isolated from S. pyogenes SF370, (ii) purified mature ˜42 nt crRNA bearing a ˜20 nt 5′ sequence complementary to the target DNA sequence desired to be cleaved followed by a 3′ tracr-binding sequence (the whole crRNA being in vitro transcribed from a synthetic DNA template carrying a T7 promoter sequence); (iii) purified tracrRNA in vitro transcribed from a synthetic DNA template carrying a T7 promoter sequence, and (iv) Mg²⁺. Jinek later described an improved, engineered system wherein the crRNA of (ii) is joined to the 5′ end of (iii) by a linker (e.g., GAAA) to form a single fused synthetic guide RNA (sgRNA) capable of directing Cas9 to a target by itself.

Mali et al. (Science. 2013 Feb. 15; 339(6121): 823-826), which is entirely incorporated herein by reference, later adapted this system for use in mammalian cells by providing DNA vectors encoding (i) an ORF encoding codon-optimized Cas9 (e.g., a Class II, Type II Cas enzyme) under a suitable mammalian promoter with a C-terminal nuclear localization sequence (e.g., SV40 NLS) and a suitable polyadenylation signal (e.g., TK pA signal); and (ii) an ORF encoding an sgRNA (having a 5′ sequence beginning with G followed by 20 nt of a complementary targeting nucleic acid sequence joined to a 3′ tracr-binding sequence, a linker, and the tracrRNA sequence) under a suitable Polymerase III promoter (e.g., the U6 promoter).

Base Editing

Base editing is the conversion of one target base or base pair into another (e.g. A:T to G:C, C:G to T:A) without requiring the creation and repair of a double-strand break. The base editing may be achieved with the help of DNA and RNA base editors that allow the introduction of point mutations at specific sites, in either DNA or RNA. Generally, DNA base editors may comprise a fusion of a catalytically inactive nuclease and a catalytically active base-modification enzyme that acts only on single-stranded DNAs (ssDNAs). RNA base editors may comprise of similar, RNA-specific enzymes. Base editing may increase the efficiency of gene modification, while reducing the off-target and random mutations in the DNA.

DNA base editors are engineered ribonucleoprotein complexes that act as tools for single base substitution in cells and organism. They may be created by fusing an engineered base-modification enzyme and a catalytically deficient Cas variant that cannot cut dsDNA, but it is able to unfold the dsDNA in a protospacer adjacent motif (PAM) sequence-dependent manner, such that a guide RNA can find its complementary target to indicate a ssDNA scission site. The guide RNA anneals to the complementary DNA, displacing a fragment of ssDNA and directing the Cas ‘scissors’ to the base modification site. The cellular repair machinery will repair the nicked non-edited strand using information from the complementary edited template.

So far, two types of DNA editors, cytosine base (CBEs) and adenine base editors (ABEs) have been developed. They were shown to efficiently and precisely edit point mutations in DNA with minimal off-target DNA editing (see Nat Biotechnol. 2017; 35:435-437, Nat Biotechnol. 2017; 35:438-440 and Nat Biotechnol. 2017; 35:475-480, each of which is entirely incorporated herein by reference). However, recent findings indicate that off-target modifications are present in DNA, and that many off-target modifications are also introduced into RNA by DNA base editors.

MG Base Editors

In some aspects, the present disclosure provides for an engineered nucleic acid editing system, comprising: (a) an endonuclease comprising a RuvC domain and an HNH domain, wherein the endonuclease is derived from an uncultivated microorganism, wherein the endonuclease is a class 2, type II Cas endonuclease, and wherein the RuvC domain lacks nuclease activity; (b) a base editor coupled to the endonuclease; and (c) an engineered guide ribonucleic acid structure configured to form a complex with the endonuclease comprising: (i) a guide ribonucleic acid sequence configured to hybridize to a target deoxyribonucleic acid sequence; and (ii) a tracr ribonucleic acid sequence configured to bind to the endonuclease. In some embodiments, the endonuclease comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of SEQ ID NOs: 21-23 or a variant thereof.

In some aspects, the present disclosure provides for an engineered nucleic acid editing system comprising: (a) an endonuclease having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of SEQ ID NOs: 21-23 or a variant thereof, wherein the endonuclease comprises a RuvC domain lacking nuclease activity; a base editor coupled to the endonuclease; and an engineered guide ribonucleic acid structure configured to form a complex with the endonuclease comprising: (i) a guide ribonucleic acid sequence configured to hybridize to a target deoxyribonucleic acid sequence; and (ii) a tracr ribonucleic acid sequence configured to bind to the endonuclease.

In some aspects, the present disclosure provides for an engineered nucleic acid editing system comprising: (a) an endonuclease configured to bind to a protospacer adjacent motif (PAM) sequence comprising SEQ ID NOs: 145-147, wherein the endonuclease is a class 2, type II Cas endonuclease, and wherein the endonuclease comprises a RuvC domain lacks nuclease activity; and (b) a base editor coupled to the endonuclease; and (c) an engineered guide ribonucleic acid structure configured to form a complex with the endonuclease comprising: (i) a guide ribonucleic acid sequence configured to hybridize to a target deoxyribonucleic acid sequence; and (ii) a tracr ribonucleic acid sequence configured to bind to the endonuclease.

In some embodiments, the endonuclease is derived from an uncultivated microorganism. In some embodiments, the endonuclease has less than 80% identity to a Cas9 endonuclease. In some embodiments, the endonuclease further comprises an HNH domain. In some embodiments, the tracr ribonucleic acid sequence comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to about 60 to 90 consecutive non-degenerate nucleotides from any one of SEQ ID NOs: 27-29, or a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to non-degenerate nucleotides of any one of SEQ ID NOs: 27-29.

In some aspects, the present disclosure provides an engineered nucleic acid editing system comprising, (a) an engineered guide ribonucleic acid structure comprising: (i) a guide ribonucleic acid sequence configured to hybridize to a target deoxyribonucleic acid sequence; and (ii) a tracr ribonucleic acid sequence configured to bind to an endonuclease, wherein the tracr ribonucleic acid sequence comprises a sequence with at least 80% sequence identity to about 60 to 90 consecutive nucleotides selected from any one of SEQ ID NOs: 27-29; and a class 2, type II Cas endonuclease configured to bind to the engineered guide ribonucleic acid.

In some embodiments, the endonuclease is configured to bind to a protospacer adjacent motif (PAM) sequence selected from the group consisting of SEQ ID NOs: 145-147. In some embodiments, the base editor comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any one of SEQ ID NOs: 1-17. In some embodiments, the base editor is an adenine deaminase. In some embodiments, the adenosine deaminase comprises a sequence with at least 95% identity to SEQ ID NO: 8. In some embodiments, the base editor is a cytosine deaminase. In some embodiments, the cytosine deaminase comprises a sequence with at least 95% identity to SEQ ID NO: 9. In some embodiments, the cytosine deaminase comprises a sequence with at least 95% identity to any one of SEQ ID NOs: 10-17.

In some embodiments, the engineered nucleic acid editing system further comprises a uracil DNA glycosylase inhibitor. In some embodiments, the uracil DNA glycosylase inhibitor comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 18 or a variant thereof.

In some embodiments, the engineered guide ribonucleic acid structure comprises at least two ribonucleic acid polynucleotides. In some embodiments, the engineered guide ribonucleic acid structure comprises one ribonucleic acid polynucleotide comprising the guide ribonucleic acid sequence and the tracr ribonucleic acid sequence. In some embodiments, the guide ribonucleic acid sequence is complementary to a prokaryotic, bacterial, archaeal, eukaryotic, fungal, plant, mammalian, or human genomic sequence. In some embodiments, the guide ribonucleic acid sequence is 15-24 nucleotides in length. In some embodiments, the endonuclease comprises one or more nuclear localization sequences (NLSs) proximal to an N- or C-terminus of said endonuclease.

The NLS can comprise any of the sequences in Table 1 below, or a combination thereof:

TABLE 1
Example NLS Sequences that can be used with Cas Effectors According to the
Disclosure

| Source | NLS amino acid sequence | SEQ ID NO: |
|---|---|---|
| SV40 | PKKKRKV | 148 |
| nucleoplasmin bipartite NLS | KRPAATKKAGQAKKKK | 149 |
| c-myc NLS | PAAKRVKLD | 150 |
| c-myc NLS | RQRRNELKRSP | 151 |
| hRNPA1 M9 NLS | NQSSNFGPMKGGNFGGRSSGPYGGGGQYFAK | 152 |
| | PRNQGGY | |
| Importin-alpha IBB domain | RMRIZFKNKGKDTAELRRRRVEVSVELRKAK | 153 |
| | KDEQILKRRNV | |
| Myoma T protein | VSRKRPRP | 154 |
| Myoma T protein | PPKKARED | 155 |
| p53 | PQPKKKPL | 156 |
| mouse c-abl IV | SALIKKKKKMAP | 157 |
| influenza virus NS1 | DRLRR | 158 |
| influenza virus NS1 | PKQKKRK | 159 |
| Hepatitis virus delta antigen | RKLKKKIKKL | 160 |
| mouse Mx1 protein | REKKKFLKRR | 161 |
| human poly (ADP-ribose) | KRKGDEVDGVDEVAKKKSKK | 162 |
| polymerase | | |
| steroid hormone receptor (human) | RKCLQAGMNLEARKTKK | 163 |
| glucocorticoid | | |

In some embodiments, the endonuclease is covalently coupled directly to the base editor or covalently coupled to the base editor through a linker. In some embodiments, a polypeptide comprises the endonuclease and the base editor. In some embodiments, the endonuclease is configured to cleave one strand of a double-stranded target deoxyribonucleic acid. In some embodiments, the system further comprises a source of Mg²⁺.

In some embodiments, the endonuclease comprises a sequence at least 70%, at least 80%, or at least 90% identical to SEQ ID NO: 21; the guide RNA structure comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 27; and the endonuclease is configured to bind to a PAM comprising SEQ ID NO: 145.

In some embodiments, the endonuclease comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 22; the guide RNA structure comprises a sequence at least 70%, at least 80%, or at least 90% identical to SEQ ID NO: 28; and the endonuclease is configured to bind to a PAM comprising SEQ ID NO: 146.

In some embodiments, the endonuclease comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 23; the guide RNA structure comprises a sequence at least 70%, at least 80%, or at least 90% identical to SEQ ID NO: 29; and the endonuclease is configured to bind to a PAM comprising SEQ ID NO: 147.

In some embodiments, the base editor comprises an adenine deaminase. In some embodiments, the adenine deaminase comprises SEQ ID NO: 8. In some embodiments, the base editor comprises a cytosine deaminase. In some embodiments, the cytosine deaminase comprises SEQ ID NO: 9. In some embodiments, the engineered nucleic acid editing system described herein further comprises a uracil DNA glycosylation inhibitor. In some embodiments, the uracil DNA glycosylation inhibitor comprises SEQ ID NO: 18.

In some embodiments, the sequence identity is determined by a BLASTP, CLUSTALW, MUSCLE, MAFFT, or Smith-Waterman homology search algorithm. In some embodiments, the sequence identity is determined by said BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some aspects, the present disclosure provides a nucleic acid comprising an engineered nucleic acid sequence optimized for expression in an organism, wherein the nucleic acid encodes a class 2, type II Cas endonuclease coupled to a base editor, and wherein the endonuclease is derived from an uncultivated microorganism.

In some aspects, the present disclosure provides a nucleic acid comprising an engineered nucleic acid sequence optimized for expression in an organism, wherein the nucleic acid encodes an endonuclease having at least 70% sequence identity to any one of SEQ ID NOs: 21-23 coupled to a base editor. In some embodiments, the endonuclease comprises a sequence encoding one or more nuclear localization sequences (NLSs) proximal to an N- or C-terminus of said endonuclease. In some embodiments, the NLS comprises a sequence with sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of SEQ ID NOs: 148-163. In some embodiments, the organism is prokaryotic, bacterial, eukaryotic, fungal, plant, mammalian, rodent, or human.

In some aspects, the present disclosure provides a vector comprising a nucleic acid sequence encoding a class 2, type II Cas endonuclease coupled to a base editor, wherein said endonuclease is derived from an uncultivated microorganism. In some embodiments, the vector comprises the nucleic acid described herein. In some embodiments, the vector further comprises a nucleic acid encoding an engineered guide ribonucleic acid structure configured to form a complex with the endonuclease comprising: a guide ribonucleic acid sequence configured to hybridize to a target deoxyribonucleic acid sequence; and a tracr ribonucleic acid sequence configured to binding to the endonuclease. In some embodiments, the vector is a plasmid, a minicircle, a CELiD, an adeno-associated virus (AAV) derived virion, or a lentivirus. In some aspects, the present disclosure provides a cell comprising the vector described herein. In some aspects, the present disclosure provides a method of manufacturing an endonuclease, comprising cultivating the cell described herein.

In some aspects, the present disclosure provides a method for modifying a double-stranded deoxyribonucleic acid polynucleotide comprising contacting the double-stranded deoxyribonucleic acid polynucleotide with a complex comprising: an endonuclease comprising a RuvC domain and an HNH domain, wherein the endonuclease is derived from an uncultivated microorganism, wherein the endonuclease is a class 2, type II Cas endonuclease, and wherein the RuvC domain lacks nuclease activity; a base editor coupled to the endonuclease; and an engineered guide ribonucleic acid structure configured to bind to the endonuclease and the double-stranded deoxyribonucleic acid polynucleotide; wherein the double-stranded deoxyribonucleic acid polynucleotide comprises a protospacer adjacent motif (PAM).

In some embodiments, the endonuclease comprising a RuvC domain and an HNH domain is covalently coupled directly to the base editor or covalently coupled to the base editor through a linker. In some embodiments, the endonuclease comprising a RuvC domain and an HNH domain comprises a sequence with sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of SEQ ID NOs: 21-23 or a variant thereof.

In some aspects, the present disclosure provides a method for modifying a double-stranded deoxyribonucleic acid polynucleotide, comprising contacting the double-stranded deoxyribonucleic acid polynucleotide with a complex comprising: a class 2, type II Cas endonuclease, a base editor coupled to the endonuclease, and an engineered guide ribonucleic acid structure configured to bind to the endonuclease and the double-stranded deoxyribonucleic acid polynucleotide; wherein the double-stranded deoxyribonucleic acid polynucleotide comprises a protospacer adjacent motif (PAM); and wherein the PAM comprises a sequence selected from the group consisting of SEQ ID NOs: 145-147.

In some embodiments, the class 2, type II Cas endonuclease is covalently coupled to the base editor or coupled to the base editor through a linker. In some embodiments, the base editor comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NOs: 1-17. In some embodiments, the base editor comprises an adenine deaminase; the double-stranded deoxyribonucleic acid polynucleotide comprises an adenine; and modifying the double-stranded deoxyribonucleic acid polypeptide comprises converting the adenine to guanine. In some embodiments, the adenine deaminase comprises a sequence with at least 95% identity to SEQ ID NO: 8.

In some embodiments, the base editor comprises a cytosine deaminase; the double-stranded deoxyribonucleic acid polynucleotide comprises a cytosine; and modifying the double-stranded deoxyribonucleic acid polypeptide comprises converting the cytosine to uracil. In some embodiments, the cytosine deaminase comprises a sequence with at least 95% identity to SEQ ID NO: 9. In some embodiments, the cytosine deaminase comprises a sequence with at least 95% identity to any one of SEQ ID NOs: 10-17.

In some embodiments, the complex further comprises a uracil DNA glycosylase inhibitor. In some embodiments, the uracil DNA glycosylase inhibitor comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 18. In some embodiments, the double-stranded deoxyribonucleic acid polynucleotide comprises a first strand comprising a sequence complementary to a sequence of the engineered guide ribonucleic acid structure and a second strand comprising said PAM. In some embodiments, the PAM is directly adjacent to the 3′ end of the sequence complementary to the sequence of the engineered guide ribonucleic acid structure.

In some embodiments, the class 2, type II Cas endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas 13d endonuclease. In some embodiments, the class 2, type II Cas endonuclease is derived from an uncultivated microorganism. In some embodiments, the double-stranded deoxyribonucleic acid polynucleotide is a eukaryotic, plant, fungal, mammalian, rodent, or human double-stranded deoxyribonucleic acid polynucleotide.

In some aspects, the present disclosure provides a method of modifying a target nucleic acid locus, said method comprising delivering to said target nucleic acid locus the engineered nucleic acid editing system described herein, wherein the endonuclease is configured to form a complex with the engineered guide ribonucleic acid structure, and wherein the complex is configured such that upon binding of the complex to the target nucleic acid locus, the complex modifies a nucleotide of the target nucleic locus.

In some embodiments, the engineered nucleic acid editing system comprises an adenine deaminase, the nucleotide is an adenine, and modifying the target nucleic acid locus comprises converting the adenine to a guanine. In some embodiments, the engineered nucleic acid editing system comprises a cytidine deaminase and a uracil DNA glycosylase inhibitor, the nucleotide is a cytosine and modifying the target nucleic acid locus comprises converting the adenine to a uracil. In some embodiments, the target nucleic acid locus comprises genomic DNA, viral DNA, or bacterial DNA. In some embodiments, the target nucleic acid locus is in vitro. In some embodiments, the target nucleic acid locus is within a cell. In some embodiments, the cell is a prokaryotic cell, a bacterial cell, a eukaryotic cell, a fungal cell, a plant cell, an animal cell, a mammalian cell, a rodent cell, a primate cell, or a human cell. In some embodiments, the cell is within an animal.

In some embodiments, the cell is within a cochlea. In some embodiments, the cell is within an embryo. In some embodiments, the embryo is a two-cell embryo. In some embodiments, the embryo is a mouse embryo. In some embodiments, delivering the engineered nucleic acid editing system to the target nucleic acid locus comprises delivering the nucleic acid described herein or the vector described herein. In some embodiments, delivering the engineered nucleic acid editing system to the target nucleic acid locus comprises delivering a nucleic acid comprising an open reading frame encoding the endonuclease.

In some embodiments, the nucleic acid comprises a promoter to which the open reading frame encoding the endonuclease is operably linked. In some embodiments, delivering the engineered nucleic acid editing system to said target nucleic acid locus comprises delivering a capped mRNA containing the open reading frame encoding the endonuclease. In some embodiments, delivering the engineered nucleic acid editing system to the target nucleic acid locus comprises delivering a translated polypeptide. In some embodiments, delivering the engineered nucleic acid editing system to the target nucleic acid locus comprises delivering a deoxyribonucleic acid (DNA) encoding the engineered guide ribonucleic acid structure operably linked to a ribonucleic acid (RNA) pol III promoter.

In some aspects, the present disclosure provides an engineered nucleic acid editing polypeptide, comprising: an endonuclease comprising a RuvC domain and an HNH domain, wherein the endonuclease is derived from an uncultivated microorganism, wherein the endonuclease is a class 2, type II Cas endonuclease, and wherein the RuvC domain lacks nuclease activity; and a base editor coupled to the endonuclease. In some embodiments, the endonuclease comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of SEQ ID NOs: 21-23 or a variant thereof.

In some aspects, the present disclosure provides an engineered nucleic acid editing polypeptide, comprising: an endonuclease having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of SEQ ID NOs: 21-23 or a variant thereof, wherein the endonuclease comprises a RuvC domain lacking nuclease activity; and a base editor coupled to the endonuclease.

In some aspects, the present disclosure provides an engineered nucleic acid editing polypeptide, comprising: an endonuclease configured to bind to a protospacer adjacent motif (PAM) sequence comprising SEQ ID NOs: 145-147, wherein the endonuclease is a class 2, type II Cas endonuclease, and wherein the endonuclease comprises a RuvC domain lacks nuclease activity; and a base editor coupled to the endonuclease.

In some embodiments, the endonuclease is derived from an uncultivated microorganism. In some embodiments, the endonuclease has less than 80% identity to a Cas9 endonuclease. In some embodiments, the endonuclease further comprises an HNH domain. In some embodiments, the tracr ribonucleic acid sequence comprises a sequence with at least 80% sequence identity to about 60 to 90 consecutive nucleotides selected from any one of SEQ ID NOs: 27-29. In some embodiments, the base editor comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of SEQ ID NOs: 1-17 or a variant thereof. In some embodiments, the base editor is an adenine deaminase. In some embodiments, the adenosine deaminase comprises a sequence with at least 95% identity to SEQ ID NO: 8. In some embodiments, the base editor is a cytosine deaminase. In some embodiments, the cytosine deaminase comprises a sequence with at least 95% identity to SEQ ID NO: 9. In some embodiments, the adenosine cytosine deaminase comprises a sequence with at least 95% identity to any one of SEQ ID NOs: 10-17.

Systems of the present disclosure may be used for various applications, such as, for example, nucleic acid editing (e.g., gene editing), binding to a nucleic acid molecule (e.g., sequence-specific binding). Such systems may be used, for example, for addressing (e.g., removing or replacing) a genetically inherited mutation that may cause a disease in a subject, inactivating a gene in order to ascertain its function in a cell, as a diagnostic tool to detect disease-causing genetic elements (e.g. via cleavage of reverse-transcribed viral RNA or an amplified DNA sequence encoding a disease-causing mutation), as deactivated enzymes in combination with a probe to target and detect a specific nucleotide sequence (e.g. sequence encoding antibiotic resistance int bacteria), to render viruses inactive or incapable of infecting host cells by targeting viral genomes, to add genes or amend metabolic pathways to engineer organisms to produce valuable small molecules, macromolecules, or secondary metabolites, to establish a gene drive element for evolutionary selection, to detect cell perturbations by foreign small molecules and nucleotides as a biosensor.

TABLE 2
Sequence Listing of Protein and Nucleic Acid Sequences Referred to Herein

| | SEQ | | | | | |
|---|---|---|---|---|---|---|
| | ID | | | | Other | |
| Category | NO: | Description | Type | Organism | Information | Sequence |
| MG66 | 1 | MG66-1 | protein | unknown | uncultivated | MKRIVTGTNDSCPLTGALEYAKVDKY |
| putative | | deaminase | | | organism | TRQTLASMGITRMEDFKAFEEHQFLS |
| cytidine | | | | | | QPGIGPKRFSQVTALMEAFNLRWKKP |
| deaminase | | | | | | DPMNRPGWDEYFTRIASVVATRSHDE |
| | | | | | | QTQVGCVVVDRDRRIRATGYNGFPPG |
| | | | | | | FPDDALPKTRPEKYPFMVHAELNAIA |
| | | | | | | SSNQSLKRCTLYCTHSPCGNCVKAII |
| | | | | | | TTGIERVVFRTAYTDWEESRKLLDLG |
| | | | | | | RLEYYMISD |
| MG66 | 2 | MG66-16 | protein | unknown | uncultivated | MEMTKEYTYSSGYNVIDNEIDFKCKF |
| putative | | deaminase | | | organism | NEEWVSYFMNIARVVATKSKDPNTKV |
| cytidine | | | | | | GCVVVDTKTKRIMATGYNGFPPGVNE |
| deaminase | | | | | | DKSRWERPTKYDFVTHAEANCIAAAA |
| | | | | | | RFGIGLAGATMFVTLHPCVDCAKLIA |
| | | | | | | SAGISNIAFIESELERKQDRDWITHL |
| | | | | | | ENAKAIFRESRINLIAIREVKPIADV |
| | | | | | | LEPQPLSFKGSVATVDDLNTIDPIIG |
| | | | | | | DVFYVNSENTEYVYWLGKWGLLHETF |
| | | | | | | KDPNRFPYKQ |
| MG66 | 3 | MG66-17 | protein | unknown | uncultivated | MKNSESHPIQSKLERPGWDRYFMDIA |
| putative | | deaminase | | | organism | KAVSARSIDPSVHVGAVIVNSGRRIL |
| cytidine | | | | | | STGYNGFPPGFPDDELPLTRPEKYPY |
| deaminase | | | | | | TVHAEVNAIASSQSDLRGGTLYCTLS |
| | | | | | | PCVECTKVIITSGIQCVVFEEKYATS |
| | | | | | | E |
| MG67 | 4 | MG67-1 | protein | unknown | uncultivated | MKVQVVNEIGATEIQIKSYLTLCKNA |
| putative | | deaminase | | | organism | QKAMQNAYSEYSGFKVGAALLLKNGE |
| cytidine | | | | | | VVQSCNVENASYGLTMCAERAAIAKA |
| deaminase | | | | | | VSEGFKPGDLDAIAISSNGKDFSPCG |
| | | | | | | ACRQVIHEFGVDVVVIFEFEGNVVIT |
| | | | | | | TASKMLPYGYSEEKQS |
| MG67 | 5 | MG67-3 | protein | unknown | uncultivated | MKEAYNGLLQHAHRAKENAHAPYSGF |
| putative | | deaminase | | | organism | KVGAAILAKDGRIIEGCNVENVAYGS |
| cytidine | | | | | | TMCAERVAIYKAVSMGYKPGDFDAIA |
| deaminase | | | | | | IASTGKNFSPCGACRQVINEFGDDIN |
| | | | | | | IIFEWEEKIVTESAKSMLPYNFESQT |
| | | | | | | ASPHHKS |
| MG67 | 6 | MG67-5 | protein | unknown | uncultivated | MKEAYKELLQHAHRAKENAHAPYSGF |
| putative | | deaminase | | | organism | KVGAAILAKDGQIIEGCNVENVAYGS |
| cytidine | | | | | | TMCAERVAIYKAVSMGYKPGDFDAIA |
| deaminase | | | | | | IASTGKNFSPCGSCRQVINEFGDDID |
| | | | | | | IIFEWEEKIVTESAKSMLPYNFESQT |
| | | | | | | ASPHHKS |
| MG67 | 7 | MG67-6 | protein | unknown | uncultivated | MKEEYKALLQHAHQAKENAHAPYSGF |
| putative | | deaminase | | | organism | KVGAAILAKDGRIIEGCNVENVAYGS |
| cytidine | | | | | | TMCAERVAIYKAVSMGYKPGDFDAIA |
| deaminase | | | | | | IASTGKNFSPCGACRQVINEFGDDIN |
| | | | | | | IIFEWEEKIVTESAKSMLPYNFESQT |
| | | | | | | ASPHHKS |
| reference | 8 | P68398 | protein | Escherichia | | MSEVEFSHEYWMRHALTLAKRAWDER |
| deaminase | | TADA | | coli | | EVPVGAVLVHNNRVIGEGWNRPIGRH |
| | | tRNA | | strain K12 | | DPTAHAEIMALRQGGLVMQNYRLIDA |
| | | specific | | OX | | TLYVTLEPCVMCAGAMIHSRIGRVVF |
| | | adenosine | | | | GARDAKTGAAGSLMDVLHHPGMNHRV |
| | | deaminase | | | | EITEGILADECAALLSDFFRMRRQEI |
| | | | | | | KAQKKAQSSTD |
| reference | 9 | P38483 | protein | Rattus | | MSSETGPVAVDPTLRRRIEPHEFEVF |
| deaminase | | APOBEC 1 | | norvegicus | | FDPRELRKETCLLYEINWGGRHSIWR |
| | | C U editing | | | | HTSQNTNKHVEVNFIEKFTTERYFCP |
| | | deaminase | | | | NTRCSITWFLSWSPCGECSRAITEFL |
| | | | | | | SRYPHVTLFIYIARLYHHADPRNRQG |
| | | | | | | LRDLISSGVTIQIMTEQESGYCWRNF |
| | | | | | | VNYSPSNEAHWPRYPHLWVRLYVLEL |
| | | | | | | YCIILGLPPCLNILRRKQPQLTFFTI |
| | | | | | | ALQSCHYQRLPPHILWATGLK |
| reference | 10 | Aicda XM | protein | Hetero- | | MAVGSKPKAALVGPHWERERIWCFLC |
| deaminase | | 004869540 | | cephalus | | STGLGTQQTGQTSRWLRPAATQDPVS |
| | | cytidine | | glaber | | PPRSLLMKQRKFLYHFKNVRWAKGRH |
| | | deaminase | | | | ETYLCYVVKRRDSATSFSLDFGYLRN |
| | | | | | | KSGCHVELLFLRYISDWDLDPGRCYR |
| | | | | | | VTWFTSWSPCYDCARHVADFLRGNPN |
| | | | | | | LSLRIFTARLYFCEDRKAEPEGLRRL |
| | | | | | | HRAGVQIAIMTFKDYFYCWNTFVENH |
| | | | | | | ERTFKAWEGLHENSVRLSRQLRRILL |
| | | | | | | PLYEVDDLRDAFRTLGL |
| reference | 11 | PmCDA1 | protein | Petromyzon | | MAGYECVRVSEKLDFDTFEFQFENLH |
| deaminase | | L1 | | marinus | | YATERHRTYVIFDVKPQSAGGRSRRL |
| | | AVN88313.1 | | | | WGYIINNPNVCHAELILMSMIDRHLE |
| | | cytidine | | | | SNPGVYAMTWYMSWSPCANCSSKLNP |
| | | deaminase | | | | WLKNLLEEQGHTLTMHFSRIYDRDRE |
| | | | | | | GDHRGLRGLKHVSNSFRMGVVGRAEV |
| | | | | | | KECLAEYVEASRRTLTWLDTTESMAA |
| | | | | | | KMRRKLFCILVRCAGMRESGIPLHLF |
| | | | | | | TLQTPLLSGRVVWWRV |
| reference | 12 | PmCDA1 | protein | Petromyzon | | MTDAEYVRIHEKLDIYTFKKQFFNNK |
| deaminase | | ABO15149.1 | | marinus | | KSVSHRCYVLFELKRRGERRACFWGY |
| | | cytosine | | | | AVNKPQSGTERGIHAEIFSIRKVEEY |
| | | deaminase | | | | LRDNPGQFTINWYSSWSPCADCAEKI |
| | | | | | | LEWYNQELRGNGHTLKIWACKLYYEK |
| | | | | | | NARNQIGLWNLRDNGVGLNVMVSEHY |
| | | | | | | QCCRKIFIQSSHNQLNENRWLEKTLK |
| | | | | | | RAEKRRSELSIMIQVKILHTTKSPAV |
| reference | 3 | NP | protein | Homo | | MEASPASGPRHLMDPHIFTSNFNNGI |
| deaminase | | 663745.1 | | sapiens | | GRHKTYLCYEVERLDNGTSVKMDQHR |
| | | DNA dC- | | | | GFLHNQAKNLLCGFYGRHAELRFLDL |
| | | dU-editing | | | | VPSLQLDPAQIYRVTWFISWSPCFSW |
| | | deaminase | | | | GCAGEVRAFLQENTHVRLRIFAARIY |
| | | APOBEC- | | | | DYDPLYKEALQMLRDAGAQVSIMTYD |
| | | 3A isoform | | | | EFKHCWDTFVDHQGCPFQPWDGLDEH |
| | | a | | | | SQALSGRLRAILQNQGN |
| reference | 14 | Q9GZX7.1 | protein | Homo | | MDSLLMNRRKFLYQFKNVRWAKGRRE |
| deaminase | | AICDA | | sapiens | | TYLCYVVKRRDSATSFSLDFGYLRNK |
| | | Single- | | | | NGCHVELLFLRYISDWDLDPGRCYRV |
| | | stranded | | | | TWFTSWSPCYDCARHVADFLRGNPNL |
| | | DNA | | | | SLRIFTARLYFCEDRKAEPEGLRRLH |
| | | cytosine | | | | RAGVQIAIMTFKDYFYCWNTEVENHE |
| | | deaminase | | | | RTFKAWEGLHENSVRLSRQLRRILLP |
| | | (Activation- | | | | LYEVDDLRDAFRTLGL |
| | | induced | | | | |
| | | cytidine | | | | |
| | | deaminase, | | | | |
| | | Cytidine | | | | |
| | | aminohydro- | | | | |
| | | lase) | | | | |
| reference | 15 | LpCDA1L13 | protein | Lampetra | | MAGDENVRVSEKLHFNTFEFEFENLH |
| deaminase | | AVN88320.1 | | planeri | | YAEGRGQTYVIFDVKPQSEGGRGERL |
| | | cytidine | | | | WGYVRNKPLGDHAEVILMSKINDHLE |
| | | deaminase | | | | THQDNYTMTWYMSWSPCGKCSSELVP |
| | | | | | | WLQNLLKKQHKLTMHFSRIYDKDRAV |
| | | | | | | DHRGLCDLQHVVSNGFQMGVMGQTEV |
| | | | | | | DTCLAEYVEASGCPPLKWLHMTDSNA |
| | | | | | | TQMQDKLSSILMNRF |
| reference | 16 | LpCDA1L11 | protein | Lampetra | | MAGDENVRVSKKLDFNTFEFEFENLH |
| deaminase | | AVN88319.1 | | planeri | | YAEGRGRTYVIFDVKPQSEGGRGERL |
| | | cytidine | | | | WGYVRNNPLDDHAEVILMSKINDHLE |
| | | deaminase | | | | THQGNYTMTWYMSWSPCGNCSSELVP |
| | | | | | | WLQNLLEEQQHTLTMYFSRIYDKDRA |
| | | | | | | VDHRGLCDLQHVVSNGFQMGVMGQTE |
| | | | | | | VDTCLAEYVEASGCPPLKWLHMTDSN |
| | | | | | | ATQTQDKLSSILMNRF |
| reference | 17 | ljCDA1 | nucleo- | Lampetra | | GCCAACGCCGAGTACGTGCGCGTGGG |
| deaminase | | cytidine | tide | planeri | | CGAGAAGCTGGACAGCTGCACCTTCC |
| | | deaminase | | | | GCACCCAGTTCCTGAACTACCGCCGC |
| | | | | | | AGCCGCAGCCGCCGCTGCTGCGTGAT |
| | | | | | | CTTCGAGCTGAAGCGCCAGAACAGCC |
| | | | | | | GCGTGCGCTTCTGGGGCTACGCCATG |
| | | | | | | AACAAGCCCTGGAGCAACGCCGACGT |
| | | | | | | GGGCATCCACGCCGAGTTCTTCTGCA |
| | | | | | | TCAAGAAGGTGAAGAAGTACCTGCGC |
| | | | | | | AAGAACCCCGGCATCTACACCATCAA |
| | | | | | | CTGGTACAGCAGCTGGAGCCCCTGCG |
| | | | | | | CCAACTGCGCCGAGAAGATCCTGAAC |
| | | | | | | TGGTACAACAAGAAGCTGATGGGCAA |
| | | | | | | GGGCCACACCCTGAAGATtTGGGCCT |
| | | | | | | GCAAGCTGTACTTCGAGAACATCAAG |
| | | | | | | CGCAACCAGATCGGCCTGTGGAACCT |
| | | | | | | GCGCAACAACGGCGTGGGCCTGGCCA |
| | | | | | | TCATGCTGGGCGAGCACTACCAGTGG |
| | | | | | | TGCTGGAACAACTACATCCAGACCCT |
| | | | | | | GGGCCGCAACCTGAACGAGAACAAGT |
| | | | | | | GGCTGAAaAAGACCAGCAACCGCGCC |
| | | | | | | CGCACCCGCCGCAGCGAGCTGAGCAT |
| | | | | | | CATGATCCACGTGAAGCGCCTGCACA |
| | | | | | | CCGCCCGCCTGCTGCTGTTCAAGCGC |
| | | | | | | CTGTGCGGCTGGTTCAGC |
| reference | 18 | P14739 | protein | Bacillus | | MTNLSDIIEKETGKQLVIQESILMLP |
| UGI | | UNGI | | phage | | EEVEEVIGNKPESDILVHTAYDESTD |
| | | BPPB2 | | PBS2 | | ENVMLLTSDAPEYKPWALVIQDSNGE |
| | | (UGI) | | | | NKIKML |
| adenine | 19 | linker-His | protein | artificial | | MGSSHHHHHHSEVEFSHEYWMRHALT |
| base | | tag-adenine | | sequence | | LAKRARDEREVPVGAVLVLNNRVIGE |
| editor | | deaminse- | | | | GWNRAIGLHDPTAHAEIMALRQGGLV |
| | | linker- | | | | MQNYRLIDATLYSTFEPCVMCAGAMI |
| | | nickase- | | | | HSRIGRVVFGVRNAKTGAAGSLMDVL |
| | | linker-SV40 | | | | HYPGMNHRVEITEGILADECAALLCY |
| | | NLS | | | | FFRMPRRVFNAQKKAQSSTDSGGSSG |
| | | | | | | GSSGSETPGTSESATPESSGGSSGGS |
| | | | | | | MKKKVFAFALGKASIGYCVRDGFNIN |
| | | | | | | EANSIIIDKDHAETMSLRDRRRIKKT |
| | | | | | | LDAHHAREEYFNKVWSGVGLNILDKN |
| | | | | | | NNNFKKEFPSKNEDVIYTSCLLRIAL |
| | | | | | | LQNQKLEEWQIYKALYNAFQRRGYDV |
| | | | | | | NIAWKSVQTDDDKENIERMKKYTCEN |
| | | | | | | DIELIISDEYKYPCYYDALRLGLWNE |
| | | | | | | DEPTKLNRSIPQHNSNKVRTTEYVAP |
| | | | | | | RELVEKELKQLWLNAQLQLPQLNSIS |
| | | | | | | AEEFLYGEYKIPYGSCVQKEFKQYRG |
| | | | | | | TKKDWQGVLCQKIPRFDNRIIAKCKL |
| | | | | | | LPKRNVCKANTIENVSFVLLMKLKNL |
| | | | | | | RFTVISGEKLRLSPVQIKQIYENWLE |
| | | | | | | KVEDTYNKDLEKAEKLGKENPEKRLN |
| | | | | | | ITITRQEIEKIIGEKIIDKIEPMKAD |
| | | | | | | ISGRSSFCRRACQIMNKIILDGELYP |
| | | | | | | QNVDISEFVDRDNAKNGITEEEIQSM |
| | | | | | | LSKIGDWNNLYIPDNREENAQNASNS |
| | | | | | | RIKTDIMLGNITNPIVRNRLQIFRDL |
| | | | | | | LLNLAEKYGTPDEVIFEFVRDGADNS |
| | | | | | | LYGSKKAQDTERFMKQQEKENEQIIN |
| | | | | | | ELKDAGLYVNGQNHANATYFLMHKLL |
| | | | | | | KSQGGKCIYSGQNISPSNYDECEIDH |
| | | | | | | TYPRTLGGNDALYNKVVCYRRENQDK |
| | | | | | | KGRTPYEWLSPDSDKWANYVNRLNKI |
| | | | | | | KKQLGKKKFELLTSKPEDCEKLIESY |
| | | | | | | NGLAETSHIARVAQQMTAFIFGWGLQ |
| | | | | | | IQGESRRIFVNNGSSTCAIRKRYGLN |
| | | | | | | KLLGDNLKKNRSNDKHHALDAICISF |
| | | | | | | SRDFKYDKEIKKDIIKGFNPEIVKNA |
| | | | | | | IDKIMPYPYANDKPFKGNTKPLETIY |
| | | | | | | GLRTYGDKSYITQRVELNSIDKKATK |
| | | | | | | IKSIIDETIKNDLLNKLKENPTEQEW |
| | | | | | | KLMLQNYIHPKKQTKVKKVMISVSEG |
| | | | | | | EITKDSNNRERMGEFVDFGTKGTQHQ |
| | | | | | | FKHSKRHKGQILYFNEKGVVEVMPVY |
| | | | | | | SNIKTTDVKDKLQNMGCKLYNKGQMF |
| | | | | | | YSGCLVDIPKPFKAGSKEYPAGRYQI |
| | | | | | | KTIRSDKVAELEDACGNKISTNVKYL |
| | | | | | | VPAEFKKVESKGSGGSPKKKRKV |
| cytosine | 20 | linker-His | protein | artificial | | MGSSHHHHHHMSSETGPVAVDPTLRR |
| base | | tag-cytidine | | sequence | | RIEPHEFEVFFDPRELRKETCLLYEI |
| editor | | deaminase- | | | | NWGGRHSIWRHTSQNTNKHVEVNFIE |
| | | linker- | | | | KFTTERYFCPNTRCSITWFLSWSPCG |
| | | nickase- | | | | ECSRAITEFLSRYPHVTLFIYIARLY |
| | | linker- | | | | HHADPRNRQGLRDLISSGVTIQIMTE |
| | | uracil | | | | QESGYCWRNFVNYSPSNEAHWPRYPH |
| | | glycosylase | | | | LWVRLYVLELYCIILGLPPCLNILRR |
| | | inhibitor- | | | | KQPQLTFFTIALQSCHYQRLPPHILW |
| | | linker-SV40 | | | | ATGLKSGSETPGTSESATPESAMKKK |
| | | NLS | | | | VFAFALGKASIGYCVRDGFNINEANS |
| | | | | | | IIIDKDHAETMSLRDRRRIKKTLDAH |
| | | | | | | HAREEYFNKVWSGVGLNILDKNNNNF |
| | | | | | | KKEFPSKNEDVIYTSCLLRIALLQNQ |
| | | | | | | KLEEWQIYKALYNAFQRRGYDVNIAW |
| | | | | | | KSVQTDDDKENIERMKKYTCENDIEL |
| | | | | | | IISDEYKYPCYYDALRLGLWNEDEPT |
| | | | | | | KLNRSIPQHNSNKVRTTEYVAPRELV |
| | | | | | | EKELKQLWLNAQLQLPQLNSISAEEF |
| | | | | | | LYGEYKIPYGSCVQKEFKQYRGTKKD |
| | | | | | | WQGVLCQKIPRFDNRIIAKCKLLPKR |
| | | | | | | NVCKANTIENVSFVLLMKLKNLRFTV |
| | | | | | | ISGEKLRLSPVQIKQIYENWLEKVED |
| | | | | | | TYNKDLEKAEKLGKENPEKRLNITIT |
| | | | | | | RQEIEKIIGEKIIDKIEPMKADISGR |
| | | | | | | SSFCRRACQIMNKIILDGELYPQNVD |
| | | | | | | ISEFVDRDNAKNGITEEEIQSMLSKI |
| | | | | | | GDWNNLYIPDNREENAQNASNSRIKT |
| | | | | | | DIMLGNITNPIVRNRLQIFRDLLLNL |
| | | | | | | AEKYGTPDEVIFEFVRDGADNSLYGS |
| | | | | | | KKAQDTERFMKQQEKENEQIINELKD |
| | | | | | | AGLYVNGQNHANATYFLMHKLLKSQG |
| | | | | | | GKCIYSGQNISPSNYDECEIDHIYPR |
| | | | | | | TLGGNDALYNKVVCYRRENQDKKGRT |
| | | | | | | PYEWLSPDSDKWANYVNRLNKIKKQL |
| | | | | | | GKKKFELLTSKPEDCEKLIESYNGLA |
| | | | | | | ETSHIARVAQQMTAFIFGWGLQIQGE |
| | | | | | | SRRIFVNNGSSTCAIRKRYGLNKLLG |
| | | | | | | DNLKKNRSNDKHHALDAICISFSRDF |
| | | | | | | KYDKEIKKDIIKGFNPEIVKNAIDKI |
| | | | | | | MPYPYANDKPFKGNTKPLETIYGLRT |
| | | | | | | YGDKSYITQRVELNSIDKKATKIKSI |
| | | | | | | IDETIKNDLLNKLKENPTEQEWKLML |
| | | | | | | QNYIHPKKQTKVKKVMISVSEGEITK |
| | | | | | | DSNNRERMGEFVDFGTKGTQHQFKHS |
| | | | | | | KRHKGQILYFNEKGVVEVMPVYSNIK |
| | | | | | | TTDVKDKLQNMGCKLYNKGQMFYSGC |
| | | | | | | LVDIPKPFKAGSKEYPAGRYQIKTIR |
| | | | | | | SDKVAELEDACGNKISTNVKYLVPAE |
| | | | | | | FKKVESKGSGGSTNLSDIIEKETGKQ |
| | | | | | | LVIQESILMLPEEVEEVIGNKPESDI |
| | | | | | | LVHTAYDESTDENVMLLTSDAPEYKP |
| | | | | | | WALVIQDSNGENKIKMLSGGSPKKKR |
| | | | | | | KV |
| nickase | 21 | nMG4-2 | protein | artificial | | MLREPGNSVKSKIMGQQAKRRSYVLG |
| | | (D28A) | | sequence | | LAIGTHSVGWALLKFRDGRPCGVERA |
| | | nickase | | | | GVRIFEPGVEEVAFERGRAEPPGQKR |
| | | | | | | RQARALRRQTERRARRKAKLLHILQR |
| | | | | | | AGLLPKGEADEILPALDRDILARHSA |
| | | | | | | AWPGARDALPYWLRSGALDHRLEPHE |
| | | | | | | FGRALYHLGQRRGFLSNRRAPMRKNE |
| | | | | | | EDGKVKAGISTLKEQMEKAGARTLGE |
| | | | | | | FFAGLDPHQERIRQRYTSREMYEQEF |
| | | | | | | EAVWSAQAAHHPAILTDDLKARVHHA |
| | | | | | | VFHQRPLHNQSYLAGSCTLEPDRKRT |
| | | | | | | PWACLIAQRFRMLQKLNDTRVLPASG |
| | | | | | | PERPLSDEERQTVLTELDRKKELKFD |
| | | | | | | RVRKLLGLSADSSFNWESGGEDRLVG |
| | | | | | | NTTNARLAKVFGKRWWSLSPDDRDQV |
| | | | | | | VEDVRSYEKAEALARRGREHWGLDEK |
| | | | | | | AAGELSKLSLEDGYCRLSRQAIERLL |
| | | | | | | PGMEKGTAYMELVRKLYPDRWAAGKP |
| | | | | | | VDLLPALAETDLDMRNPVVRRCLTEL |
| | | | | | | RKVVNAVVRHYGKPSAIRIELARDLR |
| | | | | | | KSAKQREQTWRRNRRNQQDREAAAEK |
| | | | | | | LLQEARIANPSRADVEKVLLAEECGW |
| | | | | | | HCPYTGHGFGMADLFGPHPHFDVEHI |
| | | | | | | VPFSRSLDNSFLNKTICEARENRDRK |
| | | | | | | RNHTPYEAYGADAERWDQIIARVQSF |
| | | | | | | RGTASREKLRRFQQHEVEDLDGVAQR |
| | | | | | | ELNDTRYASLLAVQYVGMLYGGAVDA |
| | | | | | | GHVRRVQAAKGGTTGYLRDMYGLGFV |
| | | | | | | LGEGRKERSDHRHHAVDAVAIALTDP |
| | | | | | | AALKSISQAASDERRGGRVSFGAVAL |
| | | | | | | PWVDFIGDVQAAIEAINVSHRPSRKV |
| | | | | | | NGALHEETFYGPRGMDGDGRPTGYVQ |
| | | | | | | RKPVERLSAKEIPNIPDPAVREAVQA |
| | | | | | | KLDEVGGTPAQAFKDPANHPVRKRGI |
| | | | | | | PVHKVRLRLNINPVQVGSGATERHVL |
| | | | | | | TGSNHHMEIIEVRDAKGGKKWTGRLV |
| | | | | | | HRLEAKRRALGRETIVDRAVQAGRQF |
| | | | | | | QFSLSPGDMIELTGEDGERKLHVVRS |
| | | | | | | ISEGRIEYVDARDARKKADIRASGDW |
| | | | | | | RKPAVGSLLRLHCRKVVVTPFGEIRY |
| | | | | | | AND |
| nickase | 22 | nMG7-1 | protein | artificial | | MSNKTILGLALGVSSIGWAIIERNDE |
| | | (D10A) | | sequence | | NGRIVKSGVRVIPSSKSELSVFKDFD |
| | | nickase | | | | KGKPASFSKERTEKRGIRRSYFRKKL |
| | | | | | | RRAKLIEHLKENNMFDPELLGPKYSI |
| | | | | | | DVWEWREKATKEKITLAQLGRVLLHI |
| | | | | | | NQKRGYKSNRKAIVDEESDSNWLNAI |
| | | | | | | NDNSKLLREKGITVGEYFYQEGKLHE |
| | | | | | | RKPKVKFALHFRMKVRIFNRKDYLDE |
| | | | | | | IEQIWKKQSEFYPELTDELKESIIDH |
| | | | | | | TIFYQRPLKSAKHLLSECRYEKMHKV |
| | | | | | | IARSNPLFQLFRVLEKVNNLRAEDAF |
| | | | | | | GNNREITDEEKLKIIEACTSAQSWKL |
| | | | | | | LDKKKNLSKSKIKSILGLGKDYEINL |
| | | | | | | DSIEGSKTLHSIWEVLMKSWGEAGDW |
| | | | | | | IDFDWSIQGNDFSKQKSYQLWHALYS |
| | | | | | | IDEPQYLRKKLCEGFGFDLDTARLLM |
| | | | | | | NIRLESDYGALSARAIKRIIPELLKF |
| | | | | | | PKDATKAIENAGYKFTDSETKEERES |
| | | | | | | RELKDRIEHLKKGALRNPVVEKVLNQ |
| | | | | | | LVTLVNAIYAHPELPNPDEIRVELAR |
| | | | | | | ELKSGAKERRRAELGMARAAKDNDRI |
| | | | | | | RELLQTEFGIPYPGRRDILRYRFWEE |
| | | | | | | QDMRCVYSGDVIPRNKLFVGEEYELD |
| | | | | | | HIIPRARLFNDSNSNLVLVKSSENKD |
| | | | | | | KSDMTAADYMKSKGEKAFEEYLVRVK |
| | | | | | | NLYDKGAKKKAGERGSGINKGKRNFL |
| | | | | | | IMKKEEIPQDFIERQLRESQYIVKEA |
| | | | | | | VKLLKEVCRDVTTTTGKITDLLKHQW |
| | | | | | | GANDVFRNIQVPKYRKWGMTETIVDR |
| | | | | | | KTGEVIERIIDWSKRKDHRHHALDAI |
| | | | | | | IVACTRQSYIQQLNRLNVLYENDYES |
| | | | | | | LKSYRKFELPWPSFHNDLISSLESLL |
| | | | | | | VSFRNKRRVATMNKNRIKVGGKKKYI |
| | | | | | | VQKTLTPRDAFHLETTYGRRLVNNYK |
| | | | | | | LVKLNKKFSMELAELVIDPDLKEKIL |
| | | | | | | NRLMEFGNDPQKAFANLKKNPFKWKN |
| | | | | | | ENLEEVLIYDEVFTTRKKLDEKFNNP |
| | | | | | | SEIIDPEVREIVTQRLKEFDNNPKKA |
| | | | | | | FADIENKPVWYNKDKQIRIKTVNTRA |
| | | | | | | KASDLYPVRTKENGNPKDFVFTRNNH |
| | | | | | | HITVYQKEDGKYYDKVTSFWEAFELK |
| | | | | | | KAKMPIYKENDDAAKAVLHLKINDMV |
| | | | | | | LVDLNPEDLDQNDPEFFNTLSEHLYR |
| | | | | | | VQKLASGDVTFRHHLETELSNKNTEV |
| | | | | | | RVTNAESLYNRVVMYPLDVLGLPK |
| nickase | 23 | nMG16-1 | protein | artificial | | MIKNILGLALGVGSIGWALIQTEDDQ |
| | | (D9A) | | sequence | | PKQIIGMGSRIVPLTKDDSDQFTKGQ |
| | | nickase | | | | AISKNAERTARRTTRKGYDRYQLRRA |
| | | | | | | LLTQVLRQNGMLPECMDENMIDLWKL |
| | | | | | | RSDAATEGKQLTLQQIGRVLYHINQK |
| | | | | | | RGYKHAKSDDNGDSKQTKYVEAVNLR |
| | | | | | | YKEIQEKNVTVGQHFYAELLNSKVES |
| | | | | | | GNGPYYTFRIKDKVFPRAAYIAEFDQ |
| | | | | | | IMGVQKEYYPNVLTDELIETLRNRII |
| | | | | | | FYQRPLKSCKHLVGLCEFEMRPYKKD |
| | | | | | | GKIVYGGPKCAPRTSPLAQLCAMWET |
| | | | | | | VNNITLTNRNNERLEISNEQRRQLVQ |
| | | | | | | FLCTHETLKLTDLYKILGITKKDGWY |
| | | | | | | GGKAIGKGIKGNVTLNQLRKALDGKY |
| | | | | | | SQWLEMPIERIDVVDRNTAEAFWAVS |
| | | | | | | PKVEETPLFQLWHAVYSLQNVEELTK |
| | | | | | | TLQNRFSITDPQVIDALCKIDFVKPG |
| | | | | | | YANKSHKFIRRLLPYLMEGMMYSEAC |
| | | | | | | ACIQINHSNSMTKAEREARPLAERIE |
| | | | | | | LLQKNALRQPVIEKILNQMINLVNRL |
| | | | | | | QQEYGPIDEARVELARELKQSREERK |
| | | | | | | DAFDRNNKNEKRNKEISALISEQGIR |
| | | | | | | PSRSRIQKYKMWEESEHRCMYCGKVV |
| | | | | | | NLSEFLNGADVEIEHIIPRSILFDDS |
| | | | | | | FSNKVCACRDCNREKDNMTAMDYMAS |
| | | | | | | KPEGEFEAYKQRVDEAFNAHRISKTK |
| | | | | | | RDHLLWRRADIPQDFIDRQLRLSQYI |
| | | | | | | ATKAVEILQQGIRQVWTSGGGVTDFL |
| | | | | | | RHQWGYDEILHTLNLPRYRQVEDLTE |
| | | | | | | MVHYEHAGQEHDEERIKNWSKRIDHR |
| | | | | | | HHAIDALTVALTRQSYIQRLNTLEAS |
| | | | | | | HEHMEKLVKEANTPYKEKKSLLEKWV |
| | | | | | | ALQPHFSVEEVTTQVDGILVSFRAGK |
| | | | | | | RVTTPARRAVYHGGKRTIVQRGIQVP |
| | | | | | | RGALTEDTIYGKLGDKFVVKYALDHP |
| | | | | | | SMKPENIVDPTIRLLVENRITALGKK |
| | | | | | | DAFKTPLYSAEGMEIKSVRCYTSLSE |
| | | | | | | KGVVPIKYNEKGNAIGFAKKGNNHHV |
| | | | | | | AIYKDQSGQYQEMVVSFWDAVERKLY |
| | | | | | | GVPTVITNPKTVWDELLEKELPQDFL |
| | | | | | | EKLPKDNWQYVLSMQENEMFVLGMEE |
| | | | | | | DEFNDAIDTQDYNTLNKHLYRVQKLS |
| | | | | | | HADYTFRFHTETKVDDKYDGVENGRN |
| | | | | | | TSMSLKALVRIRSFNGLFTQFPHKVK |
| | | | | | | IDIMGRITKA |
| target | 24 | nMG4-2 | nucleo- | artificial | | AGCCACCACGTCGCAAGCCTCGACTG |
| sequence | | (D28A) | tide | sequence | | TCCC |
| | | protospacer | | | | |
| | | and PAM | | | | |
| | | for in vitro | | | | |
| | | nickase | | | | |
| | | assay | | | | |
| target | 25 | nMG7-1 | nucleo- | artificial | | CCGTGAGCCACCACGTCGCAAGCCTC |
| sequence | | (D10A) | tide | sequence | | GACTAGTCGGG |
| | | protospacer | | | | |
| | | and PAM | | | | |
| | | for in vitro | | | | |
| | | nickase | | | | |
| | | assay | | | | |
| target | 26 | nMG16-1 | nucleo- | artificial | | CCGTGAGCCACCACGTCGCAAGCCTC |
| sequence | | (D9A) | tide | sequence | | GACTGGACCTG |
| | | protospacer | | | | |
| | | and PAM | | | | |
| | | for in vitro | | | | |
| | | nickase | | | | |
| | | assay | | | | |
| single | 27 | nMG4-2 | nucleo- | artificial | | CCACCACGTCGCAAGCCTCGACGCTG |
| guide | | (D28A) | tide | sequence | | TGGTTTGATGGGTTCGCTCCTCAAAT |
| RNA | | single guide | | | | CATAGTAAGGGGCAATTGCCTCGCGG |
| | | RNA for in | | | | GCTCTGCCCTACGGGGCACCCCTCGA |
| | | vitro nickase | | | | GCGCCTCCCAGTGGGGCGCTTTT |
| | | assay | | | | |
| single | 28 | nMG7-1 | nucleo- | artificial | | CCACCACGTCGCAAGCCTCGACGTTG |
| guide | | (D10A) | tide | sequence | | TGAATGGCTTTCAGAAATGAAGTTAT |
| RNA | | single guide | | | | TCACAATAAGGATTATTCCGTTGTGA |
| | | RNA for in | | | | AAACATTTAAAGCGGCCCTCGGGTCG |
| | | vitro nickase | | | | CTTTT |
| | | assay | | | | |
| single | 29 | nMG16-1 | nucleo- | artificial | | CCACCACGTCGCAAGCCTCGACGTTG |
| guide | | (D9A) | tide | sequence | | TGTATGGAAACATACACAATAAGGAT |
| RNA | | single guide | | | | TATTCCGTTGTGAAAACATTCAGGGT |
| | | RNA for in | | | | GGGACGCAAGTCTCGCCCTTTT |
| | | vitro nickase | | | | |
| | | assay | | | | |
| spacer | 30 | MGA4-2 | nucleo- | artificial | | TGATTAAATATGATGAAAACGG |
| | | sgRNA | tide | sequence | | |
| | | spacer 1 | | | | |
| | | (targeting E. | | | | |
| | | coli lacZ) | | | | |
| spacer | 31 | MGA4-2 | nucleo- | artificial | | AACGACATTGGCGTAAGTGAAG |
| | | sgRNA | tide | sequence | | |
| | | spacer 2 | | | | |
| | | (targeting E. | | | | |
| | | coli lacZ) | | | | |
| spacer | 32 | MGA4-2 | nucleo- | artificial | | ACGCGCGAATTGAATTATGGCC |
| | | sgRNA | tide | sequence | | |
| | | spacer 3 | | | | |
| | | (targeting E. | | | | |
| | | coli lacZ) | | | | |
| spacer | 33 | MGA7-1 | nucleo- | artificial | | GAAGATCAGGATATGTGGCGGA |
| | | sgRNA | tide | sequence | | |
| | | spacer 1 | | | | |
| | | (targeting E. | | | | |
| | | coli lacZ) | | | | |
| spacer | 34 | MGA7-1 | nucleo- | artificial | | AACGTCGAAAACCCGAAACTGT |
| | | sgRNA | tide | sequence | | |
| | | spacer 2 | | | | |
| | | (targeting E. | | | | |
| | | coli lacZ) | | | | |
| spacer | 35 | MGA7-1 | nucleo- | artificial | | AAACCCACGGCATGGTGCCAAT |
| | | sgRNA | tide | sequence | | |
| | | spacer 3 | | | | |
| | | (targeting E. | | | | |
| | | coli lacZ) | | | | |
| spacer | 36 | MGA16-1 | nucleo- | artificial | | GCCAGCTGGCGTAATAGCGAAG |
| | | sgRNA | tide | sequence | | |
| | | spacer 1 | | | | |
| | | (targeting E. | | | | |
| | | coli lacZ) | | | | |
| spacer | 37 | MGA16-1 | nucleo- | artificial | | AGGGTGAAACGCAGGTCGCCAG |
| | | sgRNA | tide | sequence | | |
| | | spacer 2 | | | | |
| | | (targeting E. | | | | |
| | | coli lacZ) | | | | |
| spacer | 38 | MGA16-1 | nucleo- | artificial | | CTACGTCTGAACGTCGAAAACC |
| | | sgRNA | tide | sequence | | |
| | | spacer 3 | | | | |
| | | (targeting E. | | | | |
| | | coli lacZ) | | | | |
| spacer | 39 | ABE8.17m | nucleo- | artificial | | TTTCTTTCACAGATGTGGAT |
| | | sgRNA | tide | sequence | | |
| | | spacer 1 | | | | |
| | | (targeting E. | | | | |
| | | coli lacZ) | | | | |
| spacer | 40 | ABE8.17m | nucleo- | artificial | | CAGGATATGTGGCGGATGAG |
| | | sgRNA | tide | sequence | | |
| | | spacer 2 | | | | |
| | | (targeting E. | | | | |
| | | coli lacZ) | | | | |
| spacer | 41 | ABE8.17m | nucleo- | artificial | | TGCGAATACGCCCACGCGAT |
| | | sgRNA | tide | sequence | | |
| | | spacer 3 | | | | |
| | | (targeting E. | | | | |
| | | coli lacZ) | | | | |
| spacer | 42 | MGC4-2 | nucleo- | artificial | | GCCGTGCGCTGTTCGCATTATC |
| | | sgRNA | tide | sequence | | |
| | | spacer 1 | | | | |
| | | (targeting E. | | | | |
| | | coli lacZ) | | | | |
| spacer | 43 | MGC4-2 | nucleo- | artificial | | ATACTGGCAGGCGTTTCGTCAG |
| | | sgRNA | tide | sequence | | |
| | | spacer 2 | | | | |
| | | (targeting E. | | | | |
| | | coli lacZ) | | | | |
| spacer | 44 | MGC4-2 | nucleo- | artificial | | CACTCCCCGCCGCGTCCCACGC |
| | | sgRNA | tide | sequence | | |
| | | spacer 3 | | | | |
| | | (targeting E. | | | | |
| | | coli lacZ) | | | | |
| spacer | 45 | MGC7-1 | nucleo- | artificial | | GCCGATCGCGTCACACTACGTC |
| | | sgRNA | tide | sequence | | |
| | | spacer 1 | | | | |
| | | (targeting E. | | | | |
| | | coli lacZ) | | | | |
| spacer | 46 | MGC7-1 | nucleo- | artificial | | GATCCGCGCTGGCTACCGGCGA |
| | | sgRNA | tide | sequence | | |
| | | spacer 2 | | | | |
| | | (targeting E. | | | | |
| | | coli lacZ) | | | | |
| spacer | 47 | MGC7-1 | nucleo- | artificial | | CCTTCCCGCCCGGTGCAGTATG |
| | | sgRNA | tide | sequence | | |
| | | spacer 3 | | | | |
| | | (targeting E. | | | | |
| | | coli lacZ) | | | | |
| spacer | 48 | MGC16-1 | nucleo- | artificial | | ACGTGACCTATCCCATTACGGT |
| | | sgRNA | tide | sequence | | |
| | | spacer 1 | | | | |
| | | (targeting E. | | | | |
| | | coli lacZ) | | | | |
| spacer | 49 | MGC16-1 | nucleo- | artificial | | ATCCGCCGTTTGTTCCCACGGA |
| | | sgRNA | tide | sequence | | |
| | | spacer 2 | | | | |
| | | (targeting E. | | | | |
| | | coli lacZ) | | | | |
| spacer | 50 | MGC16-1 | nucleo- | artificial | | CGACGGCACGCTGATTGAAGCA |
| | | sgRNA | tide | sequence | | |
| | | spacer 3 | | | | |
| | | (targeting E. | | | | |
| | | coli lacZ) | | | | |
| spacer | 51 | BE3 sgRNA | nucleo- | artificial | | CCTCTGGATGTCGCTCCACA |
| | | spacer 1 | tide | sequence | | |
| | | (targeting E. | | | | |
| | | coli lacZ) | | | | |
| spacer | 52 | BE3 sgRNA | nucleo- | artificial | | GGGACGCGCGAATTGAATTA |
| | | spacer 2 | tide | sequence | | |
| | | (targeting E. | | | | |
| | | coli lacZ) | | | | |
| spacer | 53 | BE3 sgRNA | nucleo- | artificial | | CGTTTTACAACGTCGTGACT |
| | | spacer 3 | tide | sequence | | |
| | | (targeting E. | | | | |
| | | coli lacZ) | | | | |
| primer | 54 | Site-directed | nucleo- | artificial | | CTCCTACGTTTTGGGCTTAGCGATCG |
| | | mutagenesis | tide | sequence | | GTACGCATA |
| | | of MG4-2 | | | | |
| | | (D28A) | | | | |
| primer | 55 | Site-directed | nucleo- | artificial | | CGGCGTTTGGCTTGTTGA |
| | | mutagenesis | tide | sequence | | |
| | | of MG4-2 | | | | |
| | | (D28A) | | | | |
| primer | 56 | Site-directed | nucleo- | artificial | | GTAATAAAACAATTTTAGGCCTGGCG |
| | | mutagenesis | tide | sequence | | TTAGGTGTTAGTTC |
| | | of MG7-1 | | | | |
| | | (D10A) | | | | |
| primer | 57 | Site-directed | nucleo- | artificial | | TCATGGTATATCTCCTTCTTAAAGTT |
| | | mutagenesis | tide | sequence | | |
| | | of MG7-1 | | | | |
| | | (D10A) | | | | |
| primer | 58 | Site-directed | nucleo- | artificial | | TGATTAAGAATATCTTGGGTCTGGCG |
| | | mutagenesis | tide | sequence | | CTGGGAGTCGGAAGC |
| | | of MG16-1 | | | | |
| | | (D9A) | | | | |
| primer | 59 | Site-directed | nucleo- | artificial | | TGGTATATCTCCTTCTTAAAGTTAAA |
| | | mutagenesis | tide | sequence | | C |
| | | of MG16-1 | | | | |
| | | (D9A) | | | | |
| primer | 60 | Site-directed | nucleo- | artificial | | GATAAGAAATACTCAATAGGACTGGC |
| | | mutagenesis | tide | sequence | | GATTGGCACAAATAGC |
| | | of SpCas9 | | | | |
| | | (D10A) | | | | |
| primer | 61 | Site-directed | nucleo- | artificial | | CATGGTATATCTCCTTCTTAAAGTT |
| | | mutagenesis | tide | sequence | | |
| | | of SpCas9 | | | | |
| | | (D10A) | | | | |
| primer | 62 | For lacZ | nucleo- | artificial | | CCAGGCTTTACACTTTATGCT |
| | | sequencing | tide | sequence | | |
| primer | 63 | For lacZ | nucleo- | artificial | | GTATGTGGTGGATGAAGCC |
| | | sequencing | tide | sequence | | |
| primer | 64 | Amplify the | nucleo- | artificial | | /56-FAM/AGCTTCCAGGGGGAAACG |
| | | fragment for | tide | sequence | | |
| | | nickase | | | | |
| | | assay | | | | |
| primer | 65 | Amplify the | nucleo- | artificial | | /56- |
| | | fragment for | tide | sequence | | FAM/ATTAACCTATAAAAATAGGCGT |
| | | nickase | | | | ATCA |
| | | assay | | | | |
| primer | 66 | Amplify T7 | nucleo- | artificial | | GATATACCATGGGCAGCAGTCATCAT |
| | | promoter- | tide | sequence | | CATCACCATCACTCCGAGGTTGAATT |
| | | His tag- | | | | TAGTCATG |
| | | adenine | | | | |
| | | deaminase | | | | |
| | | for MGA | | | | |
| | | entry | | | | |
| | | plasmid | | | | |
| primer | 67 | Amplify T7 | nucleo- | artificial | | AGACTTTCCTCTTCTTCTTGGGAGAC |
| | | promoter- | tide | sequence | | CCGCCAGAGCCTCGAGGACCCACCAG |
| | | His tag- | | | | AGCTGCCT |
| | | adenine | | | | |
| | | deaminase | | | | |
| | | for MGA | | | | |
| | | entry | | | | |
| | | plasmid | | | | |
| primer | 68 | Amplify | nucleo- | artificial | | TCTGGCGGGTCTCCCAAGAAGAAGAG |
| | | SV40 NLS- | tide | sequence | | GAAAGTCTAAGATCCGGCTGCTAACA |
| | | vector | | | | AAG |
| | | backbone | | | | |
| | | for MGA | | | | |
| | | entry | | | | |
| | | plasmid | | | | |
| primer | 69 | Amplify | nucleo- | artificial | | CTCCTTGCATGCTCTAGAATTAATTA |
| | | SV40 NLS- | tide | sequence | | AACCATTCCTTGCGGCGGC |
| | | vector | | | | |
| | | backbone | | | | |
| | | for MGA | | | | |
| | | entry | | | | |
| | | plasmid | | | | |
| primer | 70 | Amplify | nucleo- | artificial | | AGGAATGGTTTAATTAATTCTAGAGC |
| | | vector | tide | sequence | | ATGCAAGGAGATGGCGCCCAAC |
| | | backbone | | | | |
| | | for MGA | | | | |
| | | entry | | | | |
| | | plasmid | | | | |
| primer | 71 | Amplify | nucleo- | artificial | | GTGATGGTGATGATGATGACTGCTGC |
| | | vector | tide | sequence | | CCATGGTATATCTCCTTCTTAAAGTT |
| | | backbone | | | | AAAC |
| | | for MGA | | | | |
| | | entry | | | | |
| | | plasmid | | | | |
| primer | 72 | Amplify T7 | nucleo- | artificial | | AGGAGATATACCATGGGCAGCAGTCA |
| | | promoter- | tide | sequence | | TCATCATCACCATCACATGTCGTCTG |
| | | His-tag- | | | | AGACCGGG |
| | | cytosine | | | | |
| | | deaminase | | | | |
| | | for MGC | | | | |
| | | entry | | | | |
| | | plasmid | | | | |
| primer | 73 | Amplify T7 | nucleo- | artificial | | TAATATCTGAAAGATTGGTAGAACCA |
| | | promoter- | tide | sequence | | CCAGACCCGCGGCAGATTCAGGTGTC |
| | | His-tag- | | | | GCACTTTC |
| | | cytosine | | | | |
| | | deaminase | | | | |
| | | for MGC | | | | |
| | | entry | | | | |
| | | plasmid | | | | |
| primer | 74 | Amplify | nucleo- | artificial | | CGACACCTGAATCTGCCGCGGGTCTG |
| | | UGI-SV40 | tide | sequence | | GTGGTTCTACCAATCTTT |
| | | NLS for | | | | |
| | | MGC entry | | | | |
| | | plasmid | | | | |
| primer | 75 | Amplify | nucleo- | artificial | | TTAGACTTTCCTCTTCTTCTTGGGAG |
| | | UGI-SV40 | tide | sequence | | AACCACCAGAAAGCATTTTGATCTTG |
| | | NLS for | | | | TTTTCGCC |
| | | MGC entry | | | | |
| | | plasmid | | | | |
| primer | 76 | Amplify | nucleo- | artificial | | CTGGTGGTTCTCCCAAGAAGAAGAGG |
| | | SV40 NLS- | tide | sequence | | AAAGTCTAAGATCCGGCTGCTAACAA |
| | | vector | | | | AG |
| | | backbone | | | | |
| | | for MGC | | | | |
| | | entry | | | | |
| | | plasmid | | | | |
| primer | 77 | Amplify | nucleo- | artificial | | CTCCTTGCATGCTCTAGAATTAATTA |
| | | SV40 NLS- | tide | sequence | | ACCATTCCTTGCGGCGGCG |
| | | vector | | | | |
| | | backbone | | | | |
| | | for MGC | | | | |
| | | entry | | | | |
| | | plasmid | | | | |
| primer | 78 | Amplify | nucleo- | artificial | | AGGAATGGTTAATTAATTCTAGAGCA |
| | | vector | tide | sequence | | TGCAAGGAGATGGCGCCCAACA |
| | | backbone | | | | |
| | | for MGC | | | | |
| | | entry | | | | |
| | | plasmid | | | | |
| primer | 79 | Amplify | nucleo- | artificial | | GTGATGATGATGACTGCTGCCCATGG |
| | | vector | tide | sequence | | TATATCTCCTTCTTAAAGTTAAAC |
| | | backbone | | | | |
| | | for MGC | | | | |
| | | entry | | | | |
| | | plasmid | | | | |
| primer | 80 | Amplify | nucleo- | artificial | | GAAAGTAGTGGAGGCAGCTCTGGTGG |
| | | nMG4-2 | tide | sequence | | GTCCATGCTTCGTGAGCCCGGT |
| | | (D28A) for | | | | |
| | | pMGA | | | | |
| | | expression | | | | |
| | | plasmid | | | | |
| primer | 81 | Amplify | nucleo- | artificial | | CCTCTTCTTCTTGGGAGACCCGCCAG |
| | | nMG4-2 | tide | sequence | | AGCCATCGTTAGCATAACGAATTTCC |
| | | (D28A) for | | | | C |
| | | pMGA | | | | |
| | | expression | | | | |
| | | plasmid | | | | |
| primer | 82 | Amplify | nucleo- | artificial | | GAAAGTAGTGGAGGCAGCTCTGGTGG |
| | | nMG7-1 | tide | sequence | | GTCCATGAGTAATAAAACAATTTTAG |
| | | (D10A) for | | | | GCCTG |
| | | pMGA | | | | |
| | | expression | | | | |
| | | plasmid | | | | |
| primer | 83 | Amplify | nucleo- | artificial | | CCTCTTCTTCTTGGGAGACCCGCCAG |
| | | nMG7-1 | tide | sequence | | AGCCTTTGGGCAAGCCTAAAACATC |
| | | (D10A) for | | | | |
| | | pMGA | | | | |
| | | expression | | | | |
| | | plasmid | | | | |
| primer | 84 | Amplify | nucleo- | artificial | | GAAAGTAGTGGAGGCAGCTCTGGTGG |
| | | nMG16-1 | tide | sequence | | GTCCATGATTAAGAATATCTTGGGTC |
| | | (D9A) for | | | | TG |
| | | pMGA | | | | |
| | | expression | | | | |
| | | plasmid | | | | |
| primer | 85 | Amplify | nucleo- | artificial | | CCTCTTCTTCTTGGGAGACCCGCCAG |
| | | nMG16-1 | tide | sequence | | AGCCCGCTTTGGTGATGCGGCC |
| | | (D9A) for | | | | |
| | | pMGA | | | | |
| | | expression | | | | |
| | | plasmid | | | | |
| primer | 86 | Amplify | nucleo- | artificial | | ACATCCGAAAGTGCGACACCTGAATC |
| | | nMG4-2 | tide | sequence | | TGCCATGCTTCGTGAGCCCGGT |
| | | (D28A) for | | | | |
| | | pMGC | | | | |
| | | expression | | | | |
| | | plasmid | | | | |
| primer | 87 | Amplify | nucleo- | artificial | | ATCTGAAAGATTGGTAGAACCACCAG |
| | | nMG4-2 | tide | sequence | | ACCCATCGTTAGCATAACGAATTTCC |
| | | (D28A) for | | | | C |
| | | pMGC | | | | |
| | | expression | | | | |
| | | plasmid | | | | |
| primer | 88 | Amplify | nucleo- | artificial | | ACATCCGAAAGTGCGACACCTGAATC |
| | | nMG7-1 | tide | sequence | | TGCCATGAGTAATAAAACAATTTTAG |
| | | (D10A) for | | | | GCCT |
| | | pMGC | | | | |
| | | expression | | | | |
| | | plasmid | | | | |
| primer | 89 | Amplify | nucleo- | artificial | | ATCTGAAAGATTGGTAGAACCACCAG |
| | | nMG7-1 | tide | sequence | | ACCCTTTGGGCAAGCCTAAAACATC |
| | | (D10A) for | | | | |
| | | pMGC | | | | |
| | | expression | | | | |
| | | plasmid | | | | |
| primer | 90 | Amplify | nucleo- | artificial | | ACATCCGAAAGTGCGACACCTGAATC |
| | | nMG16-1 | tide | sequence | | TGCCATGATTAAGAATATCTTGGGTC |
| | | (D9A) for | | | | TG |
| | | pMGC | | | | |
| | | expression | | | | |
| | | plasmid | | | | |
| primer | 91 | Amplify | nucleo- | artificial | | ATCTGAAAGATTGGTAGAACCACCAG |
| | | nMG16-1 | tide | sequence | | ACCCCGCTTTGGTGATGCGGCC |
| | | (D9A) for | | | | |
| | | pMGC | | | | |
| | | expression | | | | |
| | | plasmid | | | | |
| primer | 92 | Amplify | nucleo- | artificial | | CACAGCCCGTTTTCATCATATTTAAT |
| | | MGA4- | tide | sequence | | CACTATAGTGAGTCGTATTAAGGT |
| | | 2_sgRNA | | | | |
| | | spacer 1 | | | | |
| primer | 93 | Amplify | nucleo- | artificial | | TCACTATAGTGATTAAATATGATGAA |
| | | MGA4- | tide | sequence | | AACGGGCTGTGGTTTGATGGGTTC |
| | | 2_sgRNA | | | | |
| | | spacer 1 | | | | |
| primer | 94 | Amplify | nucleo- | artificial | | CACAGCCTTCACTTACGCCAATGTCG |
| | | MGA4- | tide | sequence | | TTCTATAGTGAGTCGTATTAAGGT |
| | | 2_sgRNA | | | | |
| | | spacer 2 | | | | |
| primer | 95 | Amplify | nucleo- | artificial | | TCACTATAGAACGACATTGGCGTAAG |
| | | MGA4- | tide | sequence | | TGAAGGCTGTGGTTTGATGGGTTC |
| | | 2_sgRNA | | | | |
| | | spacer 2 | | | | |
| primer | 96 | Amplify | nucleo- | artificial | | CACAGCGGCCATAATTCAATTCGCGC |
| | | MGA4- | tide | sequence | | GTCTATAGTGAGTCGTATTAAGGT |
| | | 2_sgRNA | | | | |
| | | spacer 3 | | | | |
| primer | 97 | Amplify | nucleo- | artificial | | TCACTATAGACGCGCGAATTGAATTA |
| | | MGA4- | tide | sequence | | TGGCCGCTGTGGTTTGATGGGTTC |
| | | 2_sgRNA | | | | |
| | | spacer 3 | | | | |
| primer | 98 | Amplify | nucleo- | artificial | | CACAACTCCGCCACATATCCTGATCT |
| | | MGA7- | tide | sequence | | TCCTATAGTGAGTCGTATTAAGGT |
| | | 1_sgRNA | | | | |
| | | spacer 1 | | | | |
| primer | 99 | Amplify | nucleo- | artificial | | CTATAGGAAGATCAGGATATGTGGCG |
| | | MGA7- | tide | sequence | | GAGTTGTGAATGGCTTTCAGAAAT |
| | | 1_sgRNA | | | | |
| | | spacer 1 | | | | |
| primer | 100 | Amplify | nucleo- | artificial | | CACAACACAGTTTCGGGTTTTCGACG |
| | | MGA7- | tide | sequence | | TTCTATAGTGAGTCGTATTAAGGT |
| | | 1_sgRNA | | | | |
| | | spacer 2 | | | | |
| primer | 101 | Amplify | nucleo- | artificial | | TATAGAACGTCGAAAACCCGAAACTG |
| | | MGA7- | tide | sequence | | TGTTGTGAATGGCTTTCAGAAATG |
| | | 1_sgRNA | | | | |
| | | spacer 2 | | | | |
| primer | 102 | Amplify | nucleo- | artificial | | CACAACATTGGCACCATGCCGTGGGT |
| | | MGA7- | tide | sequence | | TTCTATAGTGAGTCGTATTAAGGT |
| | | 1_sgRNA | | | | |
| | | spacer 3 | | | | |
| primer | 103 | Amplify | nucleo- | artificial | | TATAGAAACCCACGGCATGGTGCCAA |
| | | MGA7- | tide | sequence | | TGTTGTGAATGGCTTTCAGAAATG |
| | | 1_sgRNA | | | | |
| | | spacer 3 | | | | |
| primer | 104 | Amplify | nucleo- | artificial | | CACAACCTTCGCTATTACGCCAGCTG |
| | | MGA16- | tide | sequence | | GCCTATAGTGAGTCGTATTAAGGT |
| | | 1_sgRNA | | | | |
| | | spacer 1 | | | | |
| primer | 105 | Amplify | nucleo- | artificial | | TATAGGCCAGCTGGCGTAATAGCGAA |
| | | MGA16- | tide | sequence | | GGTTGTGTATGGAAACATACACAA |
| | | 1_sgRNA | | | | |
| | | spacer 1 | | | | |
| primer | 106 | Amplify | nucleo- | artificial | | CACAACCTGGCGACCTGCGTTTCACC |
| | | MGA16- | tide | sequence | | CTCTATAGTGAGTCGTATTAAGGT |
| | | 1_sgRNA | | | | |
| | | spacer 2 | | | | |
| primer | 107 | Amplify | nucleo- | artificial | | TATAGAGGGTGAAACGCAGGTCGCCA |
| | | MGA16- | tide | sequence | | GGTTGTGTATGGAAACATACACAA |
| | | 1_sgRNA | | | | |
| | | spacer 2 | | | | |
| primer | 108 | Amplify | nucleo- | artificial | | CACAACGGTTTTCGACGTTCAGACGT |
| | | MGA16- | tide | sequence | | AGCTATAGTGAGTCGTATTAAGGT |
| | | 1_sgRNA | | | | |
| | | spacer 3 | | | | |
| primer | 109 | Amplify | nucleo- | artificial | | ATAGCTACGTCTGAACGTCGAAAACC |
| | | MGA16- | tide | sequence | | GTTGTGTATGGAAACATACACAA |
| | | 1_sgRNA | | | | |
| | | spacer 3 | | | | |
| primer | 110 | Amplify | nucleo- | artificial | | ATCCACATCTGTGAAAGAAACTATAG |
| | | ABE8.17m | tide | sequence | | TGAGTCGTATTAGGA |
| | | sgRNA | | | | |
| | | spacer 1 | | | | |
| primer | 111 | Amplify | nucleo- | artificial | | TTTCTTTCACAGATGTGGATGTTTTA |
| | | ABE8.17m | tide | sequence | | GAGCTAGAAATAGCAAGTT |
| | | sgRNA | | | | |
| | | spacer 1 | | | | |
| primer | 112 | Amplify | nucleo- | artificial | | CTCATCCGCCACATATCCTGCTATAG |
| | | ABE8.17m | tide | sequence | | TGAGTCGTATTAGGA |
| | | sgRNA | | | | |
| | | spacer 2 | | | | |
| primer | 113 | Amplify | nucleo- | artificial | | CAGGATATGTGGCGGATGAGGTTTTA |
| | | ABE8.17m | tide | sequence | | GAGCTAGAAATAGCAAGTT |
| | | sgRNA | | | | |
| | | spacer 2 | | | | |
| primer | 114 | Amplify | nucleo- | artificial | | ATCGCGTGGGCGTATTCGCACTATAG |
| | | ABE8.17m | tide | sequence | | TGAGTCGTATTAGGA |
| | | sgRNA | | | | |
| | | spacer 3 | | | | |
| primer | 115 | Amplify | nucleo- | artificial | | TGCGAATACGCCCACGCGATGTTTTA |
| | | ABE8.17m | tide | sequence | | GAGCTAGAAATAGCAAGTT |
| | | sgRNA | | | | |
| | | spacer 3 | | | | |
| primer | 116 | Amplify | nucleo- | artificial | | CACAGCGATAATGCGAACAGCGCACG |
| | | MGC4- | tide | sequence | | GCCTATAGTGAGTCGTATTAAGGT |
| | | 2_spacer 1 | | | | |
| primer | 117 | Amplify | nucleo- | artificial | | CACTATAGGCCGTGCGCTGTTCGCAT |
| | | MGC4- | tide | sequence | | TATCGCTGTGGTTTGATGGGTTC |
| | | 2_spacer 1 | | | | |
| primer | 118 | Amplify | nucleo- | artificial | | CACAGCCTGACGAAACGCCTGCCAGT |
| | | MGC4- | tide | sequence | | ATCTATAGTGAGTCGTATTAAGGT |
| | | 2_spacer 2 | | | | |
| primer | 119 | Amplify | nucleo- | artificial | | CACTATAGATACTGGCAGGCGTTTCG |
| | | MGC4- | tide | sequence | | TCAGGCTGTGGTTTGATGGGTTC |
| | | 2_spacer 2 | | | | |
| primer | 120 | Amplify | nucleo- | artificial | | CACAGCGCGTGGGACGCGGCGGGGAG |
| | | MGC4- | tide | sequence | | TGCTATAGTGAGTCGTATTAAGGT |
| | | 2_spacer 3 | | | | |
| primer | 121 | Amplify | nucleo- | artificial | | CACTATAGCACTCCCCGCCGCGTCCC |
| | | MGC4- | tide | sequence | | ACGCGCTGTGGTTTGATGGGTTC |
| | | 2_spacer 3 | | | | |
| primer | 122 | Amplify | nucleo- | artificial | | CACAACGACGTAGTGTGACGCGATCG |
| | | MGC7- | tide | sequence | | GCCTATAGTGAGTCGTATTAAGGT |
| | | 1_spacer 1 | | | | |
| primer | 123 | Amplify | nucleo- | artificial | | TATAGGCCGATCGCGTCACACTACGT |
| | | MGC7- | tide | sequence | | CGTTGTGAATGGCTTTCAGAAATG |
| | | 1_spacer 1 | | | | |
| primer | 124 | Amplify | nucleo- | artificial | | CACAACTCGCCGGTAGCCAGCGCGGA |
| | | MGC7- | tide | sequence | | TCCTATAGTGAGTCGTATTAAGGT |
| | | 1_spacer 2 | | | | |
| primer | 125 | Amplify | nucleo- | artificial | | TATAGGATCCGCGCTGGCTACCGGCG |
| | | MGC7- | tide | sequence | | AGTTGTGAATGGCTTTCAGAAATG |
| | | 1_spacer 2 | | | | |
| primer | 126 | Amplify | nucleo- | artificial | | CACAACCATACTGCACCGGGCGGGAA |
| | | MGC7- | tide | sequence | | GGCTATAGTGAGTCGTATTAAGGT |
| | | 1_spacer 3 | | | | |
| primer | 127 | Amplify | nucleo- | artificial | | TATAGCCTTCCCGCCCGGTGCAGTAT |
| | | MGC7- | tide | sequence | | GGTTGTGAATGGCTTTCAGAAATG |
| | | 1_spacer 3 | | | | |
| primer | 128 | Amplify | nucleo- | artificial | | CACAACACCGTAATGGGATAGGTCAC |
| | | MGC16- | tide | sequence | | GTCTATAGTGAGTCGTATTAAGGT |
| | | 1_spacer 1 | | | | |
| primer | 129 | Amplify | nucleo- | artificial | | TATAGACGTGACCTATCCCATTACGG |
| | | MGC16- | tide | sequence | | TGTTGTGTATGGAAACATACACAA |
| | | 1_spacer 1 | | | | |
| primer | 130 | Amplify | nucleo- | artificial | | CACAACTCCGTGGGAACAAACGGCGG |
| | | MGC16- | tide | sequence | | ATCTATAGTGAGTCGTATTAAGGT |
| | | 1_spacer 2 | | | | |
| primer | 131 | Amplify | nucleo- | artificial | | ATAGATCCGCCGTTTGTTCCCACGGA |
| | | MGC16- | tide | sequence | | GTTGTGTATGGAAACATACACAAT |
| | | 1_spacer 2 | | | | |
| primer | 132 | Amplify | nucleo- | artificial | | CACAACTGCTTCAATCAGCGTGCCGT |
| | | MGC16- | tide | sequence | | CGCTATAGTGAGTCGTATTAAGGT |
| | | 1_spacer 3 | | | | |
| primer | 133 | Amplify | nucleo- | artificial | | ATAGCGACGGCACGCTGATTGAAGCA |
| | | MGC16- | tide | sequence | | GTTGTGTATGGAAACATACACAAT |
| | | 1_spacer 3 | | | | |
| primer | 134 | Amplify | nucleo- | artificial | | TGTGGAGCGACATCCAGAGGCTATAG |
| | | BE3 sgRN | tide | sequence | | TGAGTCGTATTAGGA |
| | | A spacer 1 | | | | |
| primer | 135 | Amplify | nucleo- | artificial | | CCTCTGGATGTCGCTCCACAGTTTTA |
| | | BE3 sgRN | tide | sequence | | GAGCTAGAAATAGCAAGTT |
| | | A spacer 1 | | | | |
| primer | 136 | Amplify | nucleo- | artificial | | TAATTCAATTCGCGCGTCCCCTATAG |
| | | BE3 sgRN | tide | sequence | | TGAGTCGTATTAGGA |
| | | A spacer 2 | | | | |
| primer | 137 | Amplify | nucleo- | artificial | | GGGACGCGCGAATTGAATTAGTTTTA |
| | | BE3 sgRN | tide | sequence | | GAGCTAGAAATAGCAAGTT |
| | | A spacer 2 | | | | |
| primer | 138 | Amplify | nucleo- | artificial | | AGTCACGACGTTGTAAAACGCTATAG |
| | | BE3 sgRN | tide | sequence | | TGAGTCGTATTAGGA |
| | | A spacer 3 | | | | |
| primer | 139 | Amplify | nucleo- | artificial | | CGTTTTACAACGTCGTGACTGTTTTA |
| | | BE3 sgRN | tide | sequence | | GAGCTAGAAATAGCAAGTT |
| | | A spacer 3 | | | | |
| primer | 140 | For lacZ | nucleo- | artificial | | CGAACATCCAAAAGTTTGTGTTTTT |
| | | sequencing | tide | sequence | | |
| primer | 141 | For lacZ | nucleo- | artificial | | TGAGCGCATTTTTACGCGC |
| | | sequencing | tide | sequence | | |
| primer | 142 | For lacZ | nucleo- | artificial | | GAAAACGGCAACCCGTGG |
| | | sequencing | tide | sequence | | |
| primer | 143 | Amplify | nucleo- | artificial | | CCGCCGCCGCAAGGAATGGTTTAATT |
| | | sgRNA | tide | sequence | | AATTACGGCCAGTCATGCATAATC |
| | | expression | | | | |
| | | cassette | | | | |
| primer | 144 | Amplify | nucleo- | artificial | | GACTGTTGGGCGCCATCTCCTTGCAT |
| | | sgRNA | tide | sequence | | GCTCACTGAGCCTCCACCTAGCCT |
| | | expression | | | | |
| | | cassette | | | | |
| PAM | 145 | nMG4-2 | nucleo- | artificial | | YRnMCC |
| | | (D28A) | tide | sequence | | |
| | | nickase | | | | |
| | | PAM | | | | |
| PAM | 146 | nMG7-1 | nucleo- | artificial | | nRRnCg |
| | | (D10A) | tide | sequence | | |
| | | nickase | | | | |
| | | PAM | | | | |
| PAM | 147 | nMG16-1 | nucleo- | artificial | | nRRnMC |
| | | (D9A) | tide | sequence | | |
| | | nickase | | | | |
| | | PAM | | | | |
| NLS | 148 | SV40 | nucleo- | artificial | Nuclear | PKKKRKV |
| | | | tide | sequence | localization | |
| | | | | | sequence | |
| NLS | 149 | nucleoplasm | nucleo- | | Nuclear | KRPAATKKAGQAKKKK |
| | | in bipartite | tide | | localization | |
| | | NLS | | | sequence | |
| NLS | 150 | c-myc NLS | nucleo- | | Nuclear | PAAKRVKLD |
| | | | tide | | localization | |
| | | | | | sequence | |
| NLS | 151 | c-myc NLS | nucleo- | | Nuclear | RQRRNELKRSP |
| | | | tide | | localization | |
| | | | | | sequence | |
| NLS | 152 | hRNPA1 | nucleo- | | Nuclear | NQSSNFGPMKGGNFGGRSSGPYG |
| | | M9 NLS | tide | | localization | GGGQYFAKPRNQGGY |
| | | | | | sequence | |
| NLS | 153 | Importin- | nucleo- | | Nuclear | RMRIZFKNKGKDTAELRRRRVEV |
| | | alpha IBB | tide | | localization | SVELRKAKKDEQILKRRNV |
| | | domain | | | sequence | |
| NLS | 154 | Myoma T | nucleo- | | Nuclear | VSRKRPRP |
| | | protein | tide | | localization | |
| | | | | | sequence | |
| NLS | 155 | Myoma T | nucleo- | | Nuclear | PPKKARED |
| | | protein | tide | | localization | |
| | | | | | sequence | |
| NLS | 156 | p53 | nucleo- | | Nuclear | PQPKKKPL |
| | | | tide | | localization | |
| | | | | | sequence | |
| NLS | 157 | mouse c-abl | nucleo- | | Nuclear | SALIKKKKKMAP |
| | | IV | tide | | localization | |
| | | | | | sequence | |
| NLS | 158 | influenza | nucleo- | | Nuclear | DRLRR |
| | | virus NS1 | tide | | localization | |
| | | | | | sequence | |
| NLS | 159 | influenza | nucleo- | | Nuclear | PKQKKRK |
| | | virus NS1 | tide | | localization | |
| | | | | | sequence | |
| NLS | 160 | Hepatitis | nucleo- | | Nuclear | RKLKKKIKKL |
| | | virus delta | tide | | localization | |
| | | antigen | | | sequence | |
| NLS | 161 | mouse Mx1 | nucleo- | | Nuclear | REKKKFLKRR |
| | | protein | tide | | localization | |
| | | | | | sequence | |
| NLS | 162 | human | nucleo- | | Nuclear | KRKGDEVDGVDEVAKKKSKK |
| | | poly(ADP- | tide | | localization | |
| | | ribose) | | | sequence | |
| | | polymerase | | | | |
| NLS | 163 | steroid | nucleo- | | Nuclear | RKCLQAGMNLEARKTKK |
| | | hormone | tide | | localization | |
| | | receptor | | | sequence | |
| | | (human) | | | | |
| | | glucocorticoid | | | | |
| Adenosine | 164 | TadA* | protein | unknown | unknown | MSEVEFSHEYWMRHALTLAKRAR |
| Deaminase | | (ABE8.17m) | | | | DEREVPVGAVLVLNNRVIGEGWN |
| | | | | | | RAIGLHDPTAHAEIMALRQGGLV |
| | | | | | | MQNYRLIDATLYSTFEPCVMCAG |
| | | | | | | AMIHSRIGRVVFGVRNAKTGAAG |
| | | | | | | SLMDVLHYPGMNHRVEITEGILAD |
| | | | | | | ECAALLCYFFRMPRRVFNAQKKA |
| | | | | | | QSSTD |

### EXAMPLES

### Example 1.—Plasmid Construction for Base Editors

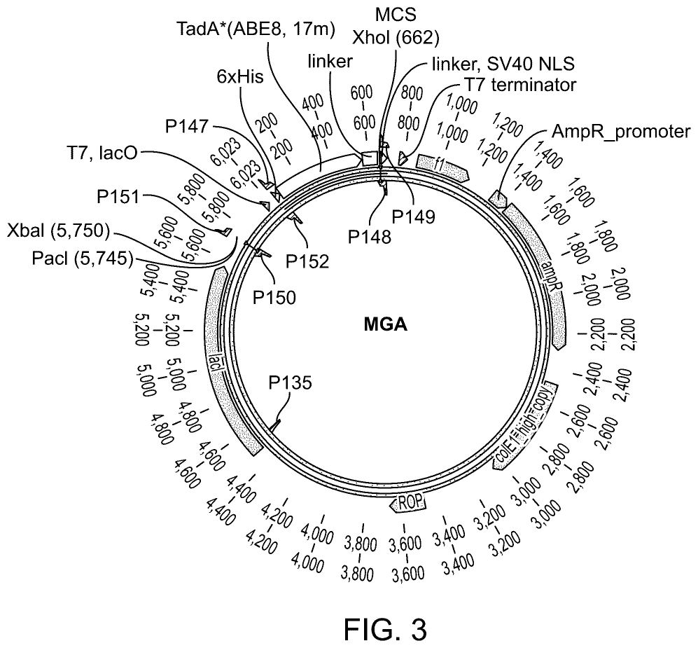

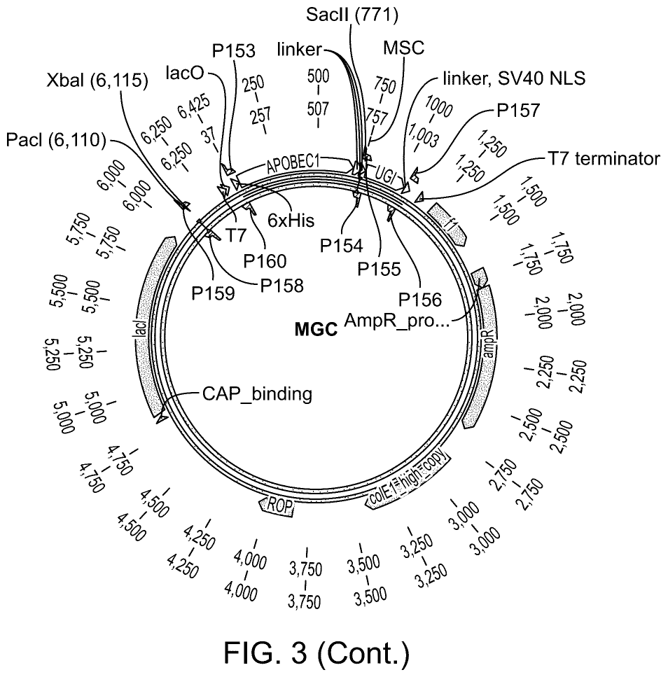

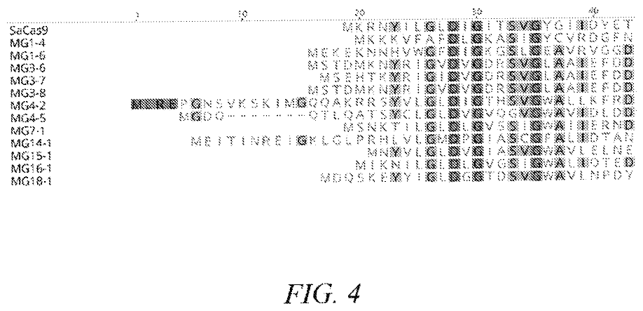

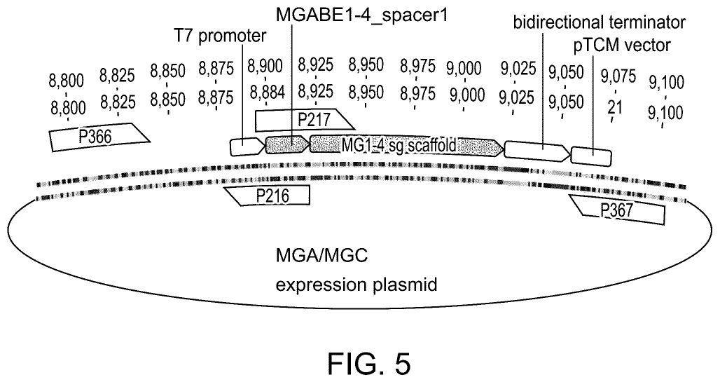

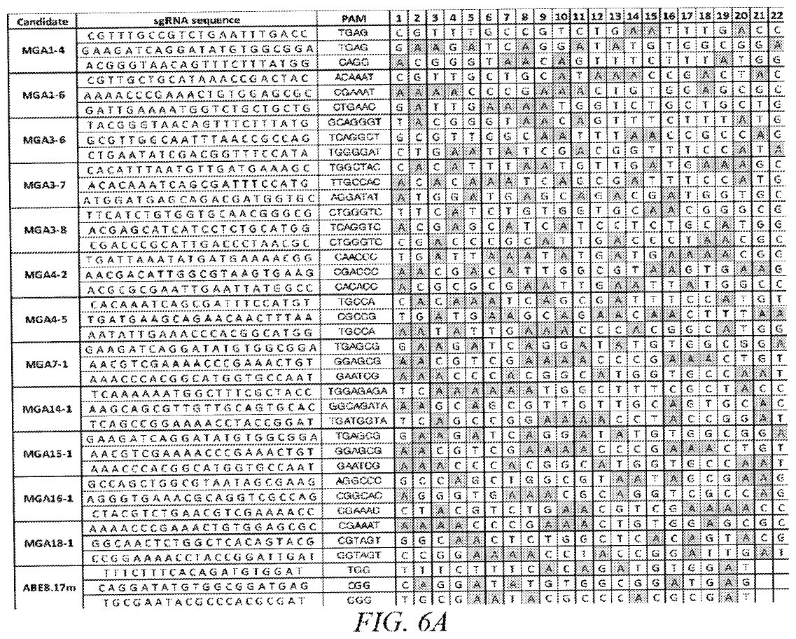

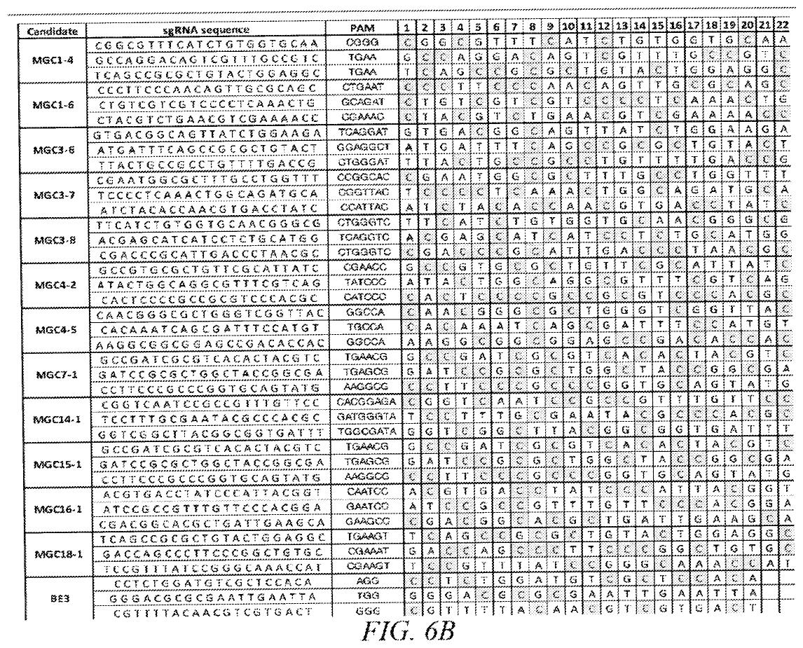

To construct the MGA (Metagenomi adenine base editor) entry plasmid containing T7 promoter-His tag-TadA*(ABE8.17m)-SV40 NLS, three pieces of DNA fragments were amplified from pAL6. To construct the MGC (Metagenomi cytosine base editor) entry plasmid containing T7 promoter-His tag-APOBEC1(BE3)-UGI-SV40 NLS, APOBEC1 and UGI-SV40 NLS were amplified from pAL9 and two pieces of vector backbones were amplified from pAL6 (see FIG. 3).

To introduce mutations to the effectors, pMGA or pMGC plasmids containing effector gene sequences were amplified by Q5 DNA polymerase with forward primers incorporating desirable mutations and reverse primers. The linear DNA fragments were then phosphorylated, ligated, and the DNA templates were digested by DpnI using KLD Enzyme Mix (New England Biolabs) per manufacturer's instructions.

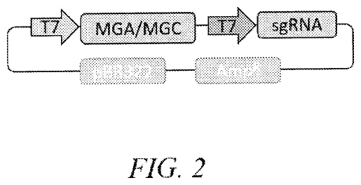

To generate pMGA and pMGC expression plasmids, genes were amplified from plasmids carrying mutated effectors and cloned into MGA and MGC entry plasmids via XhoI and SacII sites, respectively. To clone sgRNA expression cassettes comprising T7 promoter-sgRNA-bidirectional terminator into BE expression plasmids, one set of primers (P366 as the forward primer) was used to amplify a T7 promoter-spacer sequence while another set of primers (P367 as the reverse primer) was used to amplify spacer sequence-sgRNA scaffold-bidirectional terminator, in which pTCM plasmids were used as templates (see FIG. 2). The two fragments were assembled into pMGA and pMGC via XbaI sites, resulting pMGA-sgRNA and pMGC-sgRNA, respectively.

TABLE 3
Summary of constructs made for screening
systems described herein

| # | Application | Candidate |
|---|---|---|
| 1 | ABE | MGA4-2-sgRNA1 |
| 2 | | MGA4-2-sgRNA2 |
| 3 | | MGA4-2-sgRNA3 |
| 4 | | MGA7-1-sgRNA1 |
| 5 | | MGA7-1-sgRNA2 |
| 6 | | MGA7-1-sgRNA3 |
| 7 | | MGA16-1-sgRNA1 |
| 8 | | MGA16-1-sgRNA2 |
| 9 | | MGA16-1-sgRNA3 |
| 10 | | ABE8.17m-sgRNA1 |
| 11 | | ABE8.17m-sgRNA2 |
| 12 | | ABE8.17m-sgRNA3 |
| 13 | CBE | MGC4-2-sgRNA1 |
| 14 | | MGC4-2-sgRNA2 |
| 15 | | MGC4-2-sgRNA3 |
| 16 | | MGC7-1-sgRNA1 |
| 17 | | MGC7-1-sgRNA2 |
| 18 | | MGC7-1-sgRNA3 |
| 19 | | MGC16-1-sgRNA1 |
| 20 | | MGC16-1-sgRNA2 |
| 21 | | MGC16-1-sgRNA3 |
| 22 | | BE3-sgRNA1 |
| 23 | | BE3-sgRNA2 |
| 24 | | BE3-sgRNA3 |

All amplified DNA fragments were purified by QIAquick Gel Extraction Kit (Qiagen), assembled via NEBuilder HiFi DNA Assembly (New England Biolabs), and the resulting assemblies were propagated via Endura Electrocompetent cells (Lucergen) per manufacturer's instructions. DNA sequences of all cloned genes were confirmed at ELIM BIOPHARM.

TABLE 4
Conserved catalytic residues parsed out
for selected systems described herein

| Nickase Candidate | Length |
|---|---|
| nMG4-2 (D28A) (SEQ ID | 1043 |
| NO: 21) | |
| nMG7-1 (D10A) (SEQ ID | 1168 |
| NO: 22) | |
| nMG16-1 (D9A) (SEQ ID | 1154 |
| NO: 23) | |

### Example 2.—Protein Expression and Purification

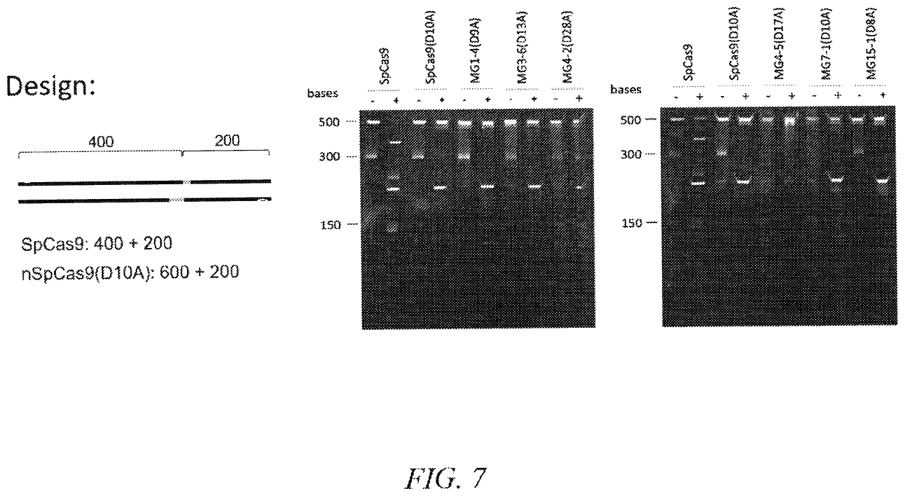

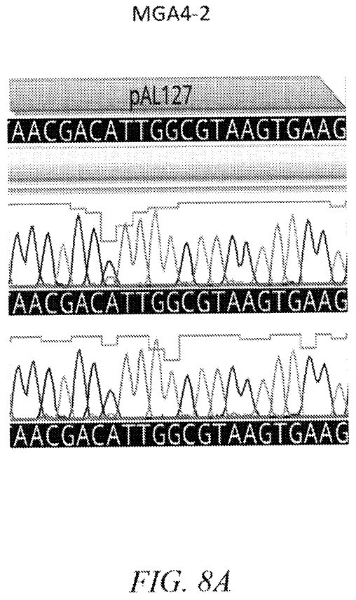

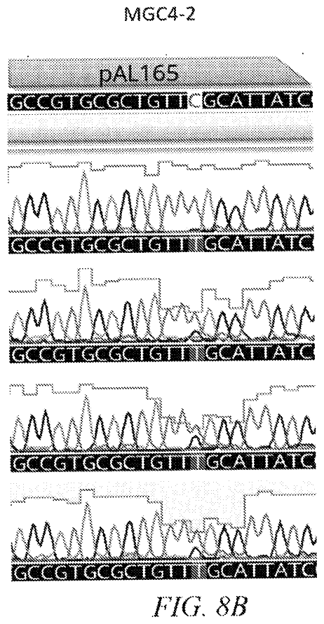

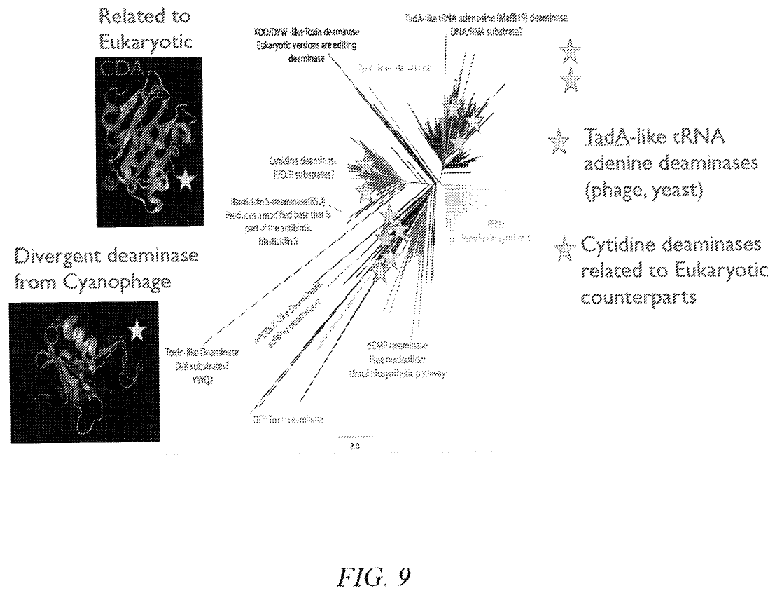

The T7 promoter driven mutated effector genes in the pMGX plasmid were expressed in E. coli BL21 (DE3) cells in Magic Media per manufacturer's instructions (Thermo). After 40 hours 16° C. incubation the cells were harvested, suspended in lysis buffer (HisTrap equilibration buffer: 20 mM Tris (Sigma T2319-100 ML), 300 mM sodium chloride (VWR VWRVE529-500 ML), 5% glycerol, 10 mM MgCl₂, with 10 mM imidazole (Sigma 68268-100 ML-F); pH 7.5) and EDTA-free protease inhibitor (Pierce), and frozen in the −80° C. freezer. The cells were then thawed on ice, sonicated, clarified, and filtered before affinity purification. The protein was applied to Cytiva 5 ml HisTrap FF column on the Akta Avant FPLC per the manufacturer's specifications and the protein was eluted in an isocratic elution of 20 mM Tris (Sigma T2319-100 ML), 300 mM sodium chloride (VWR VWRVE529-500 ML), 5% glycerol, 10 mM MgCl₂, with 250 mM imidazole (Sigma 68268-100 ML-F); pH 7.5. Eluted fractions containing the His-tagged effector proteins were concentrated and buffer exchanged into 50 mM Tris-HCl, 300 mM NaCl, 1 mM TCEP, 5% glycerol; pH 7.5. The protein concentration was determined by bicinchoninic acid assay (Thermo) and adjusted after determining the relative purity by SDS PAGE densitometry in Image Lab (Bio-Rad) (see FIG. 7).

### Example 3.—In Vitro Nickase Assay

6-carboxyfluorescein (6-FAM) labeled primers P141 and P146 synthesized by IDT were used to amplify linear fragments containing targeting sequences of effectors from pMGK plasmids by Q5 DNA polymerase. DNA fragments containing T7 promoter followed by 20- or 22-bp spacer sequences were transcribed in vitro using HiScribe T7 High Yield RNA Synthesis Kit (New England Biolabs) per manufacturer's instructions. The synthesized sgRNAs with sequences listed in Table 2 were purified by Monarch RNA Cleanup Kit (New England Biolabs) according to the user manual and concentrations were measured by Nanodrop.

To determine nickase activity, the purified mutated effector was first supplemented with its cognate sgRNA, and the reaction was initiated by adding the linear DNA substrate in a 15 L reaction mixture containing 10 mM Tris pH 7.5, 10 mM MgCl₂, and 100 mM NaCl, 150 nM enzyme, 150 nM RNA, and 15 nM DNA. The reaction was incubated at 37° C. for 2 h, and digested DNA was purified by AMPure XP SPRI paramagnetic beads (Beckman Coulter) and eluted with 6 μL TE buffer (10 mM Tris, 1 mM EDTA; pH 8.0). The nicked DNA was resolved on a 10% TBE-Urea denaturing gel (Biorad) and imaged by ChemiDoc (Bio-Rad) (see FIG. 7).

### Example 4.—Base Editor Screening and Editing Efficiency in E. coli

Plasmids were transformed into Lucergen's electrocompetent BL21(DE3) cells according to manufacturer's instructions. After electroporation, cells were recovered with expression recovery media at 37° C. for 1 h and spread on LB plates containing 100 L/mg ampicillin and 0.1 mM IPTG. After overnight growth at 37° C., colonies were picked and lacZ gene was amplified by Q5 DNA polymerase (New England Biolabs) with primers P137 and P360. The resulting PCR products were purified and sequenced by Sanger sequencing at ELIM BIOPHARM. Base edits were determined by examining whether there exists C to T conversion or A to G conversion in the targeted protospacer regions for cytosine base editors or adenine base editors, respectively.

To evaluate editing efficiency in E. coli, plasmids were transformed into electrocompetent BL21(DE3) (Lucergen) and the electroporated cells were recovered with expression recovery media at 37° C. for 1 h. 10 μL of recovered cells were then inoculated into 990 μL SOB containing 100 μL/mg ampicillin and 0.1 mM IPTG in a 96-well deep well plate, and grown at 37° C. for 20 h. 1 μL cells induced for base editor expression were used for amplification of lacZ gene in a 20 μL PCR reaction (Q5 DNA polymerase) with primers P137 and P360. The resulting PCR products were purified and sequenced by Sanger sequencing at ELIM BIOPHARM. Quantification of editing efficiency was processed by Edit R.

TABLE 5
MG base editors described herein with
associated PAM and deaminases

| Candidate | Type | PAM | Deaminase | Nickase |
|---|---|---|---|---|
| MGA4-2 | II | YRnMCC | TadA* (ABE8.17m) | nMG4-2 (D28A) |
| | | (SEQ ID | (SEQ ID NO: 164) | (SEQ ID NO: 21) |
| | | NO: 145) | | |

### Example 5.—Protein Nucleofection and Amplicon Seq (Prophetic)

Nucleofection is conducted as per the manufacturer's recommendations using a Lonza 4D nucleofector and the Lonza SF Cell Line 4D-Nucleofector X Kit S (cat. no. V4XC-2032). After formulating the SF nucleofection buffer, 200,000 cells are resuspended in 5 μl of buffer per nucleofection. In the remaining 15 μl of buffer per nucleofection, 20 pmol of chemically modified sgRNA from Synthego is combined with 18 pmol of ABE8e protein and incubated for 5 min at room temperature to complex. Cells are added to the 20 μl nucleofection cuvettes, followed by protein solution, and the mixture is triturated to mix. Cells are nucleofected with program CM-130, immediately after which 80 μl of warmed media is added to each well for recovery. After 5 min, 25 μl from each sample is added to 250 μl of fresh media in a 48-well poly-d-lysine plate (Corning). Cells are then treated in the same way as lipofected cells above for genomic DNA extraction after three more days of culture.

Following Illumina barcoding, PCR products are pooled and purified by electrophoresis with a 2% agarose gel using a Monarch DNA Gel Extraction Kit (New England Biolabs), eluting with 30 μl H2O. DNA concentration is quantified with a Qubit dsDNA High Sensitivity Assay Kit (Thermo Fisher Scientific) and sequenced on an Illumina MiSeq instrument (paired-end read, R1: 250-280 cycles, R2: 0 cycles) according to the manufacturer's protocols.

Sequencing reads are demultiplexed using the MiSeq Reporter (Illumina) and FASTQ files are analyzed using CRISPResso2. Dual editing in individual alleles are analyzed by a Python script. Base editing values are representative of n=3 independent biological replicates collected by different researchers, with the mean±s.d. shown. Base editing values are reported as a percentage of the number of reads with adenine mutagenesis over the total aligned reads.

### Example 6.—Plasmid Nucleofection and Whole Genome Seq (Prophetic)

PCR is performed using Phusion U Green Multiplex PCR Master Mix (ThermoFisher Scientific). All plasmids are assembled by the uracil-specific excision reagent (USER) cloning method. Guide RNA plasmids for SpCas9, SaCas9 and all engineered variants are assembled. Plasmids for mammalian cell transfections are prepared using the ZymoPURE Plasmid Midiprep kit (Zymo Research Corporation). HEK293T cells (ATCC CRL-3216) are cultured in Dulbecco's modified Eagle's medium (Corning) supplemented with 10% fetal bovine serum (ThermoFisher Scientific) and maintained at 37° C. with 5% CO2.

HEK293T cells are seeded on 48-well poly-d-lysine plates (Corning) in the same culture medium. Cells are transfected 12-16 h after plating with 1.5 μl Lipofectamine 2000 (ThermoFisher Scientific) using 750 ng base editor plasmid, 250 ng guide RNA plasmid and 10 ng green fluorescent protein as a transfection control. Cells are cultured for 3 d with media exchanged following the first day, then washed with Å˜1 PBS (ThermoFisher Scientific), followed by genomic DNA extraction by addition of 100 μl freshly prepared lysis buffer (10 mM Tris-HCl, pH 7.5, 0.05% SDS, 25 μg ml−1 proteinase K (ThermoFisher Scientific)) directly into each transfected well. The mixture is incubated at 37° C. for 1 h then heat inactivated at 80° C. for 30 min. Genomic DNA lysate is subsequently used immediately for high-throughput sequencing (HTS).

HTS of genomic DNA from HEK293T cells is performed. Following Illumina barcoding, PCR products are pooled and purified by electrophoresis with a 2% agarose gel using a Monarch DNA Gel Extraction Kit (NEB), eluting with 30 μl H2O. DNA concentration is quantified with Qubit dsDNA High Sensitivity Assay Kit (ThermoFisher Scientific) and sequenced on an Illumina MiSeq instrument (paired end read, R1: 250-280 cycles, R2: 0 cycles) according to the manufacturer's protocols.

### Example 7.—Determining Editing Window (Prophetic)

To examine the editing window regions, the cytosine showing the highest C-T conversion frequency in a specified sgRNA is normalized to 1, and other cytosines at positions spanning from 30 nt upstream to 10 nt downstream of the PAM sequences (total 43 bp) of the same sgRNA are normalized subsequently. Then normalized C-T conversion frequencies are classified and compared according to their positions for all tested sgRNAs of a specified base editor. A comprehensive editing window (CEW) is defined to span positions with an average C-T conversion efficiency exceeding 0.6 after normalization.

To examine the substrate preference for each cytidine deaminase, C sites are initially classified according to their positions in sgRNA targeting regions and those positions containing at least one C site with >0.8 normalized C-T conversion frequency are included in subsequent analysis. Selected C sites are then compared depending on base types upstream or downstream of the edited cytosine (NC or CN). For cytidine deaminases showing efficient C-T conversion at both N-terminus and C-terminus of the endonuclease, the substrate preference is evaluated by integrating respective NT- and CT-CBEs together. For statistical analysis, one-way ANOVA is used and p<0.05 is considered as significant

### Example 8a.—Testing Off-Target Analysis with Whole Genome Sequencing and Transcriptomics (Prophetic)

Genomic DNA is fragmented and adapter-ligated using the Nextera DNA Flex Library Prep Kit (Illumina) using 96-well plate Nextera indexing primers (Illumina), according to the manufacturer's instructions. Library size and concentration is confirmed by Fragment Analyzer (Agilent) and DNA is sent to Novogene for WGS using an Illumina HiSeq system.

All targeted NGS data is analyzed by performing four general steps: (1) alignment; (2) duplicate marking; (3) variant calling and (4) background filtration of variants to remove artifacts and germline mutations. The mutation reference and alternate alleles are reported relative to the plus strand of the reference genome.

HEK293T cells are plated on 48-well poly-d-lysine-coated plates 16 to 20 h before lipofection at a density of 3.104 cells per well in DMEM+GlutaMAX medium (Thermo Fisher Scientific) without antibiotics. 750 ng nickase or base editor expression plasmid DNA is combined with 250 ng of sgRNA expression plasmid DNA in 15 μl Opti-MEM+GlutaMAX. This is combined with 10 μl of lipid mixture, comprising 1.5 μl Lipofectamine 2000 and 8.5 μl Opti-MEM+GlutaMAX per well. Cells are harvested 3 d after transfection and either DNA or RNA was harvested. For DNA analysis, cells are washed once in PBS, and then lysed in 100 μl QuickExtract Buffer (Lucigen) according to the manufacturer's instructions. For RNA harvest, the MagMAX mirVana Total RNA Isolation Kit (Thermo Fisher Scientific) is used with the KingFisher Flex

For whole Transcriptome sequencing, mRNA selection is performed using the NEBNext Poly(A) mRNA Magnetic Isolation Module (New England BioLabs). RNA library preparation is performed using NEBNext Ultra II RNA Library Prep Kit for Illumina (New England BioLabs). Based on the RNA input amount, a cycle number of 12 is used for the PCR enrichment of adapter-ligated DNA. NEBNext Sample Purification Beads (New England BioLabs) is used throughout for all of the size selection performed by this method. NEBNext Multiplex Oligos for Illumina (New England BioLabs) is used for the multiplex indexes in accordance with the PCR recipe outlined in the protocol. Prior to sequencing, samples are quality checked using the High Sensitivity D1000 ScreenTape on the 4200 TapeStation System (Agilent). The libraries are pooled and sequenced using a NovaSeq (Novogene). Targeted RNA sequencing is then performed. Complementary DNA is generated by PCR with reverse transcription (RT-PCR) from the isolated RNA using the SuperScript IV One-Step RT-PCR System with EZDnase (Thermo Fisher Scientific) according to the manufacturer's instructions.

The following program is used: 58° C. for 12 min; 98° C. for 2 min; followed by PCR cycles that varied by amplicon: for CTNNB1 and IP90; 32 cycles of (98° C. for 10 s; 60° C. for 10 sec; 72° C. for 30 sec). Following the combined RT-PCR, amplicons are barcoded and sequenced using an Illumina MiSeq sequencer as described above. The first 125 nucleotides in each amplicon, beginning at the first base after the end of the forward primer in each amplicon, are aligned to a reference sequence and used for analysis of maximum A-to-I frequencies in each amplicon. Off-target DNA sequencing is performed using primers, using a two-step PCR and barcoding method to prepare samples for sequencing using Illumina MiSeq sequencers as above.

### Example 8b.—Analysis of Off-Target Edits with Whole Genome Sequencing and Transcriptomics (Prophetic)

Transfected cells are harvested after 3 days and the genomic DNA isolated using the Agencourt DNAdvance Genomic DNA Isolation Kit (Beckman Coulter) according to the manufacturer's instructions. On-target and off-target genomic regions of interest are amplified by PCR with flanking HTS primer pairs. PCR amplification is carried out with Phusion high-fidelity DNA polymerase (ThermoFisher) according to the manufacturer's instructions using 5 ng of genomic DNA as a template. Cycle numbers are determined separately for each primer pair as to ensure the reaction was stopped in the linear range of amplification (30, 28, 28, 28, 32, and 32 cycles for EMX1, FANCF, HEK293 site 2, HEK293 site 3, HEK293 site 4, and RNF2 primers, respectively). PCR products are purified using RapidTips (Diffinity Genomics). Purified DNA is amplified by PCR with primers containing sequencing adaptors. The products are gel-purified and quantified using the Quant-iT™ PicoGreen dsDNA Assay Kit (ThermoFisher) and KAPA Library Quantification Kit-Illumina (KAPA Biosystems). Samples are sequenced on an Illumina MiSeq as previously described.

Sequencing reads are automatically demultiplexed using MiSeq Reporter (Illumina), and individual FASTQ files are analyzed with a custom Matlab script. Each read is pairwise aligned to the appropriate reference sequence using the Smith-Waterman algorithm. Base calls with a Q-score below 31 are replaced with N's and are thus excluded in calculating nucleotide frequencies. This treatment yields an expected MiSeq base-calling error rate of approximately 1 in 1,000. Aligned sequences in which the read and reference sequence contained no gaps are stored in an alignment table from which base frequencies could be tabulated for each locus. Indel frequencies were quantified with a custom Matlab script.

Sequencing reads are scanned for exact matches to two 10-bp sequences that flank both sides of a window in which indels might occur. If no exact matches were located, the read is excluded from analysis. If the length of this indel window exactly matched the reference sequence the read is classified as not containing an indel. If the indel window is two or more bases longer or shorter than the reference sequence, then the sequencing read is classified as an insertion or deletion, respectively.

### Example 9.—Mouse Editing Experiments (Prophetic)

It is envisaged that a base editor consisting of a novel DNA targeting nuclease domain fused to a novel deaminase domain could be validated as a therapeutic candidate by testing in appropriate mouse models of disease.

One example of an appropriate model comprises mice that have been engineered to express the human PCSK9 protein, for example, as described by Herbert et al (10.1161/ATVBAHA.110.204040). The PCSK9 protein regulates LDL receptor (LDLR) levels and influences serum cholesterol levels. Mice expressing the human PCSK9 protein exhibit elevated levels of cholesterol and more rapid development of atherosclerosis. PCSK9 is a validated drug target for the reduction of lipid levels in people at increased risk of cardiovascular disease due abnormally high plasma lipid levels (doi.org/10.1038/s41569-018-0107-8). Reducing the levels of PCSK9 via genome editing is expected to permanently lower lipid levels for the life-time of the individual thus providing a life-long reduction in cardiovascular disease risk. One genome editing approach would involve targeting the coding sequence of the PCSK9 gene with the goal of editing a sequence to create a premature stop codon and thus prevent the translation of the PCSK9 mRNA into a functional protein. Targeting a region close to the 5′ end of the coding sequence is preferred in order to block translation of the majority of the protein. To create a stop codon (TGA, TAA, TAG) with high efficiency and specificity will require targeting a region of the PCSK9 coding sequence wherein the editing window will be placed over an appropriate sequence such that the highest frequency editing event results in a stop codon. Therefore, the availability of multiple base editing systems with a wide range of PAMs or a base editing system with a degenerate PAM is desirable in order to access a larger number of potential target sites in the PCSK9 gene. In addition, the frequency of off-target editing should be low, e.g. in the range of 1% or less of the on-target editing events.

The efficiency of base editing required for a therapeutic effect is in the range of 50% or higher in order to achieve a significant reduction in plasma lipid levels. An example of the use of a base editor to create a stop codon in the PCSK9 gene is that of Carreras et al (doi.org/10.1186/s12915-018-0624-2) in which between 10% and 34% of the PCSK9 alleles were edited to create a stop codon. While this level of editing was sufficient to result in a measurable reduction in plasma lipid levels in the mice, a higher editing efficiency will be required for therapeutic use in humans.

To identify a base-editing (BE) system and a guide that are optimal for introducing the desired stop codons in the PCSK9 gene, a screen would be performed in a mouse liver cell line such as Hepa1-6 cells. In silico screening would first be used to identify guides that target the PCSK9 gene with the various BE systems available. To select among the large number of possible guides an in-silico analysis would be performed to determine which guides have an editing window that encompasses a sequence that when edited would create a stop codon. Preference would then be given to those guides that are closer to the 5′ end of the coding sequence. The resulting set of guides and BE proteins would be combined to form a ribonucleoprotein complex (RNP) and would be nucleofected into Hepa1-6 cells. After 72 h the efficiency of editing at the target site would be determined by NGS analysis. Based on these in vitro results the one or more BE/guide combinations that resulted in the highest frequency of stop codon formation would be selected for in vivo testing.

For application in a human therapeutic setting a safe and effective method of delivering the base editing components comprising the base editor and the guide RNA is required. In vivo delivery methods can be divided in to viral or non-viral methods. Among viral vectors the Adeno Associated Virus (AAV) is the virus of choice for clinical use due to its safety record, efficient delivery to multiple tissues and cell types and established manufacturing processes. The large size of base editors (BE) exceeds the packaging capacity of AAV which means that they cannot be packaged in a single Adeno Associated Virus. While approaches that package BE into two AAV using split intein technology have been demonstrated to be successful in mice (doi.org/10.1038/s41551-019-0501-5), the need for 2 viruses complicates development and manufacture. An additional disadvantage of AAV is that while the virus does not have a mechanism for promoting integration into the genome of host cells, and most of the AAV genomes remain episomal, a fraction of the AAV genomes do become integrated at random double strand breaks that occur naturally in cells (Curr Opin Mol Ther. 2009 August; 11(4): 442-447). This could lead to the persistence of gene sequences expressing the BE for the life-time of the organism. Moreover, AAV genomes persist as episomes inside the nucleus of transduced cells and can be maintained for years which would result in the long-term expression of BE in these cells and thus an increased risk of off-target effects because the risk of an off-target event occurring is a function of the time over which the editing enzyme is active. Adenovirus (Ad) such as Ad5 can efficiently deliver DNA payloads to the liver of mammals and can package up to 45 kb of DNA. However adenoviruses are known to induce a strong immune response in mammals (dx.doi.org/10.1136/gut.48.5.733), including in patients which can result in serious adverse events including death (doi.org/10.1016/j.ymthe.2020.02.010).

Non-viral delivery vectors (reviewed in doi:10.1038/mt.2012.79) which include lipid nanoparticles and polymeric nanoparticles have several advantages compared to viral delivery vectors including lower immunogenicity and transient expression of the nucleic acid cargo. The transient expression elicited by non-viral delivery vectors is particularly suited to genome editing applications because it is expected to minimize off target events. In addition non-viral delivery unlike viral vectors has the potential for repeat administration to achieve the desired therapeutic effect. There is also no theoretical limit to the size of the nucleic acid molecules that can be packaged in non-viral vectors, although in practice the packaging becomes less efficient as the size of the nucleic acid increases and the particles size may increase.

A BE could be delivered in vivo using a non-viral vector such as a lipid nanoparticle (LNP) by encapsulating a synthetic mRNA encoding the BE together with the guide RNA into the LNP. This can be performed using methodologies well known in the art, for example as described by Finn et al (DOI: 10.1016/j.celrep.2018.02.014) or Yin et al (doi:10.1038/nbt.3471). Typically, LNP deliver their cargo primarily to the hepatocytes of the liver which is a desired target organ/cell type when attempting to interfere with the expression of the PCSK9 gene. In order to demonstrate proof of concept for this approach we envisage that a BE comprised of a novel genome editing protein fused to a deaminase domain would be encoded in a synthetic mRNA and packaged in a LNP together with an appropriate guide RNA that targets the selected site in the PCSK9 gene of the mouse. In the case of mice that were engineered to express the human PCSK9 gene the guide would be designed to target only the human PCSK9 gene or both the human and mouse PCSK9 genes. Following injection of these LNP the editing efficiency at the on-target site in the genome of the liver cells would be analyzed by amplicon sequencing or other methods such as tracking of indels by decomposition (doi: 10.1093/nar/gku936). The physiologic impact would be determined by measuring lipid levels in the blood of the mice, including total cholesterol and triglyceride levels using standard methods.

Another example of a disease that could be modeled in mice to evaluate a novel BE is Primary Hyperoxaluria type I. Primary Hyperoxaluria type I (PH1) is a rare autosomal recessive disease caused by defects in the AGXT gene that encodes the enzyme alanine-glyoxylate aminotransferase. This results in a defect in glyoxylate metabolism and the accumulation of the toxic metabolite oxalate. One approach to treating this disease is to reduce the expression of the enzyme glycolate oxidase (GO) that produces glyoxylate from glycolate and thereby reducing the amount of substrate (glyoxylate) available for the formation of oxalate. PH1 can be modeled in mice in which both copies of the AGXT gene have been knocked out (agxt−/− mice) resulting in a significant 3-fold increase in oxalate levels in the urine compared to wild type controls. The agxt−/− mice can therefore be used to assess the efficacy of a novel base editor designed to create a stop codon in the coding sequence of the endogenous mouse GO gene. To identify a BE system and a guide that is optimal for introducing the desired stop codons in the GO gene, a screen would be performed in a mouse liver cell line such as Hepa1-6 cells. In silico screening would first be used to identify guides that target the GO gene with the various BE systems available. To select among the large number of possible guides a in silico analysis would be performed to determine which guides have an editing window that encompasses a sequence that when edited would create a stop codon. Preference would then be given to those guides that are closer to the 5′ end of the coding sequence. The resulting set of guides and BE proteins would be combined to form a ribonucleoprotein complex (RNP) and would be nucleofected in to Hepa1-6 cells. After 72 h, the efficiency of editing at the target site would be determined by NGS analysis. Based on these in vitro results the one or more BE/guide combinations that resulted in the highest frequency of stop codon formation would be selected for in vivo testing in mice.

The BE and guide could be delivered to the mice using an AAV virus with a split intein system to express the BE and a 3rd AAV to deliver the guide. Alternatively, an Adenovirus type 5 could be used to deliver the BE and guide in a single virus because of the >40 Kb packaging capacity of Adenovirus. However, the preferred approach would be to deliver the BE as a mRNA together with the guide RNA packaged in an appropriate LNP. After intravenous injection of the LNP into the agxt−/− mice the oxalate levels in the urine would be monitored over time to determine if oxalate levels were reduced which would indicate that the BE was active and had the expected desired therapeutic effect. To determine if the BE had introduced the desired stop codons, the appropriate region of the GO gene would be PCR amplified from the genomic DNA extracted from livers of treated and control mice. The resultant PCR product would be sequenced using Next Generation Sequencing to determine the frequency of the desired sequence changes.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-138. (canceled)

139. An engineered nucleic acid editing system, comprising:
a. an endonuclease comprising a RuvC domain and an HNH domain, wherein said endonuclease is derived from an uncultivated microorganism, wherein said endonuclease is a class 2, type II Cas endonuclease, wherein said endonuclease comprises an amino acid sequence with at least 80% sequence identity to SEQ ID NO: 23, and wherein said endonuclease is configured to be deficient in nuclease activity;
b. a base editor coupled to said endonuclease; and
c. an engineered guide ribonucleic acid structure configured to form a complex with said endonuclease comprising:
i. a guide ribonucleic acid sequence configured to hybridize to a target deoxyribonucleic acid sequence; and
ii. a non-guide ribonucleic acid sequence configured to bind to said endonuclease.

140. The engineered nucleic acid editing system of claim 139, wherein said endonuclease comprises an amino acid sequence with at least 90% sequence identity to SEQ ID NO: 23.

141. The engineered nucleic acid editing system of claim 139, wherein said endonuclease comprises an amino acid sequence with at least 95% sequence identity to SEQ ID NO: 23.

142. The engineered nucleic acid editing system of claim 139, wherein said endonuclease comprises an amino acid sequence of SEQ ID NO: 23.

143. The engineered nucleic acid editing system of claim 139, wherein said endonuclease comprises an aspartate to alanine mutation at residue 9 relative to SEQ ID NO: 23 when optimally aligned.

144. The engineered nucleic acid editing system of claim 139, wherein said endonuclease comprises a nickase mutation.

145. The engineered nucleic acid editing system of claim 139, wherein said RuvC domain lacks nuclease activity.

146. The engineered nucleic acid editing system of claim 139, wherein said endonuclease is configured to cleave one strand of a double-stranded target deoxyribonucleic acid.

147. The engineered nucleic acid editing system of claim 139, wherein said endonuclease comprises an amino acid sequence comprising less than 80% sequence identity to a Cas9 endonuclease.

148. The engineered nucleic acid editing system of claim 139, wherein said non-guide ribonucleic acid sequence comprises a tracr sequence.

149. The engineered nucleic acid editing system of claim 148, wherein said tracr sequence comprises at least 80% sequence identity to about 60 to 90 consecutive nucleotides of SEQ ID NO: 29.

150. The engineered nucleic acid editing system of claim 148, wherein said tracr sequence comprises at least 80% sequence identity to SEQ ID NO: 29.

151. The engineered nucleic acid editing system of claim 139, wherein said endonuclease is configured to bind to a protospacer adjacent motif (PAM) sequence of SEQ ID NO: 147.

152. The engineered nucleic acid editing system of claim 139, wherein said base editor comprises a sequence with at least 70% sequence identity to any one of SEQ ID NOs: 1-17.

153. The engineered nucleic acid editing system of claim 139, wherein said base editor is an adenosine deaminase.

154. The engineered nucleic acid editing system of claim 153, wherein said adenosine deaminase comprises a sequence with at least 80% sequence identity to any one of SEQ ID NOs: 8 or 164.

155. The engineered nucleic acid editing system of claim 139, wherein said base editor is a cytosine deaminase.

156. The engineered nucleic acid editing system of claim 155, wherein said cytosine deaminase comprises a sequence with at least 80% sequence identity to any one of SEQ ID NOs: 1-7 or 9-17.

157. The engineered nucleic acid editing system of claim 139, wherein said guide ribonucleic acid sequence is complementary to a prokaryotic, bacterial, archaeal, eukaryotic, fungal, plant, mammalian, or human genomic sequence.

158. The engineered nucleic acid editing system of claim 139, wherein said guide ribonucleic acid sequence is 15-24 nucleotides in length.

159. The engineered nucleic acid editing system of claim 139, wherein said endonuclease or said base editor comprises one or more nuclear localization sequences (NLSs).

160. The engineered nucleic acid editing system of claim 139, wherein said endonuclease is covalently coupled directly to said base editor or covalently coupled to said base editor through a linker.

161. The engineered nucleic acid editing system of claim 139, wherein said sequence identity is determined by a BLASTP, CLUSTALW, MUSCLE, MAFFT, or Smith-Waterman homology search algorithm.

162. The engineered nucleic acid editing system of claim 139, wherein said sequence identity is determined by BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

163. The engineered nucleic acid editing system of claim 139, further comprising a uracil deoxyribonucleic acid glycosylase inhibitor.

164. The engineered nucleic acid editing system of claim 163, wherein said uracil deoxyribonucleic acid glycosylase inhibitor comprises a sequence with at least 70% sequence identity to SEQ ID NO: 18.


SEQUENCE LISTING (163 sequences)

SEQ ID NO: 1 - protein, 217 aa, unidentified
  REGION 1..217: note = Description of Unknown: uncultivated organism
  REGION 1..217: note = Category: MG66 putative cytidine deaminase
  REGION 1..217: note = Description: MG66-1 deaminase
MKRIVTGTNDSCPLTGALEYAKVDKYTRQTLASMGITRMEDFKAFEEHQFLSQPGIGPKRFSQVTALMEAFNLRWKKPDPMNRPGWDEYFTRIASVVATRSHDEQTQVGCVVVDRDRRIRATGYNGFPPGFPDDALPKTRPEKYPFMVHAELNAIASSNQSLKRCTLYCTHSPCGNCVKAIITTGIERVVFRTAYTDWEESRKLLDLGRLEYYMISD

SEQ ID NO: 2 - protein, 244 aa, unidentified
  REGION 1..244: note = Description of Unknown: uncultivated organism
  REGION 1..244: note = Category: MG66 putative cytidine deaminase
  REGION 1..244: note = Description: MG66-16 deaminase
MEMTKEYTYSSGYNVIDNEIDFKCKFNEEWVSYFMNIARVVATKSKDPNTKVGCVVVDTKTKRIMATGYNGFPPGVNEDKSRWERPTKYDFVTHAEANCIAAAARFGIGLAGATMFVTLHPCVDCAKLIASAGISNIAFIESELERKQDRDWITHLENAKAIFRESRINLIAIREVKPIADVLEPQPLSFKGSVATVDDLNTIDPIIGDVFYVNSENTEYVYWLGKWGLLHETFKDPNRFPYKQ

SEQ ID NO: 3 - protein, 131 aa, unidentified
  REGION 1..131: note = Description of Unknown: uncultivated organism
  REGION 1..131: note = Category: MG66 putative cytidine deaminase
  REGION 1..131: note = Description: MG66-17 deaminase
MKNSESHPIQSKLERPGWDRYFMDIAKAVSARSIDPSVHVGAVIVNSGRRILSTGYNGFPPGFPDDELPLTRPEKYPYTVHAEVNAIASSQSDLRGGTLYCTLSPCVECTKVIITSGIQCVVFEEKYATSE

SEQ ID NO: 4 - protein, 146 aa, unidentified
  REGION 1..146: note = Description of Unknown: uncultivated organism
  REGION 1..146: note = Category: MG67 putative cytidine deaminase
  REGION 1..146: note = Description: MG67-1 deaminase
MKVQVVNEIGATEIQIKSYLTLCKNAQKAMQNAYSEYSGFKVGAALLLKNGEVVQSCNVENASYGLTMCAERAAIAKAVSEGFKPGDLDAIAISSNGKDFSPCGACRQVIHEFGVDVVVIFEFEGNVVITTASKMLPYGYSEEKQS

SEQ ID NO: 5 - protein, 137 aa, unidentified
  REGION 1..137: note = Description of Unknown: uncultivated organism
  REGION 1..137: note = Category: MG67 putative cytidine deaminase
  REGION 1..137: note = Description: MG67-3 deaminase
MKEAYNGLLQHAHRAKENAHAPYSGFKVGAAILAKDGRIIEGCNVENVAYGSTMCAERVAIYKAVSMGYKPGDFDAIAIASTGKNFSPCGACRQVINEFGDDINIIFEWEEKIVTESAKSMLPYNFESQTASPHHKS

SEQ ID NO: 6 - protein, 137 aa, unidentified
  REGION 1..137: note = Description of Unknown: uncultivated organism
  REGION 1..137: note = Category: MG67 putative cytidine deaminase
  REGION 1..137: note = Description: MG67-5 deaminase
MKEAYKELLQHAHRAKENAHAPYSGFKVGAAILAKDGQIIEGCNVENVAYGSTMCAERVAIYKAVSMGYKPGDFDAIAIASTGKNFSPCGSCRQVINEFGDDIDIIFEWEEKIVTESAKSMLPYNFESQTASPHHKS

SEQ ID NO: 7 - protein, 137 aa, unidentified
  REGION 1..137: note = Description of Unknown: uncultivated organism
  REGION 1..137: note = Category: MG67 putative cytidine deaminase
  REGION 1..137: note = Description: MG67-6 deaminase
MKEEYKALLQHAHQAKENAHAPYSGFKVGAAILAKDGRIIEGCNVENVAYGSTMCAERVAIYKAVSMGYKPGDFDAIAIASTGKNFSPCGACRQVINEFGDDINIIFEWEEKIVTESAKSMLPYNFESQTASPHHKS

SEQ ID NO: 8 - protein, 167 aa, Escherichia coli
  REGION 1..167: note = Category: reference deaminase
  REGION 1..167: note = Description: P68398 TADA tRNA specific adenosine deaminase
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 9 - protein, 229 aa, Rattus norvegicus [sequence omitted]

SEQ ID NO: 10 - protein, 251 aa, Heterocephalus glaber
  REGION 1..251: note = Category: reference deaminase
  REGION 1..251: note = Description: Aicda XM 004869540 cytidine deaminase
MAVGSKPKAALVGPHWERERIWCFLCSTGLGTQQTGQTSRWLRPAATQDPVSPPRSLLMKQRKFLYHFKNVRWAKGRHETYLCYVVKRRDSATSFSLDFGYLRNKSGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGL

SEQ ID NO: 11 - protein, 224 aa, Petromyzon marinus
  REGION 1..224: note = Category: reference deaminase
  REGION 1..224: note = Description: PmCDA1 L1 AVN88313.1 cytidine deaminase
MAGYECVRVSEKLDFDTFEFQFENLHYATERHRTYVIFDVKPQSAGGRSRRLWGYIINNPNVCHAELILMSMIDRHLESNPGVYAMTWYMSWSPCANCSSKLNPWLKNLLEEQGHTLTMHFSRIYDRDREGDHRGLRGLKHVSNSFRMGVVGRAEVKECLAEYVEASRRTLTWLDTTESMAAKMRRKLFCILVRCAGMRESGIPLHLFTLQTPLLSGRVVWWRV

SEQ ID NO: 12 - protein, 208 aa, Petromyzon marinus
  REGION 1..208: note = Category: reference deaminase
  REGION 1..208: note = Description: PmCDA1 ABO15149.1 cytosine deaminase
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 13 - protein, 199 aa, Homo sapiens
  REGION 1..199: note = Category: reference deaminase
  REGION 1..199: note = Description: NP 663745.1 DNA dC- dU-editing deaminase APOBEC-3A isoform a
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 14 - protein, 198 aa, Homo sapiens [sequence omitted]

SEQ ID NO: 15 - protein, 197 aa, Lampetra planeri
  REGION 1..197: note = Category: reference deaminase
  REGION 1..197: note = Description: LpCDA1L1 3 AVN88320.1 cytidine deaminase
MAGDENVRVSEKLHFNTFEFEFENLHYAEGRGQTYVIFDVKPQSEGGRGERLWGYVRNKPLGDHAEVILMSKINDHLETHQDNYTMTWYMSWSPCGKCSSELVPWLQNLLKKQHKLTMHFSRIYDKDRAVDHRGLCDLQHVVSNGFQMGVMGQTEVDTCLAEYVEASGCPPLKWLHMTDSNATQMQDKLSSILMNRF

SEQ ID NO: 16 - protein, 198 aa, Lampetra planeri
  REGION 1..198: note = Category: reference deaminase
  REGION 1..198: note = Description: LpCDA1L1 1 AVN88319.1 cytidine deaminase
MAGDENVRVSKKLDFNTFEFEFENLHYAEGRGRTYVIFDVKPQSEGGRGERLWGYVRNNPLDDHAEVILMSKINDHLETHQGNYTMTWYMSWSPCGNCSSELVPWLQNLLEEQQHTLTMYFSRIYDKDRAVDHRGLCDLQHVVSNGFQMGVMGQTEVDTCLAEYVEASGCPPLKWLHMTDSNATQTQDKLSSILMNRF

SEQ ID NO: 17 - DNA, 642 bp, Lampetra planeri
  misc_feature 1..642: note = Category: reference deaminase
  misc_feature 1..642: note = Description: ljCDA1 cytidine deaminase
  source 1..642: mol_type = other DNA
gccaacgccgagtacgtgcgcgtgggcgagaagctggacagctgcaccttccgcacccagttcctgaactaccgccgcagccgcagccgccgctgctgcgtgatcttcgagctgaagcgccagaacagccgcgtgcgcttctggggctacgccatgaacaagccctggagcaacgccgacgtgggcatccacgccgagttcttctgcatcaagaaggtgaagaagtacctgcgcaagaaccccggcatctacaccatcaactggtacagcagctggagcccctgcgccaactgcgccgagaagatcctgaactggtacaacaagaagctgatgggcaagggccacaccctgaagatttgggcctgcaagctgtacttcgagaacatcaagcgcaaccagatcggcctgtggaacctgcgcaacaacggcgtgggcctggccatcatgctgggcgagcactaccagtggtgctggaacaactacatccagaccctgggccgcaacctgaacgagaacaagtggctgaaaaagaccagcaaccgcgcccgcacccgccgcagcgagctgagcatcatgatccacgtgaagcgcctgcacaccgcccgcctgctgctgttcaagcgcctgtgcggctggttcagc

SEQ ID NO: 18 - protein, 84 aa, unidentified
  REGION 1..84: note = Description of Unknown: Bacillus phage PBS2 sequence
  REGION 1..84: note = Category: reference UGI
  REGION 1..84: note = Description: P14739 UNGI BPPB2 (UGI)
MTNLSDIIEKETGKQLVIQESILMLPEEVEEVIGNKPESDILVHTAYDESTDENVMLLTSDAPEYKPWALVIQDSNGENKIKML

SEQ ID NO: 19 - protein, 1245 aa, synthetic construct
  REGION 1..1245: note = Description of Artificial Sequence: Synthetic polypeptide
  REGION 1..1245: note = Category: adenine base editor
  REGION 1..1245: note = Description: linker-His tag-adenine deaminse-linker-nickase -linker-SV40 NLS
MGSSHHHHHHSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNAKTGAAGSLMDVLHYPGMNHRVEITEGILADECAALLCYFFRMPRRVFNAQKKAQSSTDSGGSSGGSSGSETPGTSESATPESSGGSSGGSMKKKVFAFALGKASIGYCVRDGFNINEANSIIIDKDHAETMSLRDRRRIKKTLDAHHAREEYFNKVWSGVGLNILDKNNNNFKKEFPSKNEDVIYTSCLLRIALLQNQKLEEWQIYKALYNAFQRRGYDVNIAWKSVQTDDDKENIERMKKYTCENDIELIISDEYKYPCYYDALRLGLWNEDEPTKLNRSIPQHNSNKVRTTEYVAPRELVEKELKQLWLNAQLQLPQLNSISAEEFLYGEYKIPYGSCVQKEFKQYRGTKKDWQGVLCQKIPRFDNRIIAKCKLLPKRNVCKANTIENVSFVLLMKLKNLRFTVISGEKLRLSPVQIKQIYENWLEKVEDTYNKDLEKAEKLGKENPEKRLNITITRQEIEKIIGEKIIDKIEPMKADISGRSSFCRRACQIMNKIILDGELYPQNVDISEFVDRDNAKNGITEEEIQSMLSKIGDWNNLYIPDNREENAQNASNSRIKTDIMLGNITNPIVRNRLQIFRDLLLNLAEKYGTPDEVIFEFVRDGADNSLYGSKKAQDTERFMKQQEKENEQIINELKDAGLYVNGQNHANATYFLMHKLLKSQGGKCIYSGQNISPSNYDECEIDHIYPRTLGGNDALYNKVVCYRRENQDKKGRTPYEWLSPDSDKWANYVNRLNKIKKQLGKKKFELLTSKPEDCEKLIESYNGLAETSHIARVAQQMTAFIFGWGLQIQGESRRIFVNNGSSTCAIRKRYGLNKLLGDNLKKNRSNDKHHALDAICISFSRDFKYDKEIKKDIIKGFNPEIVKNAIDKIMPYPYANDKPFKGNTKPLETIYGLRTYGDKSYITQRVELNSIDKKATKIKSIIDETIKNDLLNKLKENPTEQEWKLMLQNYIHPKKQTKVKKVMISVSEGEITKDSNNRERMGEFVDFGTKGTQHQFKHSKRHKGQILYFNEKGVVEVMPVYSNIKTTDVKDKLQNMGCKLYNKGQMFYSGCLVDIPKPFKAGSKEYPAGRYQIKTIRSDKVAELEDACGNKISTNVKYLVPAEFKKVESKGSGGSPKKKRKV

SEQ ID NO: 20 - protein, 1380 aa, synthetic construct
  REGION 1..1380: note = Description of Artificial Sequence: Synthetic polypeptide
  REGION 1..1380: note = Category: cytosine base editor
  REGION 1..1380: note = Description: linker-His tag-cytidine deaminase-linker-nickase -linker-uracil glycosylase inhibitor-linker-SV40 NLS
MGSSHHHHHHMSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESAMKKKVFAFALGKASIGYCVRDGFNINEANSIIIDKDHAETMSLRDRRRIKKTLDAHHAREEYFNKVWSGVGLNILDKNNNNFKKEFPSKNEDVIYTSCLLRIALLQNQKLEEWQIYKALYNAFQRRGYDVNIAWKSVQTDDDKENIERMKKYTCENDIELIISDEYKYPCYYDALRLGLWNEDEPTKLNRSIPQHNSNKVRTTEYVAPRELVEKELKQLWLNAQLQLPQLNSISAEEFLYGEYKIPYGSCVQKEFKQYRGTKKDWQGVLCQKIPRFDNRIIAKCKLLPKRNVCKANTIENVSFVLLMKLKNLRFTVISGEKLRLSPVQIKQIYENWLEKVEDTYNKDLEKAEKLGKENPEKRLNITITRQEIEKIIGEKIIDKIEPMKADISGRSSFCRRACQIMNKIILDGELYPQNVDISEFVDRDNAKNGITEEEIQSMLSKIGDWNNLYIPDNREENAQNASNSRIKTDIMLGNITNPIVRNRLQIFRDLLLNLAEKYGTPDEVIFEFVRDGADNSLYGSKKAQDTERFMKQQEKENEQIINELKDAGLYVNGQNHANATYFLMHKLLKSQGGKCIYSGQNISPSNYDECEIDHIYPRTLGGNDALYNKVVCYRRENQDKKGRTPYEWLSPDSDKWANYVNRLNKIKKQLGKKKFELLTSKPEDCEKLIESYNGLAETSHIARVAQQMTAFIFGWGLQIQGESRRIFVNNGSSTCAIRKRYGLNKLLGDNLKKNRSNDKHHALDAICISFSRDFKYDKEIKKDIIKGFNPEIVKNAIDKIMPYPYANDKPFKGNTKPLETIYGLRTYGDKSYITQRVELNSIDKKATKIKSIIDETIKNDLLNKLKENPTEQEWKLMLQNYIHPKKQTKVKKVMISVSEGEITKDSNNRERMGEFVDFGTKGTQHQFKHSKRHKGQILYFNEKGVVEVMPVYSNIKTTDVKDKLQNMGCKLYNKGQMFYSGCLVDIPKPFKAGSKEYPAGRYQIKTIRSDKVAELEDACGNKISTNVKYLVPAEFKKVESKGSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 21 - protein, 1043 aa, synthetic construct
  REGION 1..1043: note = Description of Artificial Sequence: Synthetic polypeptide
  REGION 1..1043: note = Category: nickase
  REGION 1..1043: note = Description: nMG4-2 (D28A) nickase
MLREPGNSVKSKIMGQQAKRRSYVLGLAIGTHSVGWALLKFRDGRPCGVERAGVRIFEPGVEEVAFERGRAEPPGQKRRQARALRRQTERRARRKAKLLHILQRAGLLPKGEADEILPALDRDILARHSAAWPGARDALPYWLRSGALDHRLEPHEFGRALYHLGQRRGFLSNRRAPMRKNEEDGKVKAGISTLKEQMEKAGARTLGEFFAGLDPHQERIRQRYTSREMYEQEFEAVWSAQAAHHPAILTDDLKARVHHAVFHQRPLHNQSYLAGSCTLEPDRKRTPWACLIAQRFRMLQKLNDTRVLPASGPERPLSDEERQTVLTELDRKKELKFDRVRKLLGLSADSSFNWESGGEDRLVGNTTNARLAKVFGKRWWSLSPDDRDQVVEDVRSYEKAEALARRGREHWGLDEKAAGELSKLSLEDGYCRLSRQAIERLLPGMEKGTAYMELVRKLYPDRWAAGKPVDLLPALAETDLDMRNPVVRRCLTELRKVVNAVVRHYGKPSAIRIELARDLRKSAKQREQTWRRNRRNQQDREAAAEKLLQEARIANPSRADVEKVLLAEECGWHCPYTGHGFGMADLFGPHPHFDVEHIVPFSRSLDNSFLNKTICEARENRDRKRNHTPYEAYGADAERWDQIIARVQSFRGTASREKLRRFQQHEVEDLDGVAQRELNDTRYASLLAVQYVGMLYGGAVDAGHVRRVQAAKGGTTGYLRDMYGLGFVLGEGRKERSDHRHHAVDAVAIALTDPAALKSISQAASDERRGGRVSFGAVALPWVDFIGDVQAAIEAINVSHRPSRKVNGALHEETFYGPRGMDGDGRPTGYVQRKPVERLSAKEIPNIPDPAVREAVQAKLDEVGGTPAQAFKDPANHPVRKRGIPVHKVRLRLNINPVQVGSGATERHVLTGSNHHMEIIEVRDAKGGKKWTGRLVHRLEAKRRALGRETIVDRAVQAGRQFQFSLSPGDMIELTGEDGERKLHVVRSISEGRIEYVDARDARKKADIRASGDWRKPAVGSLLRLHCRKVVVTPFGEIRYAND

SEQ ID NO: 22 - protein, 1168 aa, synthetic construct
  REGION 1..1168: note = Description of Artificial Sequence: Synthetic polypeptide
  REGION 1..1168: note = Category: nickase
  REGION 1..1168: note = Description: nMG7-1 (D10A) nickase
MSNKTILGLALGVSSIGWAIIERNDENGRIVKSGVRVIPSSKSELSVFKDFDKGKPASFSKERTEKRGIRRSYFRKKLRRAKLIEHLKENNMFDPELLGPKYSIDVWEWREKATKEKITLAQLGRVLLHINQKRGYKSNRKAIVDEESDSNWLNAINDNSKLLREKGITVGEYFYQEGKLHERKPKVKFALHFRMKVRIFNRKDYLDEIEQIWKKQSEFYPELTDELKESIIDHTIFYQRPLKSAKHLLSECRYEKMHKVIARSNPLFQLFRVLEKVNNLRAEDAFGNNREITDEEKLKIIEACTSAQSWKLLDKKKNLSKSKIKSILGLGKDYEINLDSIEGSKTLHSIWEVLMKSWGEAGDWIDFDWSIQGNDFSKQKSYQLWHALYSIDEPQYLRKKLCEGFGFDLDTARLLMNIRLESDYGALSARAIKRIIPELLKFPKDATKAIENAGYKFTDSETKEERESRELKDRIEHLKKGALRNPVVEKVLNQLVTLVNAIYAHPELPNPDEIRVELARELKSGAKERRRAELGMARAAKDNDRIRELLQTEFGIPYPGRRDILRYRFWEEQDMRCVYSGDVIPRNKLFVGEEYELDHIIPRARLFNDSNSNLVLVKSSENKDKSDMTAADYMKSKGEKAFEEYLVRVKNLYDKGAKKKAGERGSGINKGKRNFLIMKKEEIPQDFIERQLRESQYIVKEAVKLLKEVCRDVTTTTGKITDLLKHQWGANDVFRNIQVPKYRKWGMTETIVDRKTGEVIERIIDWSKRKDHRHHALDAIIVACTRQSYIQQLNRLNVLYENDYESLKSYRKFELPWPSFHNDLISSLESLLVSFRNKRRVATMNKNRIKVGGKKKYIVQKTLTPRDAFHLETTYGRRLVNNYKLVKLNKKFSMELAELVIDPDLKEKILNRLMEFGNDPQKAFANLKKNPFKWKNENLEEVLIYDEVFTTRKKLDEKFNNPSEIIDPEVREIVTQRLKEFDNNPKKAFADIENKPVWYNKDKQIRIKTVNTRAKASDLYPVRTKENGNPKDFVFTRNNHHITVYQKEDGKYYDKVTSFWEAFELKKAKMPIYKENDDAAKAVLHLKINDMVLVDLNPEDLDQNDPEFFNTLSEHLYRVQKLASGDVTFRHHLETELSNKNTEVRVTNAESLYNRVVMYPLDVLGLPK

SEQ ID NO: 23 - protein, 1154 aa, synthetic construct
  REGION 1..1154: note = Description of Artificial Sequence: Synthetic polypeptide
  REGION 1..1154: note = Category: nickase
  REGION 1..1154: note = Description: nMG16-1 (D9A) nickase
MIKNILGLALGVGSIGWALIQTEDDQPKQIIGMGSRIVPLTKDDSDQFTKGQAISKNAERTARRTTRKGYDRYQLRRALLTQVLRQNGMLPECMDENMIDLWKLRSDAATEGKQLTLQQIGRVLYHINQKRGYKHAKSDDNGDSKQTKYVEAVNLRYKEIQEKNVTVGQHFYAELLNSKVESGNGPYYTFRIKDKVFPRAAYIAEFDQIMGVQKEYYPNVLTDELIETLRNRIIFYQRPLKSCKHLVGLCEFEMRPYKKDGKIVYGGPKCAPRTSPLAQLCAMWETVNNITLTNRNNERLEISNEQRRQLVQFLCTHETLKLTDLYKILGITKKDGWYGGKAIGKGIKGNVTLNQLRKALDGKYSQWLEMPIERIDVVDRNTAEAFWAVSPKVEETPLFQLWHAVYSLQNVEELTKTLQNRFSITDPQVIDALCKIDFVKPGYANKSHKFIRRLLPYLMEGMMYSEACACIQINHSNSMTKAEREARPLAERIELLQKNALRQPVIEKILNQMINLVNRLQQEYGPIDEARVELARELKQSREERKDAFDRNNKNEKRNKEISALISEQGIRPSRSRIQKYKMWEESEHRCMYCGKVVNLSEFLNGADVEIEHIIPRSILFDDSFSNKVCACRDCNREKDNMTAMDYMASKPEGEFEAYKQRVDEAFNAHRISKTKRDHLLWRRADIPQDFIDRQLRLSQYIATKAVEILQQGIRQVWTSGGGVTDFLRHQWGYDEILHTLNLPRYRQVEDLTEMVHYEHAGQEHDEERIKNWSKRIDHRHHAIDALTVALTRQSYIQRLNTLEASHEHMEKLVKEANTPYKEKKSLLEKWVALQPHFSVEEVTTQVDGILVSFRAGKRVTTPARRAVYHGGKRTIVQRGIQVPRGALTEDTIYGKLGDKFVVKYALDHPSMKPENIVDPTIRLLVENRITALGKKDAFKTPLYSAEGMEIKSVRCYTSLSEKGVVPIKYNEKGNAIGFAKKGNNHHVAIYKDQSGQYQEMVVSFWDAVERKLYGVPTVITNPKTVWDELLEKELPQDFLEKLPKDNWQYVLSMQENEMFVLGMEEDEFNDAIDTQDYNTLNKHLYRVQKLSHADYTFRFHTETKVDDKYDGVENGRNTSMSLKALVRIRSFNGLFTQFPHKVKIDIMGRITKA

SEQ ID NO: 24 - DNA, 30 bp, synthetic construct
  misc_feature 1..30: note = Description of Artificial Sequence: Synthetic oligonucleotide
  misc_feature 1..30: note = Category: target sequence
  misc_feature 1..30: note = Description: nMG4-2 (D28A) protospacer and PAM for in vitro nickase assay
  source 1..30: mol_type = other DNA
agccaccacgtcgcaagcctcgactgtccc

SEQ ID NO: 25 - DNA, 37 bp, synthetic construct
  misc_feature 1..37: note = Description of Artificial Sequence: Synthetic oligonucleotide
  misc_feature 1..37: note = Category: target sequence
  misc_feature 1..37: note = Description: nMG7-1 (D10A) protospacer and PAM for in vitro nickase assay
  source 1..37: mol_type = other DNA
ccgtgagccaccacgtcgcaagcctcgactagtcggg

SEQ ID NO: 26 - DNA, 37 bp, synthetic construct
  misc_feature 1..37: note = Description of Artificial Sequence: Synthetic oligonucleotide
  misc_feature 1..37: note = Category: target sequence
  misc_feature 1..37: note = Description: nMG16-1 (D9A) protospacer and PAM for in vitro nickase assay
  source 1..37: mol_type = other DNA
ccgtgagccaccacgtcgcaagcctcgactggacctg

SEQ ID NO: 27 - DNA, 127 bp, synthetic construct
  misc_feature 1..127: note = Description of Artificial Sequence: Synthetic polynucleotide
  misc_feature 1..127: note = Category: single guide RNA
  misc_feature 1..127: note = Description: nMG4-2 (D28A) single guide RNA for in vitro nickase assay
  source 1..127: mol_type = other DNA
ccaccacgtcgcaagcctcgacgctgtggtttgatgggttcgctcctcaaatcatagtaaggggcaattgcctcgcgggctctgccctacggggcacccctcgagcgcctcccagtggggcgctttt

SEQ ID NO: 28 - DNA, 109 bp, synthetic construct
  misc_feature 1..109: note = Description of Artificial Sequence: Synthetic polynucleotide
  misc_feature 1..109: note = Category: single guide RNA
  misc_feature 1..109: note = Description: nMG7-1 (D10A) single guide RNA for in vitro nickase assay
  source 1..109: mol_type = other DNA
ccaccacgtcgcaagcctcgacgttgtgaatggctttcagaaatgaagttattcacaataaggattattccgttgtgaaaacatttaaagcggccctcgggtcgctttt

SEQ ID NO: 29 - DNA, 100 bp, synthetic construct
  misc_feature 1..100: note = Description of Artificial Sequence: Synthetic polynucleotide
  misc_feature 1..100: note = Category: single guide RNA
  misc_feature 1..100: note = Description: nMG16-1 (D9A) single guide RNA for in vitro nickase assay
  source 1..100: mol_type = other DNA
ccaccacgtcgcaagcctcgacgttgtgtatggaaacatacacaataaggattattccgttgtgaaaacattcagggtgggacgcaagtctcgccctttt

SEQ ID NO: 30 - DNA, 22 bp, synthetic construct
  misc_feature 1..22: note = Description of Artificial Sequence: Synthetic oligonucleotide
  misc_feature 1..22: note = Category: spacer
  misc_feature 1..22: note = Description: MGA4-2 sgRNA spacer 1 (targeting E. coli lacZ)
  source 1..22: mol_type = other DNA
tgattaaatatgatgaaaacgg

SEQ ID NO: 31 - DNA, 22 bp, synthetic construct
  misc_feature 1..22: note = Description of Artificial Sequence: Synthetic oligonucleotide
  misc_feature 1..22: note = Category: spacer
  misc_feature 1..22: note = Description: MGA4-2 sgRNA spacer 2 (targeting E. coli lacZ)
  source 1..22: mol_type = other DNA
aacgacattggcgtaagtgaag

SEQ ID NO: 32 - DNA, 22 bp, synthetic construct
  misc_feature 1..22: note = Description of Artificial Sequence: Synthetic oligonucleotide
  misc_feature 1..22: note = Category: spacer
  misc_feature 1..22: note = Description: MGA4-2 sgRNA spacer 3 (targeting E. coli lacZ)
  source 1..22: mol_type = other DNA
acgcgcgaattgaattatggcc

SEQ ID NO: 33 - DNA, 22 bp, synthetic construct
  misc_feature 1..22: note = Description of Artificial Sequence: Synthetic oligonucleotide
  misc_feature 1..22: note = Category: spacer
  misc_feature 1..22: note = Description: MGA7-1 sgRNA spacer 1 (targeting E. coli lacZ)
  source 1..22: mol_type = other DNA
gaagatcaggatatgtggcgga

SEQ ID NO: 34 - DNA, 22 bp, synthetic construct
  misc_feature 1..22: note = Description of Artificial Sequence: Synthetic oligonucleotide
  misc_feature 1..22: note = Category: spacer
  misc_feature 1..22: note = Description: MGA7-1 sgRNA spacer 2 (targeting E. coli lacZ)
  source 1..22: mol_type = other DNA
aacgtcgaaaacccgaaactgt

SEQ ID NO: 35 - DNA, 22 bp, synthetic construct
  misc_feature 1..22: note = Description of Artificial Sequence: Synthetic oligonucleotide
  misc_feature 1..22: note = Category: spacer
  misc_feature 1..22: note = Description: MGA7-1 sgRNA spacer 3 (targeting E. coli lacZ)
  source 1..22: mol_type = other DNA
aaacccacggcatggtgccaat

SEQ ID NO: 36 - DNA, 22 bp, synthetic construct
  misc_feature 1..22: note = Description of Artificial Sequence: Synthetic oligonucleotide
  misc_feature 1..22: note = Category: spacer
  misc_feature 1..22: note = Description: MGA16-1 sgRNA spacer 1 (targeting E. coli lacZ)
  source 1..22: mol_type = other DNA
gccagctggcgtaatagcgaag

SEQ ID NO: 37 - DNA, 22 bp, synthetic construct
  misc_feature 1..22: note = Description of Artificial Sequence: Synthetic oligonucleotide
  misc_feature 1..22: note = Category: spacer
  misc_feature 1..22: note = Description: MGA16-1 sgRNA spacer 2 (targeting E. coli lacZ)
  source 1..22: mol_type = other DNA
agggtgaaacgcaggtcgccag

SEQ ID NO: 38 - DNA, 22 bp, synthetic construct
  misc_feature 1..22: note = Description of Artificial Sequence: Synthetic oligonucleotide
  misc_feature 1..22: note = Category: spacer
  misc_feature 1..22: note = Description: MGA16-1 sgRNA spacer 3 (targeting E. coli lacZ)
  source 1..22: mol_type = other DNA
ctacgtctgaacgtcgaaaacc

SEQ ID NO: 39 - DNA, 20 bp, synthetic construct
  misc_feature 1..20: note = Description of Artificial Sequence: Synthetic oligonucleotide
  misc_feature 1..20: note = Category: spacer
  misc_feature 1..20: note = Description: ABE8.17m sgRNA spacer 1 (targeting E. coli lacZ)
  source 1..20: mol_type = other DNA
tttctttcacagatgtggat

SEQ ID NO: 40 - DNA, 20 bp, synthetic construct
  misc_feature 1..20: note = Description of Artificial Sequence: Synthetic oligonucleotide
  misc_feature 1..20: note = Category: spacer
  misc_feature 1..20: note = Description: ABE8.17m sgRNA spacer 2 (targeting E. coli lacZ)
  source 1..20: mol_type = other DNA
caggatatgtggcggatgag

SEQ ID NO: 41 - DNA, 20 bp, synthetic construct
  misc_feature 1..20: note = Description of Artificial Sequence: Synthetic oligonucleotide
  misc_feature 1..20: note = Category: spacer
  misc_feature 1..20: note = Description: ABE8.17m sgRNA spacer 3 (targeting E. coli lacZ)
  source 1..20: mol_type = other DNA
tgcgaatacgcccacgcgat

SEQ ID NO: 42 - DNA, 22 bp, synthetic construct
  misc_feature 1..22: note = Description of Artificial Sequence: Synthetic oligonucleotide
  misc_feature 1..22: note = Category: spacer
  misc_feature 1..22: note = Description: MGC4-2 sgRNA spacer 1 (targeting E. coli lacZ)
  source 1..22: mol_type = other DNA
gccgtgcgctgttcgcattatc

SEQ ID NO: 43 - DNA, 22 bp, synthetic construct
  misc_feature 1..22: note = Description of Artificial Sequence: Synthetic oligonucleotide
  misc_feature 1..22: note = Category: spacer
  misc_feature 1..22: note = Description: MGC4-2 sgRNA spacer 2 (targeting E. coli lacZ)
  source 1..22: mol_type = other DNA
atactggcaggcgtttcgtcag

SEQ ID NO: 44 - DNA, 22 bp, synthetic construct
  misc_feature 1..22: note = Description of Artificial Sequence: Synthetic oligonucleotide
  misc_feature 1..22: note = Category: spacer
  misc_feature 1..22: note = Description: MGC4-2 sgRNA spacer 3 (targeting E. coli lacZ)
  source 1..22: mol_type = other DNA
cactccccgccgcgtcccacgc

SEQ ID NO: 45 - DNA, 22 bp, synthetic construct
  misc_feature 1..22: note = Description of Artificial Sequence: Synthetic oligonucleotide
  misc_feature 1..22: note = Category: spacer
  misc_feature 1..22: note = Description: MGC7-1 sgRNA spacer 1 (targeting E. coli lacZ)
  source 1..22: mol_type = other DNA
gccgatcgcgtcacactacgtc

SEQ ID NO: 46 - DNA, 22 bp, synthetic construct
  misc_feature 1..22: note = Description of Artificial Sequence: Synthetic oligonucleotide
  misc_feature 1..22: note = Category: spacer
  misc_feature 1..22: note = Description: MGC7-1 sgRNA spacer 2 (targeting E. coli lacZ)
  source 1..22: mol_type = other DNA
gatccgcgctggctaccggcga

SEQ ID NO: 47 - DNA, 22 bp, synthetic construct
  misc_feature 1..22: note = Description of Artificial Sequence: Synthetic oligonucleotide
  misc_feature 1..22: note = Category: spacer
  misc_feature 1..22: note = Description: MGC7-1 sgRNA spacer 3 (targeting E. coli lacZ)
  source 1..22: mol_type = other DNA
ccttcccgcccggtgcagtatg

SEQ ID NO: 48 - DNA, 22 bp, synthetic construct
  misc_feature 1..22: note = Description of Artificial Sequence: Synthetic oligonucleotide
  misc_feature 1..22: note = Category: spacer
  misc_feature 1..22: note = Description: MGC16-1 sgRNA spacer 1 (targeting E. coli lacZ)
  source 1..22: mol_type = other DNA
acgtgacctatcccattacggt

SEQ ID NO: 49 - DNA, 22 bp, synthetic construct
  misc_feature 1..22: note = Description of Artificial Sequence: Synthetic oligonucleotide
  misc_feature 1..22: note = Category: spacer
  misc_feature 1..22: note = Description: MGC16-1 sgRNA spacer 2 (targeting E. coli lacZ)
  source 1..22: mol_type = other DNA
atccgccgtttgttcccacgga

SEQ ID NO: 50 - DNA, 22 bp, synthetic construct
  misc_feature 1..22: note = Description of Artificial Sequence: Synthetic oligonucleotide
  misc_feature 1..22: note = Category: spacer
  misc_feature 1..22: note = Description: MGC16-1 sgRNA spacer 3 (targeting E. coli lacZ)
  source 1..22: mol_type = other DNA
cgacggcacgctgattgaagca

SEQ ID NO: 51 - DNA, 20 bp, synthetic construct
  misc_feature 1..20: note = Description of Artificial Sequence: Synthetic oligonucleotide
  misc_feature 1..20: note = Category: spacer
  misc_feature 1..20: note = Description: BE3 sgRNA spacer 1 (targeting E. coli lacZ)
  source 1..20: mol_type = other DNA
cctctggatgtcgctccaca

SEQ ID NO: 52 - DNA, 20 bp, synthetic construct
  misc_feature 1..20: note = Description of Artificial Sequence: Synthetic oligonucleotide
  misc_feature 1..20: note = Category: spacer
  misc_feature 1..20: note = Description: BE3 sgRNA spacer 2 (targeting E. coli lacZ)
  source 1..20: mol_type = other DNA
gggacgcgcgaattgaatta

SEQ ID NO: 53 - DNA, 20 bp, synthetic construct
  misc_feature 1..20: note = Description of Artificial Sequence: Synthetic oligonucleotide
  misc_feature 1..20: note = Category: spacer
  misc_feature 1..20: note = Description: BE3 sgRNA spacer 3 (targeting E. coli lacZ)
  source 1..20: mol_type = other DNA
cgttttacaacgtcgtgact

SEQ ID NO: 54 - DNA, 35 bp, synthetic construct
  misc_feature 1..35: note = Description of Artificial Sequence: Synthetic oligonucleotide
  misc_feature 1..35: note = Category: primer
  misc_feature 1..35: note = Description: Site-directed mutagenesis of MG4-2 (D28A)
  source 1..35: mol_type = other DNA
ctcctacgttttgggcttagcgatcggtacgcata

SEQ ID NO: 55 - DNA, 18 bp, synthetic construct
  misc_feature 1..18: note = Description of Artificial Sequence: Synthetic oligonucleotide
  misc_feature 1..18: note = Category: primer
  misc_feature 1..18: note = Description: Site-directed mutagenesis of MG4-2 (D28A)
  source 1..18: mol_type = other DNA
cggcgtttggcttgttga

SEQ ID NO: 56 - DNA, 40 bp, synthetic construct
  misc_feature 1..40: note = Description of Artificial Sequence: Synthetic oligonucleotide
  misc_feature 1..40: note = Category: primer
  misc_feature 1..40: note = Description: Site-directed mutagenesis of MG7-1 (D10A)
  source 1..40: mol_type = other DNA
gtaataaaacaattttaggcctggcgttaggtgttagttc

SEQ ID NO: 57 - DNA, 26 bp, synthetic construct
  misc_feature 1..26: note = Description of Artificial Sequence: Synthetic oligonucleotide
  misc_feature 1..26: note = Category: primer
  misc_feature 1..26: note = Description: Site-directed mutagenesis of MG7-1 (D10A)
  source 1..26: mol_type = other DNA
tcatggtatatctccttcttaaagtt

SEQ ID NO: 58 - DNA, 41 bp, synthetic construct
  misc_feature 1..41: note = Description of Artificial Sequence: Synthetic oligonucleotide
  misc_feature 1..41: note = Category: primer
  misc_feature 1..41: note = Description: Site-directed mutagenesis of MG16-1 (D9A)
  source 1..41: mol_type = other DNA
tgattaagaatatcttgggtctggcgctgggagtcggaagc

SEQ ID NO: 59 - DNA, 27 bp, synthetic construct
  misc_feature 1..27: note = Description of Artificial Sequence: Synthetic oligonucleotide
  misc_feature 1..27: note = Category: primer
  misc_feature 1..27: note = Description: Site-directed mutagenesis of MG16-1 (D9A)
  source 1..27: mol_type = other DNA
tggtatatctccttcttaaagttaaac

SEQ ID NO: 60 - DNA, 42 bp, synthetic construct
  misc_feature 1..42: note = Description of Artificial Sequence: Synthetic oligonucleotide
  misc_feature 1..42: note = Category: primer
  misc_feature 1..42: note = Description: Site-directed mutagenesis of SpCas9 (D10A)
  source 1..42: mol_type = other DNA
gataagaaatactcaataggactggcgattggcacaaatagc

SEQ ID NO: 61 - DNA, 25 bp, synthetic construct
  misc_feature 1..25: note = Description of Artificial Sequence: Synthetic oligonucleotide
  misc_feature 1..25: note = Category: primer
  misc_feature 1..25: note = Description: Site-directed mutagenesis of SpCas9 (D10A)
  source 1..25: mol_type = other DNA
catggtatatctccttcttaaagtt

SEQ ID NO: 62 - DNA, 21 bp, synthetic construct
  misc_feature 1..21: note = Description of Artificial Sequence: Synthetic oligonucleotide
  misc_feature 1..21: note = Category: primer
  misc_feature 1..21: note = Description: For lacZ sequencing
  source 1..21: mol_type = other DNA
ccaggctttacactttatgct

SEQ ID NO: 63 - DNA, 19 bp, synthetic construct
  misc_feature 1..19: note = Description of Artificial Sequence: Synthetic oligonucleotide
  misc_feature 1..19: note = Category: primer
  misc_feature 1..19: note = Description: For lacZ sequencing
  source 1..19: mol_type = other DNA
gtatgtggtggatgaagcc

SEQ ID NO: 64 - DNA, 18 bp, synthetic construct
  misc_feature 1..18: note = Description of Artificial Sequence: Synthetic oligonucleotide
  misc_feature 1..18: note = Category: primer
  misc_feature 1..18: note = Description: Amplify the fragment for nickase assay
  source 1..18: mol_type = other DNA
agcttccagggggaaacg

SEQ ID NO: 65 - DNA, 26 bp, synthetic construct
  misc_feature 1..26: note = Description of Artificial Sequence: Synthetic oligonucleotide
  misc_feature 1..26: note = Category: primer
  misc_feature 1..26: note = Description: Amplify the fragment for nickase assay
  source 1..26: mol_type = other DNA
attaacctataaaaataggcgtatca

SEQ ID NO: 66 - DNA, 60 bp, synthetic construct
  misc_feature 1..60: note = Description of Artificial Sequence: Synthetic oligonucleotide
  misc_feature 1..60: note = Category: primer
  misc_feature 1..60: note = Description: Amplify T7 promoter-His tag-adenine deaminase for MGA entry plasmid
  source 1..60: mol_type = other DNA
gatataccatgggcagcagtcatcatcatcaccatcactccgaggttgaatttagtcatg

SEQ ID NO: 67 - DNA, 60 bp, synthetic construct
  misc_feature 1..60: note = Description of Artificial Sequence: Synthetic oligonucleotide
  misc_feature 1..60: note = Category: primer
  misc_feature 1..60: note = Description: Amplify T7 promoter-His tag-adenine deaminase for MGA entry plasmid
  source 1..60: mol_type = other DNA
agactttcctcttcttcttgggagacccgccagagcctcgaggacccaccagagctgcct

SEQ ID NO: 68 - DNA, 55 bp, synthetic construct
  misc_feature 1..55: note = Description of Artificial Sequence: Synthetic oligonucleotide
  misc_feature 1..55: note = Category: primer
  misc_feature 1..55: note = Description: Amplify SV40 NLS-vector backbone for MGA entry plasmid
  source 1..55: mol_type = other DNA
tctggcgggtctcccaagaagaagaggaaagtctaagatccggctgctaacaaag

SEQ ID NO: 69 - DNA, 45 bp, synthetic construct
  misc_feature 1..45: note = Description of Artificial Sequence: Synthetic oligonucleotide
  misc_feature 1..45: note = Category: primer
  misc_feature 1..45: note = Description: Amplify SV40 NLS-vector backbone for MGA entry plasmid
  source 1..45: mol_type = other DNA
ctccttgcatgctctagaattaattaaaccattccttgcggcggc

SEQ ID NO: 70 - DNA, 48 bp, synthetic construct
  misc_feature 1..48: note = Description of Artificial Sequence: Synthetic oligonucleotide
  misc_feature 1..48: note = Category: primer
  misc_feature 1..48: note = Description: Amplify vector backbone for MGA entry plasmid
  source 1..48: mol_type = other DNA
aggaatggtttaattaattctagagcatgcaaggagatggcgcccaac

SEQ ID NO: 71 - DNA, 56 bp, synthetic construct
  misc_feature 1..56: note = Description of Artificial Sequence: Synthetic oligonucleotide
  misc_feature 1..56: note = Category: primer
  misc_feature 1..56: note = Description: Amplify vector backbone for MGA entry plasmid
  source 1..56: mol_type = other DNA
gtgatggtgatgatgatgactgctgcccatggtatatctccttcttaaagttaaac

SEQ ID NO: 72 - DNA, 60 bp, synthetic construct
  misc_feature 1..60: note = Description of Artificial Sequence: Synthetic oligonucleotide
  misc_feature 1..60: note = Category: primer
  misc_feature 1..60: note = Description: Amplify T7 promoter-His-tag-cytosine deaminase for MGC entry plasmid
  source 1..60: mol_type = other DNA
aggagatataccatgggcagcagtcatcatcatcaccatcacatgtcgtctgagaccggg

SEQ ID NO: 73 - DNA, 60 bp, synthetic construct
  misc_feature 1..60: note = Description of Artificial Sequence: Synthetic oligonucleotide
  misc_feature 1..60: note = Category: primer
  misc_feature 1..60: note = Description: Amplify T7 promoter-His-tag-cytosine deaminase for MGC entry plasmid
  source 1..60: mol_type = other DNA
taatatctgaaagattggtagaaccaccagacccgcggcagattcaggtgtcgcactttc

SEQ ID NO: 74 - DNA, 44 bp, synthetic construct
  misc_feature 1..44: note = Description of Artificial Sequence: Synthetic oligonucleotide
  misc_feature 1..44: note = Category: primer
  misc_feature 1..44: note = Description: Amplify UGI-SV40 NLS for MGC entry plasmid
  source 1..44: mol_type = other DNA
cgacacctgaatctgccgcgggtctggtggttctaccaatcttt

SEQ ID NO: 75 - DNA, 60 bp, synthetic construct
  misc_feature 1..60: note = Description of Artificial Sequence: Synthetic oligonucleotide
  misc_feature 1..60: note = Category: primer
  misc_feature 1..60: note = Description: Amplify UGI-SV40 NLS for MGC entry plasmid
  source 1..60: mol_type = other DNA
ttagactttcctcttcttcttgggagaaccaccagaaagcattttgatcttgttttcgcc

SEQ ID NO: 76 - DNA, 54 bp, synthetic construct
  misc_feature 1..54: note = Description of Artificial Sequence: Synthetic oligonucleotide
  misc_feature 1..54: note = Category: primer
  misc_feature 1..54: note = Description: Amplify SV40 NLS-vector backbone for MGC entry plasmid
  source 1..54: mol_type = other DNA
ctggtggttctcccaagaagaagaggaaagtctaagatccggctgctaacaaag

SEQ ID NO: 77 - DNA, 45 bp, synthetic construct
  misc_feature 1..45: note = Description of Artificial Sequence: Synthetic oligonucleotide
  misc_feature 1..45: note = Category: primer
  misc_feature 1..45: note = Description: Amplify SV40 NLS-vector backbone for MGC entry plasmid
  source 1..45: mol_type = other DNA
ctccttgcatgctctagaattaattaaccattccttgcggcggcg

SEQ ID NO: 78 - DNA, 48 bp, synthetic construct
  misc_feature 1..48: note = Description of Artificial Sequence: Synthetic oligonucleotide
  misc_feature 1..48: note = Category: primer
  misc_feature 1..48: note = Description: Amplify vector backbone for MGC entry plasmid
  source 1..48: mol_type = other DNA
aggaatggttaattaattctagagcatgcaaggagatggcgcccaaca

SEQ ID NO: 79 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify vector backbone for MGC entry plasmid
  source 1..50: mol_type = other DNA
gtgatgatgatgactgctgcccatggtatatctccttcttaaagttaaac

SEQ ID NO: 80 - DNA, 48 bp, synthetic construct
  misc_feature 1..48: note = Description of Artificial Sequence: Synthetic oligonucleotide
  misc_feature 1..48: note = Category: primer
  misc_feature 1..48: note = Description: Amplify nMG4-2 (D28A) for pMGA expression plasmid
  source 1..48: mol_type = other DNA
gaaagtagtggaggcagctctggtgggtccatgcttcgtgagcccggt

SEQ ID NO: 81 - DNA, 53 bp, synthetic construct
  misc_feature 1..53: note = Description of Artificial Sequence: Synthetic oligonucleotide
  misc_feature 1..53: note = Category: primer
  misc_feature 1..53: note = Description: Amplify nMG4-2 (D28A) for pMGA expression plasmid
  source 1..53: mol_type = other DNA
cctcttcttcttgggagacccgccagagccatcgttagcataacgaatttccc

SEQ ID NO: 82 - DNA, 57 bp, synthetic construct
  misc_feature 1..57: note = Description of Artificial Sequence: Synthetic oligonucleotide
  misc_feature 1..57: note = Category: primer
  misc_feature 1..57: note = Description: Amplify nMG7-1 (D10A) for pMGA expression plasmid
  source 1..57: mol_type = other DNA
gaaagtagtggaggcagctctggtgggtccatgagtaataaaacaattttaggcctg

SEQ ID NO: 83 - DNA, 51 bp, synthetic construct
  misc_feature 1..51: note = Description of Artificial Sequence: Synthetic oligonucleotide
  misc_feature 1..51: note = Category: primer
  misc_feature 1..51: note = Description: Amplify nMG7-1 (D10A) for pMGA expression plasmid
  source 1..51: mol_type = other DNA
cctcttcttcttgggagacccgccagagcctttgggcaagcctaaaacatc

SEQ ID NO: 84 - DNA, 54 bp, synthetic construct
  misc_feature 1..54: note = Description of Artificial Sequence: Synthetic oligonucleotide
  misc_feature 1..54: note = Category: primer
  misc_feature 1..54: note = Description: Amplify nMG16-1 (D9A) for pMGA expression plasmid
  source 1..54: mol_type = other DNA
gaaagtagtggaggcagctctggtgggtccatgattaagaatatcttgggtctg

SEQ ID NO: 85 - DNA, 48 bp, synthetic construct
  misc_feature 1..48: note = Description of Artificial Sequence: Synthetic oligonucleotide
  misc_feature 1..48: note = Category: primer
  misc_feature 1..48: note = Description: Amplify nMG16-1 (D9A) for pMGA expression plasmid
  source 1..48: mol_type = other DNA
cctcttcttcttgggagacccgccagagcccgctttggtgatgcggcc

SEQ ID NO: 86 - DNA, 48 bp, synthetic construct
  misc_feature 1..48: note = Description of Artificial Sequence: Synthetic oligonucleotide
  misc_feature 1..48: note = Category: primer
  misc_feature 1..48: note = Description: Amplify nMG4-2 (D28A) for pMGC expression plasmid
  source 1..48: mol_type = other DNA
acatccgaaagtgcgacacctgaatctgccatgcttcgtgagcccggt

SEQ ID NO: 87 - DNA, 53 bp, synthetic construct
  misc_feature 1..53: note = Description of Artificial Sequence: Synthetic oligonucleotide
  misc_feature 1..53: note = Category: primer
  misc_feature 1..53: note = Description: Amplify nMG4-2 (D28A) for pMGC expression plasmid
  source 1..53: mol_type = other DNA
atctgaaagattggtagaaccaccagacccatcgttagcataacgaatttccc

SEQ ID NO: 88 - DNA, 56 bp, synthetic construct
  misc_feature 1..56: note = Description of Artificial Sequence: Synthetic oligonucleotide
  misc_feature 1..56: note = Category: primer
  misc_feature 1..56: note = Description: Amplify nMG7-1 (D10A) for pMGC expression plasmid
  source 1..56: mol_type = other DNA
acatccgaaagtgcgacacctgaatctgccatgagtaataaaacaattttaggcct

SEQ ID NO: 89 - DNA, 51 bp, synthetic construct
  misc_feature 1..51: note = Description of Artificial Sequence: Synthetic oligonucleotide
  misc_feature 1..51: note = Category: primer
  misc_feature 1..51: note = Description: Amplify nMG7-1 (D10A) for pMGC expression plasmid
  source 1..51: mol_type = other DNA
atctgaaagattggtagaaccaccagaccctttgggcaagcctaaaacatc

SEQ ID NO: 90 - DNA, 54 bp, synthetic construct
  misc_feature 1..54: note = Description of Artificial Sequence: Synthetic oligonucleotide
  misc_feature 1..54: note = Category: primer
  misc_feature 1..54: note = Description: Amplify nMG16-1 (D9A) for pMGC expression plasmid
  source 1..54: mol_type = other DNA
acatccgaaagtgcgacacctgaatctgccatgattaagaatatcttgggtctg

SEQ ID NO: 91 - DNA, 48 bp, synthetic construct
  misc_feature 1..48: note = Description of Artificial Sequence: Synthetic oligonucleotide
  misc_feature 1..48: note = Category: primer
  misc_feature 1..48: note = Description: Amplify nMG16-1 (D9A) for pMGC expression plasmid
  source 1..48: mol_type = other DNA
atctgaaagattggtagaaccaccagaccccgctttggtgatgcggcc

SEQ ID NO: 92 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGA4-2_sgRNA spacer 1
  source 1..50: mol_type = other DNA
cacagcccgttttcatcatatttaatcactatagtgagtcgtattaaggt

SEQ ID NO: 93 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGA4-2_sgRNA spacer 1
  source 1..50: mol_type = other DNA
tcactatagtgattaaatatgatgaaaacgggctgtggtttgatgggttc

SEQ ID NO: 94 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGA4-2_sgRNA spacer 2
  source 1..50: mol_type = other DNA
cacagccttcacttacgccaatgtcgttctatagtgagtcgtattaaggt

SEQ ID NO: 95 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGA4-2_sgRNA spacer 2
  source 1..50: mol_type = other DNA
tcactatagaacgacattggcgtaagtgaaggctgtggtttgatgggttc

SEQ ID NO: 96 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGA4-2_sgRNA spacer 3
  source 1..50: mol_type = other DNA
cacagcggccataattcaattcgcgcgtctatagtgagtcgtattaaggt

SEQ ID NO: 97 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGA4-2_sgRNA spacer 3
  source 1..50: mol_type = other DNA
tcactatagacgcgcgaattgaattatggccgctgtggtttgatgggttc

SEQ ID NO: 98 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGA7-1_sgRNA spacer 1
  source 1..50: mol_type = other DNA
cacaactccgccacatatcctgatcttcctatagtgagtcgtattaaggt

SEQ ID NO: 99 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGA7-1_sgRNA spacer 1
  source 1..50: mol_type = other DNA
ctataggaagatcaggatatgtggcggagttgtgaatggctttcagaaat

SEQ ID NO: 100 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGA7-1_sgRNA spacer 2
  source 1..50: mol_type = other DNA
cacaacacagtttcgggttttcgacgttctatagtgagtcgtattaaggt

SEQ ID NO: 101 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGA7-1_sgRNA spacer 2
  source 1..50: mol_type = other DNA
tatagaacgtcgaaaacccgaaactgtgttgtgaatggctttcagaaatg

SEQ ID NO: 102 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGA7-1_sgRNA spacer 3
  source 1..50: mol_type = other DNA
cacaacattggcaccatgccgtgggtttctatagtgagtcgtattaaggt

SEQ ID NO: 103 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGA7-1_sgRNA spacer 3
  source 1..50: mol_type = other DNA
tatagaaacccacggcatggtgccaatgttgtgaatggctttcagaaatg

SEQ ID NO: 104 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGA16-1_sgRNA spacer 1
  source 1..50: mol_type = other DNA
cacaaccttcgctattacgccagctggcctatagtgagtcgtattaaggt

SEQ ID NO: 105 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGA16-1_sgRNA spacer 1
  source 1..50: mol_type = other DNA
tataggccagctggcgtaatagcgaaggttgtgtatggaaacatacacaa

SEQ ID NO: 106 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGA16-1_sgRNA spacer 2
  source 1..50: mol_type = other DNA
cacaacctggcgacctgcgtttcaccctctatagtgagtcgtattaaggt

SEQ ID NO: 107 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGA16-1_sgRNA spacer 2
  source 1..50: mol_type = other DNA
tatagagggtgaaacgcaggtcgccaggttgtgtatggaaacatacacaa

SEQ ID NO: 108 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGA16-1_sgRNA spacer 3
  source 1..50: mol_type = other DNA
cacaacggttttcgacgttcagacgtagctatagtgagtcgtattaaggt

SEQ ID NO: 109 - DNA, 49 bp, synthetic construct
  misc_feature 1..49: note = Description of Artificial Sequence: Synthetic oligonucleotide
  misc_feature 1..49: note = Category: primer
  misc_feature 1..49: note = Description: Amplify MGA16-1_sgRNA spacer 3
  source 1..49: mol_type = other DNA
atagctacgtctgaacgtcgaaaaccgttgtgtatggaaacatacacaa

SEQ ID NO: 110 - DNA, 41 bp, synthetic construct
  misc_feature 1..41: note = Description of Artificial Sequence: Synthetic oligonucleotide
  misc_feature 1..41: note = Category: primer
  misc_feature 1..41: note = Description: Amplify ABE8.17m_sgRNA spacer 1
  source 1..41: mol_type = other DNA
atccacatctgtgaaagaaactatagtgagtcgtattagga

SEQ ID NO: 111 - DNA, 45 bp, synthetic construct
  misc_feature 1..45: note = Description of Artificial Sequence: Synthetic oligonucleotide
  misc_feature 1..45: note = Category: primer
  misc_feature 1..45: note = Description: Amplify ABE8.17m_sgRNA spacer 1
  source 1..45: mol_type = other DNA
tttctttcacagatgtggatgttttagagctagaaatagcaagtt

SEQ ID NO: 112 - DNA, 41 bp, synthetic construct
  misc_feature 1..41: note = Description of Artificial Sequence: Synthetic oligonucleotide
  misc_feature 1..41: note = Category: primer
  misc_feature 1..41: note = Description: Amplify ABE8.17m_sgRNA spacer 2
  source 1..41: mol_type = other DNA
ctcatccgccacatatcctgctatagtgagtcgtattagga

SEQ ID NO: 113 - DNA, 45 bp, synthetic construct
  misc_feature 1..45: note = Description of Artificial Sequence: Synthetic oligonucleotide
  misc_feature 1..45: note = Category: primer
  misc_feature 1..45: note = Description: Amplify ABE8.17m_sgRNA spacer 2
  source 1..45: mol_type = other DNA
caggatatgtggcggatgaggttttagagctagaaatagcaagtt

SEQ ID NO: 114 - DNA, 41 bp, synthetic construct
  misc_feature 1..41: note = Description of Artificial Sequence: Synthetic oligonucleotide
  misc_feature 1..41: note = Category: primer
  misc_feature 1..41: note = Description: Amplify ABE8.17m_sgRNA spacer 3
  source 1..41: mol_type = other DNA
atcgcgtgggcgtattcgcactatagtgagtcgtattagga

SEQ ID NO: 115 - DNA, 45 bp, synthetic construct
  misc_feature 1..45: note = Description of Artificial Sequence: Synthetic oligonucleotide
  misc_feature 1..45: note = Category: primer
  misc_feature 1..45: note = Description: Amplify ABE8.17m_sgRNA spacer 3
  source 1..45: mol_type = other DNA
tgcgaatacgcccacgcgatgttttagagctagaaatagcaagtt

SEQ ID NO: 116 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGC4-2_spacer 1
  source 1..50: mol_type = other DNA
cacagcgataatgcgaacagcgcacggcctatagtgagtcgtattaaggt

SEQ ID NO: 117 - DNA, 49 bp, synthetic construct
  misc_feature 1..49: note = Description of Artificial Sequence: Synthetic oligonucleotide
  misc_feature 1..49: note = Category: primer
  misc_feature 1..49: note = Description: Amplify MGC4-2_spacer 1
  source 1..49: mol_type = other DNA
cactataggccgtgcgctgttcgcattatcgctgtggtttgatgggttc

SEQ ID NO: 118 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGC4-2_spacer 2
  source 1..50: mol_type = other DNA
cacagcctgacgaaacgcctgccagtatctatagtgagtcgtattaaggt

SEQ ID NO: 119 - DNA, 49 bp, synthetic construct
  misc_feature 1..49: note = Description of Artificial Sequence: Synthetic oligonucleotide
  misc_feature 1..49: note = Category: primer
  misc_feature 1..49: note = Description: Amplify MGC4-2_spacer 2
  source 1..49: mol_type = other DNA
cactatagatactggcaggcgtttcgtcaggctgtggtttgatgggttc

SEQ ID NO: 120 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGC4-2_spacer 3
  source 1..50: mol_type = other DNA
cacagcgcgtgggacgcggcggggagtgctatagtgagtcgtattaaggt

SEQ ID NO: 121 - DNA, 49 bp, synthetic construct
  misc_feature 1..49: note = Description of Artificial Sequence: Synthetic oligonucleotide
  misc_feature 1..49: note = Category: primer
  misc_feature 1..49: note = Description: Amplify MGC4-2_spacer 3
  source 1..49: mol_type = other DNA
cactatagcactccccgccgcgtcccacgcgctgtggtttgatgggttc

SEQ ID NO: 122 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGC7-1_spacer 1
  source 1..50: mol_type = other DNA
cacaacgacgtagtgtgacgcgatcggcctatagtgagtcgtattaaggt

SEQ ID NO: 123 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGC7-1_spacer 1
  source 1..50: mol_type = other DNA
tataggccgatcgcgtcacactacgtcgttgtgaatggctttcagaaatg

SEQ ID NO: 124 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGC7-1_spacer 2
  source 1..50: mol_type = other DNA
cacaactcgccggtagccagcgcggatcctatagtgagtcgtattaaggt

SEQ ID NO: 125 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGC7-1_spacer 2
  source 1..50: mol_type = other DNA
tataggatccgcgctggctaccggcgagttgtgaatggctttcagaaatg

SEQ ID NO: 126 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGC7-1_spacer 3
  source 1..50: mol_type = other DNA
cacaaccatactgcaccgggcgggaaggctatagtgagtcgtattaaggt

SEQ ID NO: 127 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGC7-1_spacer 3
  source 1..50: mol_type = other DNA
tatagccttcccgcccggtgcagtatggttgtgaatggctttcagaaatg

SEQ ID NO: 128 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGC16-1_spacer 1
  source 1..50: mol_type = other DNA
cacaacaccgtaatgggataggtcacgtctatagtgagtcgtattaaggt

SEQ ID NO: 129 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGC16-1_spacer 1
  source 1..50: mol_type = other DNA
tatagacgtgacctatcccattacggtgttgtgtatggaaacatacacaa

SEQ ID NO: 130 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGC16-1_spacer 2
  source 1..50: mol_type = other DNA
cacaactccgtgggaacaaacggcggatctatagtgagtcgtattaaggt

SEQ ID NO: 131 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGC16-1_spacer 2
  source 1..50: mol_type = other DNA
atagatccgccgtttgttcccacggagttgtgtatggaaacatacacaat

SEQ ID NO: 132 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGC16-1_spacer 3
  source 1..50: mol_type = other DNA
cacaactgcttcaatcagcgtgccgtcgctatagtgagtcgtattaaggt

SEQ ID NO: 133 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MGC16-1_spacer 3
  source 1..50: mol_type = other DNA
atagcgacggcacgctgattgaagcagttgtgtatggaaacatacacaat

SEQ ID NO: 134 - DNA, 41 bp, synthetic construct
  misc_feature 1..41: note = Description of Artificial Sequence: Synthetic oligonucleotide
  misc_feature 1..41: note = Category: primer
  misc_feature 1..41: note = Description: Amplify BE3_sgRNA spacer 1
  source 1..41: mol_type = other DNA
tgtggagcgacatccagaggctatagtgagtcgtattagga

SEQ ID NO: 135 - DNA, 45 bp, synthetic construct
  misc_feature 1..45: note = Description of Artificial Sequence: Synthetic oligonucleotide
  misc_feature 1..45: note = Category: primer
  misc_feature 1..45: note = Description: Amplify BE3_sgRNA spacer 1
  source 1..45: mol_type = other DNA
cctctggatgtcgctccacagttttagagctagaaatagcaagtt

SEQ ID NO: 136 - DNA, 41 bp, synthetic construct
  misc_feature 1..41: note = Description of Artificial Sequence: Synthetic oligonucleotide
  misc_feature 1..41: note = Category: primer
  misc_feature 1..41: note = Description: Amplify BE3_sgRNA spacer 2
  source 1..41: mol_type = other DNA
taattcaattcgcgcgtcccctatagtgagtcgtattagga

SEQ ID NO: 137 - DNA, 45 bp, synthetic construct
  misc_feature 1..45: note = Description of Artificial Sequence: Synthetic oligonucleotide
  misc_feature 1..45: note = Category: primer
  misc_feature 1..45: note = Description: Amplify BE3_sgRNA spacer 2
  source 1..45: mol_type = other DNA
gggacgcgcgaattgaattagttttagagctagaaatagcaagtt

SEQ ID NO: 138 - DNA, 41 bp, synthetic construct
  misc_feature 1..41: note = Description of Artificial Sequence: Synthetic oligonucleotide
  misc_feature 1..41: note = Category: primer
  misc_feature 1..41: note = Description: Amplify BE3_sgRNA spacer 3
  source 1..41: mol_type = other DNA
agtcacgacgttgtaaaacgctatagtgagtcgtattagga

SEQ ID NO: 139 - DNA, 45 bp, synthetic construct
  misc_feature 1..45: note = Description of Artificial Sequence: Synthetic oligonucleotide
  misc_feature 1..45: note = Category: primer
  misc_feature 1..45: note = Description: Amplify BE3_sgRNA spacer 3
  source 1..45: mol_type = other DNA
cgttttacaacgtcgtgactgttttagagctagaaatagcaagtt

SEQ ID NO: 140 - DNA, 25 bp, synthetic construct
  misc_feature 1..25: note = Description of Artificial Sequence: Synthetic oligonucleotide
  misc_feature 1..25: note = Category: primer
  misc_feature 1..25: note = Description: For lacZ sequencing
  source 1..25: mol_type = other DNA
cgaacatccaaaagtttgtgttttt

SEQ ID NO: 141 - DNA, 19 bp, synthetic construct
  misc_feature 1..19: note = Description of Artificial Sequence: Synthetic oligonucleotide
  misc_feature 1..19: note = Category: primer
  misc_feature 1..19: note = Description: For lacZ sequencing
  source 1..19: mol_type = other DNA
tgagcgcatttttacgcgc

SEQ ID NO: 142 - DNA, 18 bp, synthetic construct
  misc_feature 1..18: note = Description of Artificial Sequence: Synthetic oligonucleotide
  misc_feature 1..18: note = Category: primer
  misc_feature 1..18: note = Description: For lacZ sequencing
  source 1..18: mol_type = other DNA
gaaaacggcaacccgtgg

SEQ ID NO: 143 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify sgRNA expression cassette
  source 1..50: mol_type = other DNA
ccgccgccgcaaggaatggtttaattaattacggccagtcatgcataatc

SEQ ID NO: 144 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify sgRNA expression cassette
  source 1..50: mol_type = other DNA
gactgttgggcgccatctccttgcatgctcactgagcctccacctagcct

SEQ ID NO: 145-147 - skipped (3 entries)

SEQ ID NO: 148 - protein, 7 aa, synthetic construct
  REGION 1..7: note = Description of Artificial Sequence: Synthetic peptide
  REGION 1..7: note = Category: NLS
  REGION 1..7: note = Description: SV40
PKKKRKV

SEQ ID NO: 149 - protein, 16 aa, unidentified
  REGION 1..16: note = Description of Unknown: nucleoplasmin bipartite NLS sequence
  REGION 1..16: note = Category: NLS
  REGION 1..16: note = Description: nucleoplasmin bipartite NLS
KRPAATKKAGQAKKKK

SEQ ID NO: 150 - protein, 9 aa, unidentified
  REGION 1..9: note = Description of Unknown: c-myc NLS sequence
  REGION 1..9: note = Category: NLS
  REGION 1..9: note = Description: c-myc NLS
PAAKRVKLD

SEQ ID NO: 151 - protein, 11 aa, unidentified
  REGION 1..11: note = Description of Unknown: c-myc NLS sequence
  REGION 1..11: note = Category: NLS
  REGION 1..11: note = Description: c-myc NLS
RQRRNELKRSP

SEQ ID NO: 152 - protein, 38 aa, Homo sapiens
  REGION 1..38: note = Category: NLS
  REGION 1..38: note = Description: hRNPA1 M9 NLS
NQSSNFGPMKGGNFGGRSSGPYGGGGQYFAKPRNQGGY

SEQ ID NO: 153 - protein, 42 aa, unidentified
  REGION 1..42: note = Description of Unknown: Importin-alpha IBB domain sequence
  REGION 1..42: note = Category: NLS
  REGION 1..42: note = Description: Importin-alpha IBB domain
RMRIZFKNKGKDTAELRRRRVEVSVELRKAKKDEQILKRRNV

SEQ ID NO: 154 - protein, 8 aa, unidentified
  REGION 1..8: note = Description of Unknown: Myoma T protein sequence
  REGION 1..8: note = Category: NLS
  REGION 1..8: note = Description: Myoma T protein
VSRKRPRP

SEQ ID NO: 155 - protein, 8 aa, unidentified
  REGION 1..8: note = Description of Unknown: Myoma T protein sequence
  REGION 1..8: note = Category: NLS
  REGION 1..8: note = Description: Myoma T protein
PPKKARED

SEQ ID NO: 156 - protein, 8 aa, unidentified
  REGION 1..8: note = Description of Unknown: p53 sequence
  REGION 1..8: note = Category: NLS
  REGION 1..8: note = Description: p53
PQPKKKPL

SEQ ID NO: 157 - protein, 12 aa, Mus sp.
  REGION 1..12: note = Category: NLS
  REGION 1..12: note = Description: mouse c-abl IV
SALIKKKKKMAP

SEQ ID NO: 158 - protein, 5 aa, Influenza virus
  REGION 1..5: note = Category: NLS
  REGION 1..5: note = Description: influenza virus NS1
DRLRR

SEQ ID NO: 159 - protein, 7 aa, Influenza virus
  REGION 1..7: note = Category: NLS
  REGION 1..7: note = Description: influenza virus NS1
PKQKKRK

SEQ ID NO: 160 - protein, 10 aa, Hepatitis delta virus
  REGION 1..10: note = Category: NLS
  REGION 1..10: note = Description: Hepatitis virus delta antigen
RKLKKKIKKL

SEQ ID NO: 161 - protein, 10 aa, Mus sp.
  REGION 1..10: note = Category: NLS
  REGION 1..10: note = Description: mouse Mx1 protein
REKKKFLKRR

SEQ ID NO: 162 - protein, 20 aa, Homo sapiens
  REGION 1..20: note = Category: NLS
  REGION 1..20: note = Description: human poly(ADP-ribose) polymerase
KRKGDEVDGVDEVAKKKSKK

SEQ ID NO: 163 - protein, 17 aa, Homo sapiens
  REGION 1..17: note = Category: NLS
  REGION 1..17: note = Description: steroid hormone receptor (human) glucocorticoid
RKCLQAGMNLEARKTKK